(19) United States
(12) Patent Application Publication
(10) Pub. No.: US 2005/0282812 A1
(43) Pub. Date: Dec. 22, 2005

(54) Inhibitors of cholesteryl ester transfer protein

(76) Inventors: Zachary Jones, Broomfield, CO (US); Robert Groneberg, Boulder, CO (US); Mark Drew, Tucson, AZ (US); Charles Todd Eary, Longmont, CO (US)

Correspondence Address: HOGAN & HARTSON LLP, ONE TABOR CENTER, SUITE 1500, 1200 SEVENTEENTH ST, DENVER, CO, 80202, US

(21) Appl. No.: 11/155,001

(22) Filed: Jun. 16, 2005

Related U.S. Application Data
(60) Provisional application No. 60/581,049, filed on Jun. 18, 2004.

(51) Int. Cl. A61K 31/498; C07D 241/36

(52) U.S. Cl. 514/249; 544/353

(57) ABSTRACT

This invention relates to inhibitors of CETP and methods for producing these inhibitors. The invention also provides pharmaceutical compositions comprising the inhibitors of the invention and methods of utilizing the inhibitors and pharmaceutical compositions in the treatment and prevention of various disorders mediated by CETP.

### CROSS-REFERENCE TO RELATED APPLICATIONS

This application claims priority to U.S. Provisional Application No. 60/581,049, filed Jun. 18, 2004, which is incorporated herein by reference in its entirety.

### BACKGROUND OF THE INVENTION

1. Field of the Invention

This invention relates to cholesteryl ester transfer protein (CETP) inhibitors, pharmaceutical compositions containing such inhibitors, and the use of such inhibitors to treat certain disease/conditions optionally in combination with certain therapeutic agents.

2. Description of the State of the Art

Atherosclerosis and its associated coronary artery disease (CAD) is the leading cause of mortality in the industrialized world. Despite attempts to modify secondary risk factors (smoking, obesity, lack of exercise) and treatment of dyslipidemia with dietary modification and drug therapy, coronary heart disease (CHD) remains the most common cause of death in the U.S., where cardiovascular disease accounts for 44% of all deaths, with 53% of these associated with atherosclerotic coronary heart disease.

The risk for development of this condition has been shown to be strongly correlated with certain plasma lipid levels. While elevated LDL cholesterol may be the most recognized form of dyslipidemia, it is by no means the only significant lipid associated contributor to CHD. Low HDL cholesterol is also a known risk factor for CHD (Gordon, D. J., et al., Circulation (1989) 79: 8-15).

Therapies to raise HDL cholesterol levels have been limited. HMG-CoA reductase inhibitors and fibrates only raise HDL cholesterol levels slightly and while niacin can more significantly raise HDL cholesterol levels, side effects severely reduce its tolerability and compliance. Therefore, alternative therapies to raise HDL cholesterol are needed.

Among the many factors controlling plasma levels of these disease-dependent principles, cholesteryl ester transfer protein (CETP) activity affects all three. Cholesteryl ester transfer protein (CETP) is a 70,000 dalton glycoprotein present in the plasma of humans and other animal species. The role of CETP role is to transfer cholesterol ester, triglyceride and to a limited extent phospholipids between plasma lipoprotein particles. The lipoprotein particles involved include high density lipoprotein (HDL), low density lipoprotein (LDL), very low density lipoprotein (VLDL) and chylomicrons. This effect on lipoprotein profile is believed to be proatherogenic, especially in subjects whose lipid profile constitutes an increased risk for CHD. Since CETP is involved in the homeostasis of the plasma lipoprotein pool, its regulation by inhibition in the plasma compartment should allow for an altering of the circulating levels of these lipoproteins.

Clinical trials utilizing inhibitors of CETP have demonstrated the ability to raise circulating HDL cholesterol levels by this mechanism. One study employing a CETP inhibitor demonstrated a 34% increase in HDL cholesterol after 4 weeks using a 900 mg/day dose (Circulation, (2002) 105:2159). Evaluation of another CETP inhibitor showed that after four weeks at the highest dose, a 106% elevation in HDL cholesterol using a 120 mg dose twice daily (N. Engl. J. Med., (2004) 350:1505-1515). Elevating plasma HDL cholesterol levels by inhibiting the activity of CETP may provide an anti-atherogenic benefit in humans. Although this has not yet been proven in humans, in rabbits, a CETP inhibitor was shown to be anti-athereogenic (Nature, (2000) 406: 203-207).

### SUMMARY OF THE INVENTION

This invention provides cholesteryl ester transfer protein (CETP) inhibitors, methods to produce these compounds, and pharmaceutical compositions containing them for treating a CETP-mediated disorder or condition. The disorder or condition includes, but is not limited to, cerebrovascular disease, coronary artery disease, hypertension, ventricular dysfunction, cardiac arrhythmia, pulmonary vascular disease, peripheral vascular disease, reno-vascular disease, renal disease, splanchnic vascular disease, vascular hemostatic disease, diabetes, inflammatory disease, autoimmune disorders and other systemic disease indications, immune function modulation, pulmonary disease, anti-oxidant disease, sexual dysfunction, cognitive dysfunction, schistosomiasis and cancer in a mammal. CETP inhibitors of the invention may be useful for the treatment of atherosclerosis, peripheral vascular disease and dyslipidemias, including hyperbetalipoproteinemia, hypoalphalipoproteinemia, hypercholesterolemia, familial hypercholesterolemia and hypertriglyceridemia.

In general, the invention relates to CETP inhibitors of the general Formula I: 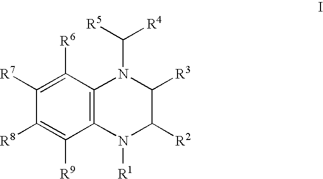
and metabolites, solvates, tautomers, resolved enantiomers, diastereomers, pharmaceutically acceptable salts and pharmaceutically acceptable prodrugs thereof, wherein:
R¹ is Zₙ-(C═O)OR¹⁰, Zₙ-(C═O)R¹⁰, Zₙ(C═O)Zₙ(C═O)OR¹⁰, Zₙ-NR¹⁰R¹¹, Zₙ-(C═O)NR¹⁰R¹¹, Zₙ-SOR¹⁰, Zₙ-SO₂R¹⁰, alkyl, allyl, alkenyl, alkynyl, heteroalkyl, heteroallyl, heteroalkenyl, heteroalkynyl, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, Zₙ-cycloalkyl, Zₙ-heterocycloalkyl and Zₙ-Ar are optionally substituted with one or more groups independently selected from F, Zₙ-COOR¹⁰, ZₙOR¹⁰, Zₙ-NR¹⁰R¹¹, Zₙ-(C═O)NR¹⁰R¹¹, oxo and alkyl;
R² and R³ are independently H, OH, F, Cl, Br, I, CF₃, Zₙ-NR¹⁰R¹¹, Zₙ-NR¹⁰(C═O)R¹¹, Zₙ-SO₂R¹⁰, Zₙ-SOR¹⁰, Zₘ-SR¹⁰, Zₙ-OR¹⁰, Zₙ-(C═O)R¹⁰, Zₙ-(C═O)OR¹⁰, Zₙ-O(C═O)R¹⁰, alkyl, allyl, alkenyl, alkynyl, heteroalkyl, heteroallyl, heteroalkenyl, heteroalkynyl, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar are optionally substituted with one or more groups independently selected from OR¹⁰ and SR¹⁰;
or R¹ and R² together with the atoms to which they are attached form a substituted or unsubstituted, saturated or partially unsaturated 5 or 6-membered heterocyclic ring;
R⁴ is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, OR′, SR′F, Cl, Br, I, CN and NO₂, wherein said alkyl is optionally substituted with one or more groups independently selected from C(═O)OR′, CN, NR′R″, C(═O)NR′R″, cycloalkyl, OH, F and alkyl;
R⁵ is heteroaryl optionally substituted with one or more groups independently selected from H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₙ-NR′R″, Zₙ-C(═O)NR′R″, Zₙ-OR′, F, Cl, Br or I;
R⁶ and R⁷, R⁷ and R⁸ and R⁹ are independently H, OH, F, Cl, Br, I, CF₃, OCF₃, OCF₂H, Zn-NR¹⁰R¹¹, Zₙ-(C═O)NR¹⁰R¹¹, Zₙ-SO₂R¹⁰, Zₙ-SOR¹⁰, Zₙ-SR¹⁰, Zₙ-OR¹⁰, Zₙ-(C═O)R¹⁰, Zₙ-(C═O)OR¹⁰, Zₙ-O—(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar,
or R⁶ and R⁷, R⁷ and R⁸, and/or R⁸ and R⁹ together with the atoms to which they are attached form a carbocyclic or heterocyclic ring, wherein said carbocyclic and heterocyclic rings are optionally substituted with one or more groups independently selected from alkyl and F;
R¹⁰ and R¹¹ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, cycloalkyl, heterocycloalkyl and Ar are optionally substituted with one or more groups independently selected from alkyl, OR′ and Ar,
or R¹⁰ and R¹¹ together with the atoms to which they are attached form a substituted or unsubstituted, saturated or partially unsaturated 5 or 6-membered heterocyclic ring;
Z is alkylene having from 1 to 4 carbons, or alkenylene or alkynylene each having from 2 to 4 carbons, wherein said alkylene, alkenylene and alkynylene are optionally substituted;
Ar is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, OR′, SR′F, Cl, Br, I, CN and NO₂, wherein said alkyl is optionally substituted with one or more groups independently selected from C(═O)OR′, CN, NR′R″, C(═O)NR′R″, cycloalkyl, OR′, F and alkyl;
R′ and R″ are independently H or C₁-C₁₀ alkyl, wherein said alkyl is optionally substituted with one or more F;
m is 1 or 2; and
n is 0, 1, or 2.

This invention further provides compounds having Formula II 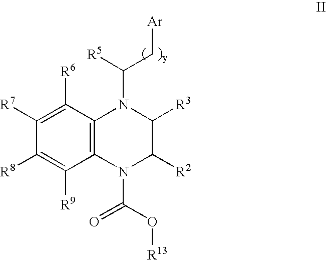
and metabolites, solvates, tautomers, resolved enantiomers, diastereomers, pharmaceutically acceptable salts and pharmaceutically acceptable prodrugs thereof, wherein:
R² is C₁-C₄ alkyl, C₂-C₄ allyl, C₂-C₄ alkenyl, C₂-C₄ alkynyl, C₁-C₄ heteroalkyl, C₂-C₄ heteroalkenyl, or C₂-C₄ heteroalkynyl, wherein said alkyl, alkenyl, alkynyl, heteroalkyl, heteroalkenyl and heteroalkynyl are optionally substituted with one or more groups independently selected from OR¹⁰ and SR¹⁰;
R³ is H, CF₃, Zₙ-NR¹¹R¹², Zₙ-NR¹⁰(C═O)R¹⁰R¹¹, Zₙ-SO₂R¹⁰, Zₙ-SOR¹⁰, Zₙ-SR¹⁰, Zₙ-OR¹⁰, Zₙ-(C═O)R¹⁰, Zₙ-(C═O)OR¹⁰, Zₙ-O(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar are optionally substituted with one or more groups independently selected from OR¹⁰ and SR¹⁰;
R⁵ is 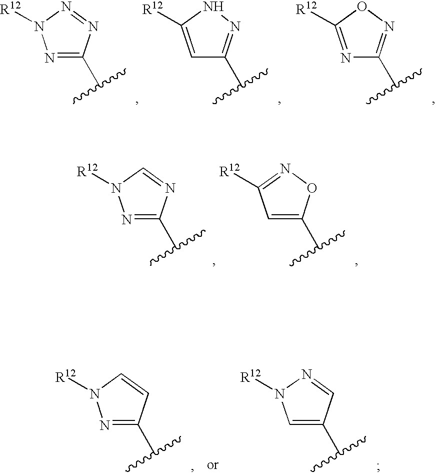
R⁶, R⁷, R⁸ and R⁹ are independently H, OH, F, Cl, Br, I, CF₃, OCF₃, OCF₂H, Zn-NR¹⁰R¹¹, Zₙ-(C═O)NR¹⁰R¹¹, Zₙ-SO₂R¹⁰, Zₙ-SO₂R¹⁰, Zₙ—SR¹⁰, Zₙ-OR¹⁰, Zₙ-(C═O)R¹⁰, Zₙ-(C═O)OR¹⁰, Zₙ-O—(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar,
or R⁶ and R⁷, R⁷ and R⁸, and/or R⁸ and R⁹ together with the atoms to which they are attached form a carbocyclic or heterocyclic ring, wherein said carbocyclic and heterocyclic rings are optionally substituted with one or more groups independently selected from alkyl and F;
R¹⁰ and R¹¹ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, cycloalkyl, heterocycloalkyl and Ar are optionally substituted with one or more groups independently selected from alkyl, OR′ and Ar,
or R¹⁰ and R¹¹ together with the atoms to which they are attached form a substituted or unsubstituted, saturated or partially unsaturated 5 or 6-membered heterocyclic ring;
R¹² is H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₘ-NR′R″, Zₙ-C(═O)NR′R″ or Zₘ-OR′;
R¹³ is C₁-C₄ alkyl, C₂-C₄ allyl, C₂-C₄ alkenyl, C₂-C₄ alkynyl, Zₙ-cycloalkyl, or Zₙ-Ar, wherein said alkyl, allyl, alkenyl, alkynyl, cycloalkyl and Ar are optionally substituted with one or more groups independently selected from OR′ and alkyl;
Ar is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, —OR′, SR′F, Cl, Br, I, CN and NO₂, wherein said alkyl is optionally substituted with one or more groups independently selected from C(═O)OR′, CN, NR′R″, C(═O)NR′R″, cycloalkyl, OR′, F and alkyl;
R′ and R″ are independently H or C₁-C₁₀ alkyl, wherein said alkyl is optionally substituted with one or more F;
Z is alkylene having from 1 to 4 carbons, or alkenylene or alkynylene each having from 2 to 4 carbons, wherein said alkylene, alkenylene and alkynylene are optionally substituted;
m is 1 or 2;
n is 0, 1, or 2; and
y is 0 or 1.

This invention further provides compounds having Formula III 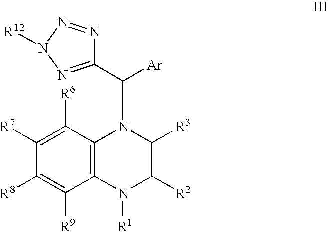
and metabolites, solvates, tautomers, resolved enantiomers, diastereomers, pharmaceutically acceptable salts and pharmaceutically acceptable prodrugs thereof, wherein:
R¹ is Zₙ-(C═O)OR¹⁰, Zₙ-(C═O)R¹⁰, Zₙ(C═O)Zₙ(C═O)OR¹⁰, Zₙ—NR¹⁰R¹¹, Zₙ—(C═O)NR¹⁰R¹¹, Zₙ-SOR¹⁰, Zₙ—SO₂R¹⁰, alkyl, allyl, alkenyl, alkynyl, heteroalkyl, heteroallyl, heteroalkenyl, heteroalkynyl, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, Zₙ-cycloalkyl, Zₙ-heterocycloalkyl and Zₙ-Ar are optionally substituted with one or more groups independently selected from F, Zₙ-COOR¹⁰, ZNOR¹⁰, Zₙ-NR¹⁰R¹¹, Zₙ-(C═O)NR¹⁰R¹¹, oxo and alkyl;
R² and R³ are independently H, OH, F, Cl, Br, I, CF₃, Zₙ-NR¹⁰R¹¹, Zₙ-NR¹⁰(C═O)R¹¹, Zₙ-SO₂R¹⁰, Zₙ-SOR¹⁰, Zₘ-SR¹⁰, Zₙ-OR¹⁰, Zₙ-(C═O)R¹⁰, Zₙ-(C═O)OR¹⁰, Zₙ-O(C═O)R¹⁰, alkyl, allyl, alkenyl, alkynyl, heteroalkyl, heteroallyl, heteroalkenyl, heteroalkynyl, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar are optionally substituted with one or more groups independently selected from OR¹⁰ and SR¹⁰,
or R¹ and R² together with the atoms to which they are attached form a substituted or unsubstituted, saturated or partially unsaturated 5 or 6-membered heterocyclic ring;
R⁶, R⁷, R⁸ and R⁹ are independently H, OH, F, Cl, Br, I, CF₃, OCF₃, OCF₂H, Zn-NR¹⁰R¹¹, Zₙ-(C═O)NR¹⁰R¹¹, Zₙ-SO₂R¹⁰, Zₙ-SOR¹⁰, Zₙ-SR¹⁰, Zₙ-OR¹⁰, Zₙ-(C═O)R¹⁰, Zₙ-(C═O)OR¹⁰, Zₙ-O—(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar,
or R⁶ and R⁷, R⁷ and R⁸, and/or R⁸ and R⁹ together with the atoms to which they are attached form a carbocyclic or heterocyclic ring, wherein said carbocyclic and heterocyclic rings are optionally substituted with one or more groups independently selected from alkyl and F;
R¹⁰ and R¹¹ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, cycloalkyl, heterocycloalkyl and Ar are optionally substituted with one or more groups independently selected from alkyl, OR′ and Ar,
or R¹⁰ and R¹¹ together with the atoms to which they are attached form a substituted or unsubstituted, saturated or partially unsaturated 5 or 6-membered heterocyclic ring;
R¹² is H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₘ-NR′R″, Zₙ-C(═O)NR′R″ or Zₘ-OR′;
Z is alkylene having from 1 to 4 carbons, or alkenylene or alkynylene each having from 2 to 4 carbons, wherein said alkylene, alkenylene and alkynylene are optionally substituted;
Ar is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, —OR′, SR′F, Cl, Br, I, CN and NO₂, wherein said alkyl is optionally substituted with one or more groups independently selected from C(═O)OR′, CN, —NR′R″, C(═O)NR′R″, cycloalkyl, OR′, F and alkyl;
R′ and R″ are independently H or C₁-C₁₀ alkyl, wherein said alkyl is optionally substituted with one or more F;
m is 1 or 2; and
n is 0, 1, or 2.

In a further aspect the present invention provides a method of providing a CETP inhibitory effect comprising administering to a warm-blooded animal an effective amount of a compound of Formula I, II or III or a pharmaceutically acceptable salt or in vivo cleavable prodrug thereof, or a pharmaceutical composition comprising said compound.

In a further aspect the present invention provides methods of treating or preventing a CETP-mediated condition, comprising administering to a human or animal in need thereof a compound of Formula I, II or III or a pharmaceutically-acceptable salt or in vivo cleavable prodrug thereof, or a pharmaceutical composition comprising said compound, in an amount effective to treat or prevent said CETP-mediated condition.

The inventive compounds may be used advantageously in combination with other known therapeutic agents.

The invention also relates to pharmaceutical compositions comprising an effective amount of an agent selected from a compound of Formulas I, II or III or a pharmaceutically acceptable prodrug, pharmaceutically active metabolite, or pharmaceutically acceptable salt thereof.

This invention further provides kits comprising a compound of Formula I, II or III or a pharmaceutically acceptable salt or in vivo cleavable prodrug thereof, or a pharmaceutical composition comprising said compound.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### BRIEF DESCRIPTION OF THE FIGURES

The accompanying drawings, which are incorporated herein and form a part of the specification, illustrate non-limiting embodiments of the present invention, and together with the description, serve to explain the principles of the invention.

In the Figures:

FIG. 1 shows a reaction scheme for the synthesis of compound 5.

FIG. 2 shows a reaction scheme for the synthesis of compound 10.

FIG. 3 shows a reaction scheme for the synthesis of compounds 12, 15 and 16.

FIG. 4 shows a reaction scheme for the synthesis of compounds 22 and 23.

FIG. 5 shows a reaction scheme for the synthesis of compounds 26 and 27.

FIG. 6 shows a reaction scheme for the synthesis of compound 30.

FIG. 7 shows a reaction scheme for the synthesis of compound 35.

FIG. 8 shows a reaction scheme for the synthesis of compounds 37-D 1, 37-D2, 38-D1 and 38-D2.

FIG. 9 shows a reaction scheme for the synthesis of compounds 39-D1, 39-D2, 139-D1 and 139-D2.

FIG. 10 shows a reaction scheme for the synthesis of compound 41.

FIG. 11 shows a reaction scheme for the synthesis of compound 53.

FIG. 12 shows a reaction scheme for the synthesis of compound 58.

FIG. 13 shows a reaction scheme for the synthesis of compound 61.

FIG. 14 shows a reaction scheme for the synthesis of compound 64.

FIG. 15 shows a reaction scheme for the synthesis of compound 68.

### DETAILED DESCRIPTION OF THE INVENTION

The inventive compounds of the present invention are useful for inhibiting CETP mediated events as described herein. In one embodiment, the method of treatment according to this invention results in a decrease in plasma small dense LDL, oxidized LDL, VLDL, apo(a) or Lp(a) or an increase in pre-beta HDL, HDL-1,-2 and 3 particles.

In general, the invention relates to CETP inhibitors of the general Formula I: 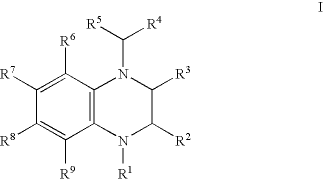
and metabolites, solvates, tautomers, resolved enantiomers, diastereomers, pharmaceutically acceptable salts and pharmaceutically acceptable prodrugs thereof, wherein:
R¹ is Zₙ-(C═O)OR¹⁰, Zₙ-(C═O)R¹⁰, Zₙ(C═O)Zₙ(C═O)OR¹⁰, Zₙ-NR¹⁰R¹¹, Zₙ-(C═O)NR¹⁰R¹¹, ZₙSOR¹⁰, Zₙ-SO₂R¹⁰, alkyl, allyl, alkenyl, alkynyl, heteroalkyl, heteroallyl, heteroalkenyl, heteroalkynyl, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, Zₙ-cycloalkyl, Zₙ-heterocycloalkyl and Zₙ-Ar are optionally substituted with one or more groups independently selected from F, Zₙ-COOR¹⁰, ZₙOR¹⁰, Zₙ-NR¹⁰R¹¹, Zₙ-(C═O)NR¹⁰R¹¹, oxo and alkyl;
R² and R³ are independently H, OH, F, Cl, Br, I, CF₃, Zₙ-NR¹⁰R¹¹, Zₙ-NR¹⁰(C═O)R¹¹, Zₙ-SO₂R¹⁰, Zₙ-SOR¹¹, Zₘ-SR¹⁰, Zₙ-OR¹⁰, Zₙ-(C═O)R¹⁰, Zₙ-(C═O)OR¹⁰, Zₙ-O(C═O)R¹⁰, alkyl, allyl, alkenyl, alkynyl, heteroalkyl, heteroallyl, heteroalkenyl, heteroalkynyl, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar are optionally substituted with one or more groups independently selected from OR¹⁰ and SR¹⁰;
or R¹ and R² together with the atoms to which they are attached form a substituted or unsubstituted, saturated or partially unsaturated 5 or 6-membered heterocyclic ring;
R⁴ is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, OR′, SR′F, Cl, Br, I, CN and NO₂, wherein said alkyl is optionally substituted with one or more groups independently selected from C(═O)OR′, CN, NR′R″, C(═O)NR′R″, cycloalkyl, OH, F and alkyl;
R⁵ is heteroaryl optionally substituted with one or more groups independently selected from H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₙ-NR′R″, Zₙ-C(═O)NR′R″, Zₙ-OR′, F, Cl, Br or I;
R⁶ and R⁷, R⁷ and R⁸ and R⁹ are independently H, OH, F, Cl, Br, I, CF₃, OCF₃, OCF₂H, Zₙ-NR¹⁰R¹¹, Zₙ-(C═O)NR¹⁰R¹¹, Zₙ-SO₂R¹⁰, Zₙ-SOR¹⁰, Zₙ-SR¹⁰, Zₙ-OR¹⁰, Zₙ-(C═O)R¹⁰, Zₙ-(C═O)OR¹⁰, Zₙ-O—(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar,
or R⁶ and R⁷, R⁷ and R⁸, and/or R⁸ and R⁹ together with the atoms to which they are attached form a carbocyclic or heterocyclic ring, wherein said carbocyclic and heterocyclic rings are optionally substituted with one or more groups independently selected from alkyl and F;
R¹⁰ and R¹¹ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, cycloalkyl, heterocycloalkyl and Ar are optionally substituted with one or more groups independently selected from alkyl, OR′ and Ar,
or R¹⁰ and R¹¹ together with the atoms to which they are attached form a substituted or unsubstituted, saturated or partially unsaturated 5 or 6-membered heterocyclic ring;
Z is alkylene having from 1 to 4 carbons, or alkenylene or alkynylene each having from 2 to 4 carbons, wherein said alkylene, alkenylene and alkynylene are optionally substituted;
Ar is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, OR′, SR′F, Cl, Br, I, CN and NO₂, wherein said alkyl is optionally substituted with one or more groups independently selected from C(═O)OR′, CN, NR′R″, C(═O)NR′R″, cycloalkyl, OR′, F and alkyl;
R′ and R″ are independently H or C₁-C₁₀ alkyl, wherein said alkyl is optionally substituted with one or more F;
m is 1 or 2; and
n is 0, 1, or 2.

In one embodiment, R¹ is Zₙ-(C═O)OR¹⁰, Zₙ-cycloalkyl, Zₙ-(C═O)OCH₂Ar, Zₙ-OR¹⁰,

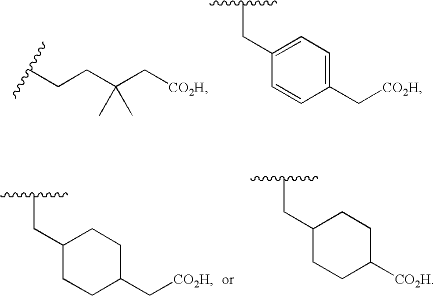

In another embodiment, R² is alkyl. In a particular embodiment, R² is ethyl.

In another embodiment, R⁴ is aryl optionally substituted with one or more alkyl groups, wherein said alkyl groups are optionally substituted with one or more F. In a particular embodiment, R⁴ is 3,5-ditrifluoromethylphenyl.

In yet another embodiment, R⁵ is 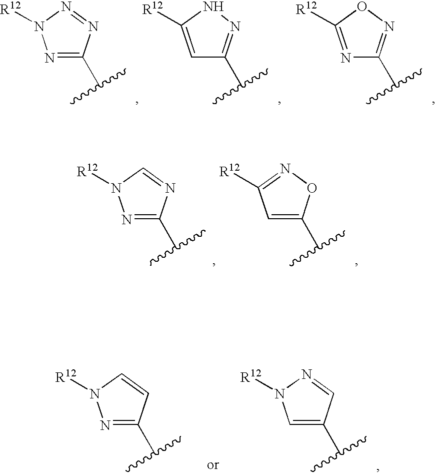
wherein R¹² is H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₘ-NR′R″, Zₙ-C(═O)NR′R″ or Zₘ-OR′.

In a particular embodiment, R⁵ is 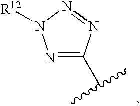
wherein R¹² is optionally H, alkyl, Zₙ-C(═O)OR′, Zₙ-CN, Zₘ-NR′R″, Zₙ-C(═O)NR′R″, Zₙ-cycloalkyl or Zₘ-OR′.

In yet another embodiment, R⁷ is F, Cl, Br, I, CF₃, OCF₂H or alkyl.

This invention further provides compounds having Formula II 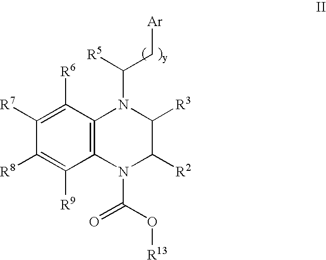
and metabolites, solvates, tautomers, resolved enantiomers, diastereomers, pharmaceutically acceptable salts and pharmaceutically acceptable prodrugs thereof, wherein:
R² is C₁-C₄ alkyl, C₂-C₄ allyl, C₂-C₄ alkenyl, C₂-C₄ alkynyl, C₁-C₄ heteroalkyl, C₂-C₄ heteroalkenyl, or C₂-C₄ heteroalkynyl, wherein said alkyl, alkenyl, alkynyl, heteroalkyl, heteroalkenyl and heteroalkynyl are optionally substituted with one or more groups independently selected from OR¹⁰ and SR¹⁰;
R³ is H, CF₃, Zₙ-NR¹¹R¹², Zₙ-NR¹⁰(C═O)R¹⁰R¹¹, Zₙ-SO₂R¹⁰, Zₙ-SOR¹⁰, Zₙ-SR¹⁰, Zₙ-OR¹⁰, Zₙ-(C═O)R¹⁰, Zₙ-(C═O)OR¹⁰, Zₙ-O(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar are optionally substituted with one or more groups independently selected from OR¹⁰ and SR¹⁰;
R⁵ is 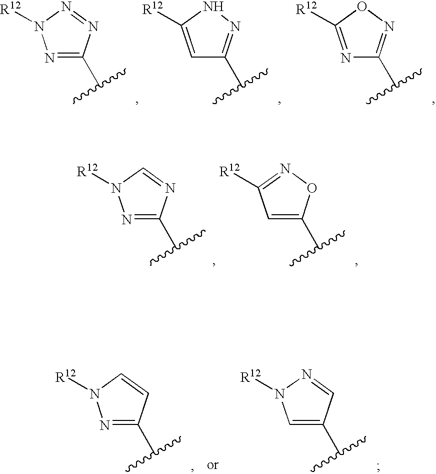
R⁶, R⁷, R⁸ and R⁹ are independently H, OH, F, Cl, Br, I, CF₃, OCF₃, OCF₂H, Zn-NR¹⁰R¹¹, Zₙ-(C═O)NR¹⁰R¹¹, Zₙ-SO₂R¹⁰, Zₙ-SOR¹⁰, Zₙ-SR¹⁰, Zₙ-OR¹⁰, Zₙ-(C═O)R¹⁰, Zₙ-(C═O)OR¹⁰, Zₙ-O—(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar,
or R⁶ and R⁷, R⁷ and R⁸, and/or R⁸ and R⁹ together with the atoms to which they are attached form a carbocyclic or heterocyclic ring, wherein said carbocyclic and heterocyclic rings are optionally substituted with one or more groups independently selected from alkyl and F;
R¹⁰ and R¹¹ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, cycloalkyl, heterocycloalkyl and Ar are optionally substituted with one or more groups independently selected from alkyl, OR′ and Ar,
or R¹⁰ and R¹¹ together with the atoms to which they are attached form a substituted or unsubstituted, saturated or partially unsaturated 5 or 6-membered heterocyclic ring;
R¹² is H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₘ-NR′R″, Zₙ-C(═O)NR′R″ or Zₘ-OR′;
R¹³ is C₁-C₄ alkyl, C₂-C₄ allyl, C₂-C₄ alkenyl, C₂-C₄ alkynyl, Zₙ-cycloalkyl, or Zₙ-Ar, wherein said alkyl, allyl, alkenyl, alkynyl, cycloalkyl and Ar are optionally substituted with one or more groups independently selected from OR′ and alkyl;
Ar is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, —OR′, SR′F, Cl, Br, I, CN and NO₂, wherein said alkyl is optionally substituted with one or more groups independently selected from C(═O)OR′, CN, NR′R″, C(═O)NR′R″, cycloalkyl, OR′, F and alkyl;
R′ and R″ are independently H or C₁-C₁₀ alkyl, wherein said alkyl is optionally substituted with one or more F;
Z is alkylene having from 1 to 4 carbons, or alkenylene or alkynylene each having from 2 to 4 carbons, wherein said alkylene, alkenylene and alkynylene are optionally substituted;
m is 1 or 2;
n is 0, 1, or 2; and
y is 0 or 1.

In one embodiment, R⁵ is 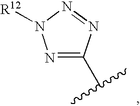
wherein R¹² is as defined herein. In a particular embodiment, R¹² is H, alkyl, Zₙ-C(═O)OR′, Zₙ-CN, Zₘ-NR′R″, Zₙ-C(═O)NR′R″, Zₙ-cycloalkyl or Zₘ-OR′.

In another embodiment, R² is alkyl. In a particular embodiment, R² is ethyl.

In another embodiment, Ar is aryl optionally substituted with one or more alkyl groups, wherein said alkyl groups are optionally substituted with one or more F.

In yet another embodiment, y is 0.

In another embodiment, R¹³ is alkyl or Zₙ-Ar.

This invention further provides compounds having Formula III 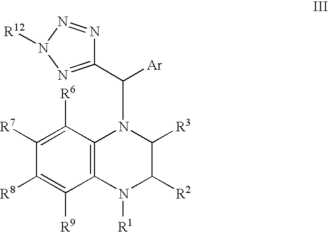
and metabolites, solvates, tautomers, resolved enantiomers, diastereomers, pharmaceutically acceptable salts and pharmaceutically acceptable prodrugs thereof, wherein:
R¹ is Zₙ-(C═O)OR¹⁰, Zₙ-(C═O)R¹⁰, Zₙ(C═O)Zₙ(C═O)OR¹⁰, Zₙ-NR¹⁰R¹¹, Zₙ-(C═O)NR¹⁰R¹¹, ZₙSOR¹⁰, Zₙ-SO₂R¹⁰, alkyl, allyl, alkenyl, alkynyl, heteroalkyl, heteroallyl, heteroalkenyl, heteroalkynyl, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, Zₙ-cycloalkyl, Zₙ-heterocycloalkyl and Zₙ-Ar are optionally substituted with one or more groups independently selected from F, Zₙ-COOR¹⁰, ZNOR¹⁰, Zₙ-NR¹⁰R¹¹, Zₙ-(C═O)NR¹⁰R¹¹, oxo and alkyl;
R² and R³ are independently H, OH, F, Cl, Br, I, CF₃, Zₙ-NR¹⁰R¹¹, Zₙ-NR¹⁰(C═O)R¹¹, Zₙ-SO₂R¹⁰, Zₙ-SOR¹⁰, Zₘ-SR¹⁰, Zₙ-OR¹⁰, Zₙ-(C═O)R¹⁰, Zₙ-(C═O)OR¹⁰, Zₙ-O(C═O)R¹⁰, alkyl, allyl, alkenyl, alkynyl, heteroalkyl, heteroallyl, heteroalkenyl, heteroalkynyl, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar are optionally substituted with one or more groups independently selected from OR¹⁰ and SR¹⁰,
or R¹ and R² together with the atoms to which they are attached form a substituted or unsubstituted, saturated or partially unsaturated 5 or 6-membered heterocyclic ring;
R⁶, R⁷, R⁸ and R⁹ are independently H, OH, F, Cl, Br, I, CF₃, OCF₃, OCF₂H, Zₙ-NR¹⁰R¹¹, Zₙ-(C═O)NR¹⁰R¹¹, Zₙ-SO₂R¹⁰, Zₙ-SOR¹⁰, Zₙ-SR¹¹, Zₙ-OR¹¹, Zₙ-(C═O)R¹⁰, Zₙ-(C═O)OR¹⁰, Zₙ-O—(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar,
or R⁶ and R⁷, R⁷ and R⁸, and/or R⁸ and R⁹ together with the atoms to which they are attached form a carbocyclic or heterocyclic ring, wherein said carbocyclic and heterocyclic rings are optionally substituted with one or more groups independently selected from alkyl and F;
R¹⁰ and R¹¹ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, cycloalkyl, heterocycloalkyl and Ar are optionally substituted with one or more groups independently selected from alkyl, OR′ and Ar,
or R¹⁰ and R¹¹ together with the atoms to which they are attached form a substituted or unsubstituted, saturated or partially unsaturated 5 or 6-membered heterocyclic ring;
R¹² is H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₘ-NR′R″, Zₙ-C(═O)NR′R″ or Zₘ-OR′;
Z is alkylene having from 1 to 4 carbons, or alkenylene or alkynylene each having from 2 to 4 carbons, wherein said alkylene, alkenylene and alkynylene are optionally substituted;
Ar is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, —OR′, SR′F, Cl, Br, I, CN and NO₂, wherein said alkyl is optionally substituted with one or more groups independently selected from C(═O)OR′, CN, —NR′R″, C(═O)NR′R″, cycloalkyl, OR′, F and alkyl;
R′ and R″ are independently H or C₁-C₁₀ alkyl, wherein said alkyl is optionally substituted with one or more F;
m is 1 or 2; and
n is 0, 1, or 2.

In one embodiment, R¹² is H, alkyl, Zₙ-C(═O)OR′, Zₙ-CN, Zₘ-NR′R″, Zₙ-C(═O)NR′R″, Zₙ-cycloalkyl or Zₘ-OR′.

In another embodiment, R² is alkyl. In a particular embodiment, R² is ethyl.

In another embodiment, Ar is aryl optionally substituted with one or more alkyl groups, wherein said alkyl groups are optionally substituted with one or more F.

The term “alkyl” as used herein refers to a saturated linear or branched-chain monovalent hydrocarbon radical of one to twelve carbon atoms, wherein the alkyl radical may be optionally substituted independently with one or more substituents described below. Examples of alkyl groups include, but are not limited to, methyl, ethyl, n-propyl, isopropyl, butyl, isobutyl, sec-butyl, tert-butyl, pentyl, isopentyl, tert-pentyl, hexyl, isohexyl, and the like.

“Alkylene” means a linear or branched saturated divalent hydrocarbon radical of one to twelve carbon atoms, e.g., methylene, ethylene, propylene, 2-methylpropylene, pentylene, and the like.

The term “alkenyl” refers to linear or branched-chain monovalent hydrocarbon radical of two to twelve carbon atoms containing at least one double bond, e.g., ethenyl, propenyl, and the like, wherein the alkenyl radical may be optionally substituted independently with one or more substituents described herein, and includes radicals having “cis” and “trans” orientations, or alternatively, “E” and “Z” orientations.

The term “alkenylene” refers to a linear or branched divalent hydrocarbon radical of two to twelve carbons containing at least one double bond, wherein the alkenylene radical may be optionally substituted independently with one or more substituents described herein. Examples include, but are not limited to, ethenylene, propenylene, and the like.

The term “alkynyl” refers to a linear or branched monovalent hydrocarbon radical of two to twelve carbon atoms containing at least one triple bond. Examples include, but are not limited to, ethynyl, propynyl, and the like, wherein the alkynyl radical may be optionally substituted independently with one or more substituents described herein.

The term “alkynylene” to a linear or branched divalent hydrocarbon radical of two to twelve carbons containing at least one triple bond, wherein the alkynylene radical may be optionally substituted independently with one or more substituents described herein.

The term “allyl” refers to a radical having the formula RC═CHCHR, wherein R is alkyl, alkenyl, alkynyl, cycloalkyl, heterocycloalkyl, aryl, heteroaryl, or any substituent as defined herein, wherein the allyl may be optionally substituted independently with one or more substituents described herein.

The term “cycloalkyl” refers to saturated or partially unsaturated cyclic hydrocarbon radical having from three to twelve carbon atoms, wherein the cycloalkyl may be optionally substituted independently with one or more substituents described herein. The term “cycloalkyl” further includes bicyclic and tricyclic cycloalkyl structures, wherein the bicyclic and tricyclic structures may include a saturated or partially unsaturated cycloalkyl fused to a saturated or partially unsaturated cycloalkyl or heterocycloalkyl ring or an aryl or heteroaryl ring. Examples of cycloalkyl groups include, but are not limited to, cyclopropyl, cyclobutyl, cyclopentyl, cyclohexyl, cycloheptyl, and the like.

The term “heteroalkyl” refers to saturated linear or branched-chain monovalent hydrocarbon radical of one to twelve carbon atoms, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroalkyl radical may be optionally substituted independently with one or more substituents described herein. The term “heteroalkyl” encompasses alkoxy and heteroalkoxy radicals.

The terms “heterocycloalkyl,” “heterocycle,” “hetercyclyl” and “heterocyclic ring” are used interchangeably herein and refer to a saturated or partially unsaturated carbocyclic radical of 3 to 8 ring atoms, wherein at least one of the carbon atoms in the ring is substituted with a heteroatom selected from N, O, or S, wherein one or more ring atoms may be optionally substituted independently with one or more substituents described below. The radical may be a carbon radical or heteroatom radical. The terms further include fused ring systems that include a heterocycle fused to a saturated or partially unsaturated cycloalkyl or heterocycloalkyl ring or an aryl or heteroaryl ring. Examples of heterocycloalkyl rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro moieties are also included within the scope of this definition. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl. The heterocycle groups herein are unsubstituted or, as specified, substituted in one or more substitutable positions with one or more substituents described herein. For example, such heterocycle groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆ alkenyl, C₂-C₆ alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

The term “heteroalkenyl” refers to linear or branched-chain monovalent hydrocarbon radical of two to twelve carbon atoms, containing at least one double bond, e.g., ethenyl, propenyl, and the like, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroalkenyl radical may be optionally substituted independently with one or more substituents described herein, and includes radicals having “cis” and “trans” orientations, or alternatively, “E” and “Z” orientations.

The term “heteroalkynyl” refers to a linear or branched monovalent hydrocarbon radical of two to twelve carbon atoms containing at least one triple bond. Examples include, but are not limited to, ethynyl, propynyl, and the like, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroalkynyl radical may be optionally substituted independently with one or more substituents described herein.

The term “heteroallyl” refers to radicals having the formula RC═CHCHR, wherein R is alkyl, alkenyl, alkynyl, cycloalkyl, heterocycloalkyl, aryl, heteroaryl, or any substituent as defined herein, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroallyl may be optionally substituted independently with one or more substituents described herein.

The term “aryl” refers to a monovalent aromatic carbocyclic radical having a single ring (e.g., phenyl), multiple rings (e.g., biphenyl), or multiple condensed rings in which at least one is aromatic, (e.g., 1,2,3,4-tetrahydronaphthyl, naphthyl, etc.), which are optionally mono-, di-, or trisubstituted independently with substituents such as halogen, lower alkyl, lower alkoxy, trifluoromethyl, aryl, heteroaryl, and hydroxy.

The term “heteroaryl” refers to a monovalent 5-, 6-, or 7-membered monovalent aromatic carbocyclic radical wherein at least one of the carbon atoms in the ring is substituted with a heteroatom selected from N, O, or S, and includes fused ring systems (at least one of which is aromatic) of 5-10 atoms. Examples of heteroaryl groups include, but are not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinzolinyl, quinoxalinyl, naphthyridinyl, and furopyridinyl. Spiro moieties are also included within the scope of this definition. Heteroaryl groups are optionally substituted with one or more substituents described herein.

The term “halo” represents fluoro, chloro, bromo or iodo.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of ester bioisosteres include, but are not limited to, 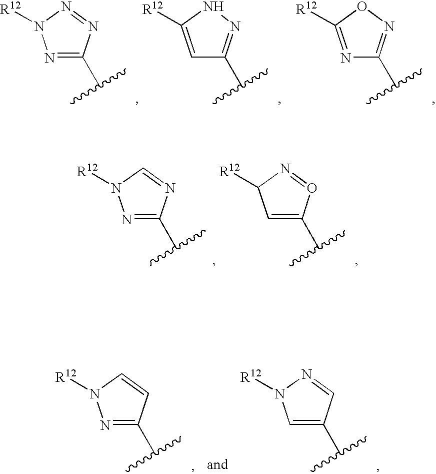
wherein R¹² is as defined herein.

In general, the various moieties or functional groups of the compounds of Formulas I and II may be optionally substituted by one or more substituents. Examples of substituents suitable for purposes of this invention include, but are not limited to, halo, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-OR′, Zₙ-NO₂, Zₙ—CN, Zₙ-CO₂R′, Zₙ-(C═O)R′, Zₙ-O(C═O)R′, Zₙ-OAr, Zₙ-SR′, Zₙ-SOR′, Zₙ-SO₂R″″, Zₙ-SAr Zₙ-SOAr, Zₙ-SO₂Ar, Zₙ-Ar, Zₙ-(C═O)NR″R′″, Zₙ-NR′R′″, NR′R′″, Zₙ-PO₃H₂, Zₙ-SO₃H₂, amine protecting groups, alcohol protecting groups, sulfur protecting groups, or acid protecting groups, where:
Z is alkylene having from 1 to 4 carbons, or alkenylene or alkynylene each having from 2 to 4 carbons, wherein said alkylene, alkenylene and alkynylene are optionally substituted;
n is zero or 1;
R′, R″ and R′″ are H, alkyl, allyl, alkenyl, alkynyl, heteroalkyl, heteroallyl, heteroalkenyl, heteroalkynyl, alkoxy, heteroalkoxy, Zₙ-cycloalkyl or Zₙ-heterocycloalkyl, and R″″ is alkyl, allyl, alkenyl, alkynyl, heteroalkyl, heteroallyl, heteroalkenyl, heteroalkynyl, alkoxy, heteroalkoxy, Zₙ-cycloalkyl, or Zₙ-heterocycloalkyl; and
Ar is aryl or heteroaryl;
wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Ar, R′, R″, and R′″ and R″″ may be substituted or unsubstituted.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers, diastereomers mixtures, racemic or otherwise, thereof. Accordingly, this invention also includes all such isomers, including diastereomeric mixtures and pure enantiomers of the compounds of Formulas I-III. Diastereomeric mixtures can be separated into their individual diastereomers on the basis of their physical chemical differences by methods known to those skilled in the art, for example, by chromatography or fractional crystallization. Enantiomers can be separated by converting the enantiomer mixture into a diastereomeric mixture by reaction with an appropriate optically active compound (e.g., alcohol), separating the diastereomers and converting (e.g., hydrolyzing) the individual diastereomers to the corresponding pure enantiomers. The methods for the determination of stereochemistry and the separation of stereoisomers are well known in the art (see discussion in Chapter 4 of “Advanced Organic Chemistry”, 4th edition, J. March, John Wiley and Sons, New York, 1992).

In addition to compounds of the Formula I-III, the invention also includes solvates, pharmaceutically acceptable prodrugs, pharmaceutically active metabolites, and pharmaceutically acceptable salts of such compounds.

The term “solvate” refers to an aggregate of a molecule with one or more solvent molecules.

A “pharmaceutically acceptable prodrug” is a compound that may be converted under physiological conditions or by solvolysis to the specified compound or to a pharmaceutically acceptable salt of such compound. Prodrugs include compounds wherein an amino acid residue, or a polypeptide chain of two or more (e.g., two, three or four) amino acid residues (i.e., peptides) is covalently joined through an amide or ester bond to a free amino, hydroxy or carboxylic acid group of compounds of the present invention. Amino acid residues include, but are not limited to, the 20 naturally occurring amino acids commonly designated by three letter symbols and also includes 4-hydroxyproline, hydroxylysine, demosine, isodemosine, 3-methylhistidine, norvaline, beta-alanine, gamma-aminobutyric acid, cirtulline, homocysteine, homoserine, ornithine and methionine sulfone. One example of a prodrug of this invention is a compound of Formula I, II or III covalently joined to a phosphate residue. Another example of a prodrug of this invention is a compound of Formula I, II or III covalently joined to a valine residue or an alanine-alanine dipeptide.

Additional types of prodrugs are also encompassed. For instance, free carboxyl groups can be derivatized as amides or alkyl esters. As another example, compounds of this invention comprising free hydroxy groups may be derivatized as prodrugs by converting the hydroxy group into groups such as, but not limited to, phosphate ester, hemisuccinate, dimethylaminoacetate, or phosphoryloxymethyloxycarbonyl groups, as outlined in Advanced Drug Delivery Reviews, (1996) 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including, but not limited to, ether, amine and carboxylic acid functionalities, or where the acyl group is an amino acid ester as described above, are also encompassed. Prodrugs of this type are described in J. Med. Chem., (1996) 39, 10. More specific examples include replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

Free amines can also be derivatized as amides, sulfonamides or phosphonamides. All of these prodrug moieties may incorporate groups including, but not limited to, ether, amine and carboxylic acid functionalities. For example, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY wherein Y is H, (C₁-C₆)alkyl or benzyl, —C(OY₀)Y₁ wherein Y₀ is (C₁-C₄) alkyl and Y₁ is (C₁-C₆)alkyl, carboxy(C₁-C₆)alkyl, amino(C₁-C₄)alkyl or mono-N- or di-N,N-(C₁-C₆)alkylaminoalkyl, —C(Y₂)Y₃ wherein Y₂ is H or methyl and Y₃ is mono-N- or di-N,N-(C₁-C₆)alkylamino, morpholino, piperidin-1-yl or pyrrolidin-1-yl.

Prodrugs of a compound may be identified using routine techniques known in the art. Various forms of prodrugs are known in the art. For examples of such prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, (1992) 8:1-38; d) H. Bundgaard, et al., J. Pharmaceutical Sciences, (1988) 77:285; and e) N. Kakeya, et al., Chem. Pharm. Bull., (1984) 32:692, each of which is specifically incorporated herein by reference.

A “metabolite” is a pharmacologically active product produced through in vivo metabolism of a specified compound or salt or prodrug thereof. Such products may result for example from the oxidation, reduction, hydrolysis, amidation, deamidation, esterification, deesterification, enzymatic cleavage, and the like, of the administered compound. The invention also includes products produced by a process comprising contacting a compound of this invention with a mammal for a period of time sufficient to yield a metabolic product thereof.

Metabolites of a compound may be identified using routine techniques known in the art. For example, metabolite products typically are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

A “pharmaceutically acceptable salt” is a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Examples of pharmaceutically acceptable salts include those salts prepared by reaction of the compounds of the present invention with a mineral or organic acid or an inorganic base, such salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyn-1,4-dioates, hexyne-1,6-dioates, benzoates, chlorobenzoates, methylbenzoates, dinitromenzoates, hydroxybenzoates, methoxybenzoates, phthalates, sulfonates, xylenesulfonates, pheylacetates, phenylpropionates, phenylbutyrates, citrates, lactates, β-hydroxybutyrates, glycollates, tartrates, methanesulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

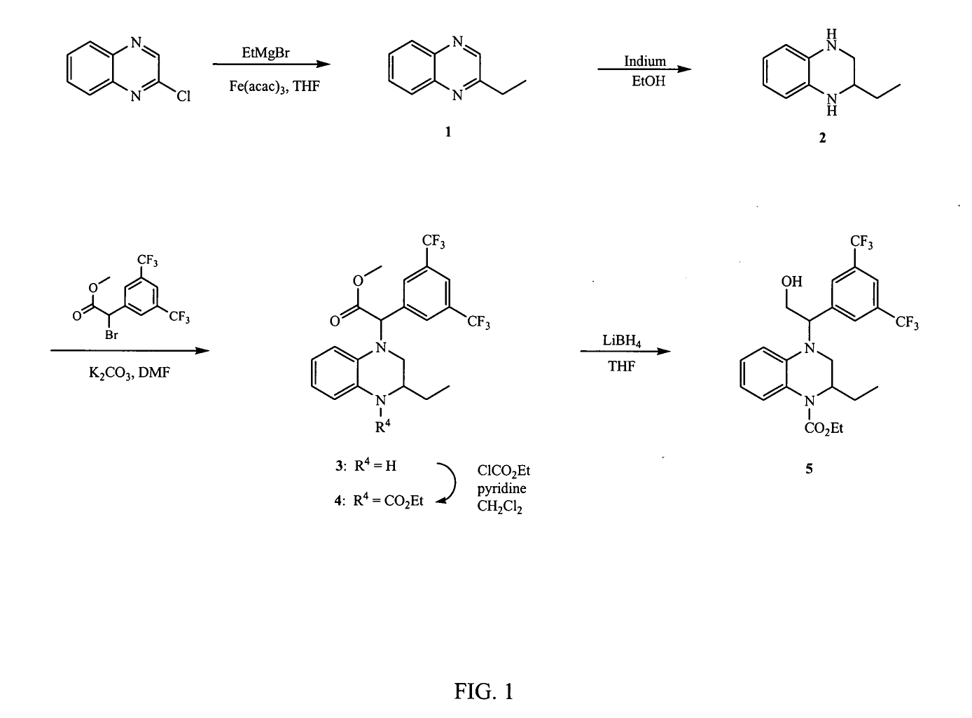

The inventive compounds may be prepared using the reaction routes and synthesis schemes as described below, employing the techniques available in the art using starting materials that are readily available. FIGS. 1-5 show examples of the synthesis of specific compounds of the general Formula I-III.

This invention further includes a compound of the general Formula I-III for use as a medicament for the treatment of an abnormal cell growth condition in a human or animal. Also included in this invention is use of a compound of the general Formula I-III in the manufacture of a medicament for the treatment of a CETP-mediated condition in a human or animal.

The compounds and pharmaceutical compositions of this invention are useful for treating a disorder or condition selected from cerebrovascular disease, coronary artery disease, hypertension, ventricular dysfunction, cardiac arrhythmia, pulmonary vascular disease, peripheral vascular disease, reno-vascular disease, renal disease, splanchnic vascular disease, vascular hemostatic disease, diabetes, inflammatory disease, autoimmune disorders and other systemic disease indications, immune function modulation, pulmonary disease, anti-oxidant disease, sexual dysfunction, cognitive dysfunction, schistosomiasis and cancer in a mammal, comprising administering to said mammal a therapeutically effective amount of a cholesteryl ester transfer protein (CETP) inhibitor or a pharmaceutically acceptable salt thereof, in amounts that render the active agents effective in the treatment of said disorder or condition.

The term “cerebrovascular disease” as used herein includes, but is not limited to, ischemic attacks (e.g., transient), ischemic stroke (transient), acute stroke, cerebral apoplexy, hemorrhagic stroke, neurologic deficits post-stroke, first stroke, recurrent stroke, shortened recovery time after stroke and provision of thrombolytic therapy for stroke. Preferable patient populations include patients with or without pre-existing stroke or coronary heart disease.

The term “coronary artery disease” includes, but is not limited to, atherosclerotic plaque (e.g., prevention, regression, stabilization), vulnerable plaque (e.g., prevention, regression, stabilization), vulnerable plaque area (reduction), arterial calcification (e.g., calcific aortic stenosis), increased coronary artery calcium score, dysfunctional vascular reactivity, vasodilation disorders, coronary artery spasm, first myocardial infarction, myocardia re-infarction, ischemic cardiomyopathy, stent restenosis, PTCA restenosis, arterial restenosis, coronary bypass graft restenosis, vascular bypass restenosis, decreased exercise treadmill time, angina pectoris/chest pain, unstable angina pectoris, exertional dyspnea, decreased exercise capacity, ischemia (reduce time to), silent ischemia (reduce time to), increased severity and frequency of ischemic symptoms, reperfusion after thrombolytic therapy for acute myocardial infarction.

The term “hypertension” includes, but is not limited to, lipid disorders with hypertension, systolic hypertension and diastolic hypertension.

The term “diabetes” includes, but is not limited to, type II diabetes, Syndrome X, Metabolic syndrome, lipid disorders associated with insulin resistance, non-insulin dependent diabetes, microvascular diabetic complications, reduced nerve conduction velocity, reduced or loss of vision, diabetic retinopathy, increased risk of amputation, decreased kidney function, kidney failure, metabolic syndrome, insulin resistance syndrome, pluri-metabolic syndrome, central adiposity (visceral) (upper body), diabetic dyslipidemia, decreased insulin sensitization, diabetic retinopathy/neuropathy, diabetic nephropathy/micro and macro angiopathy and micro/macro albuminuria, dyslipidemia, diabetic cardiomyopathy, diabetic gastroparesis, obesity, increased hemoglobin glycoslation, impaired renal and hepatic function.

The term “cognitive dysfunction” includes, but is not limited to, dementia secondary to atherosclerosis, transient cerebral ischemic attacks, neurodegeneration, neuronal deficient, and delayed onset or procession of Alzheimer's disease.

The term “ventricular dysfunction” includes, but is not limited to, systolic dysfunction, diastolic dysfunction, heart failure, congestive heart failure, dilated cardiomyopathy, idiopathic dilated cardiomyopathy, and non-dilated cardiomopathy.

The term “cardiac arrhythmia” includes, but is not limited to, atrial arrhythmias, supraventricular arrhythmias, ventricular arrhythmias and sudden death syndrome.

The term “pulmonary vascular disease” includes, but is not limited to, pulmonary hypertension and pulmonary embolism.

The term “peripheral vascular disease” includes, but is not limited to, peripheral vascular disease and claudication

The term “reno-vascular/renal disease” includes, but is not limited to, renal vascular diseases, renal hypertension and renal arterial stenosis.

The term “splanchnic vascular disease” includes, but is not limited to, ischemic bowel disease.

The term “vascular hemostatic disease” includes, but is not limited to, deep venous thrombosis, vaso-occlusive complications of sickle cell anemia, varicose veins, pulmonary embolism, transient ischemic attacks, embolic events, including stroke, in patients with mechanical heart valves, embolic events, including stroke, in patients with right or left ventricular assist devices, embolic events, including stroke, in patients with intra-aortic balloon pump support, embolic events, including stroke, in patients with artificial hearts, embolic events, including stroke, in patients with cardiomyopathy, embolic events, including stroke, in patients with atrial fibrillation or atrial flutter.

The terms “inflammatory disease,” “autoimmune disorders” and other systemic diseases include, but are not limited to, multiple sclerosis, rheumatoid arthritis, osteoarthritis, irritable bowel syndrome, irritable bowel disease, Crohn's disease, colitis, vasculitis, lupus erythematosis, sarcoidosis, amyloidosis, and apoptosis.

The term “pulmonary disease” includes, but is not limited to, pulmonary fibrosis, emphysema, obstructive lung disease, chronic hypoxic lung disease, antioxidant deficiencies, hyper-oxidant disorders and asthma.

The term “immune function disease” includes, but is not limited to, transplant vasculopathy, solid organ transplant rejection, transplant rejection, impaired toxin sequestration/removal, elevated levels of CXC chemokines, interleukins including interleukin-1, 6 and 8, neutrophil-activating protein-2 (NAP-2), melanoma growth stimulatory activity protein (MGSA), and elevated levels of CC chemokines, RANTES, MIP-1 alpha and beta, MCP-1, -2, -3, -4, -5 Eotaxin-1, -2, -3, C-reactive protein including highly sensitive C-reactive protein and TNF-α.

The term “anti-oxidant disease” as used herein includes, but is not limited to, aging, mortality, apoptosis and increased oxidative stress

The term “sexual dysfunction” includes, but is not limited to, male sexual dysfunction, erectile dysfunction and female sexual dysfunction.

The term “cognitive dysfunction”, as used herein includes, but is not limited to, dementia secondary to atherosclerosis, neurodegeneration, neuronal deficient, and delayed onset or procession of Alzheimer's disease.

Additionally, CETP compounds and the combinations included herewith are also useful for neurodegenerative diseases such as Parkinson's, Huntington's disease, amyloid deposition and amylotrophic lateral sclerosis.

The term “cancer” as used herein includes, but not limited to, resistance to chemotherapy, unregulated cell growth, hyperplasia (e.g., benign prostatic hyperplasia) and any of a number of abnormal multiplication or increase in the number of normal cells in normal arrangement in a tissue. The compounds and combinations included herein are also useful for cancer prevention.

The CETP inhibitors and combinations thereof included herein are useful for reducing global cardiovascular risk and global risk scores.

The CETP inhibitors are also useful for modulation of plasma and or serum or tissue lipids or lipoproteins, such as HDL subtypes (e.g., increase, including pre-beta HDL, HDL-1,-2 and, 3 particles) as measured by precipitation or by apo-protein content, size, density, NMR profile, FPLC and charge and particle number and its constituents; and LDL subtypes (including LDL subtypes e.g., decreasing small dense LDL, oxidized LDL, VLDL, apo(a) and Lp(a) as measured by precipitation, or by apo-protein content, size density, NMR profile, FPLC and charge; IDL and remnants (decrease); phospholipids (e.g., increase HDL phospholipids); apo-lipoproteins (increase A-I, A-II, A-IV, decrease total and LDL B-100, decrease B-48, modulate C-II, C-III, E, J); paraoxonase (increase, anti-oxidant effects, anti-inflammatory effects); decrease post-prandial (hyper)lipemia; decrease triglycerides; decrease non-HDL; elevate HDL in subjects with low HDL due to increased CETP mass or activity and optimize and increase ratios of HDL to LDL (e.g., greater than 0.25).

Therapeutically effective amounts of the compounds of the invention may be used to treat diseases mediated by modulation or regulation of protein kinases. An “effective amount” is intended to mean that amount of compound that, when administered to a mammal in need of such treatment, is sufficient to effect treatment for a disease mediated by CETP. Thus, for example, a therapeutically effective amount of a compound selected from Formula I, II or III or a salt, active metabolite or prodrug thereof, is a quantity sufficient to modulate, regulate, or inhibit the activity of one or more protein kinases such that a disease condition which is mediated by that activity is reduced or alleviated.

The amount of a given agent that will correspond to such an amount will vary depending upon factors such as the particular compound, disease condition and its severity, the identity (e.g., weight) of the mammal in need of treatment, but can nevertheless be routinely determined by one skilled in the art. “Treating” is intended to mean at least the mitigation of a disease condition in a mammal, such as a human, that is affected, at least in part, by CETP and includes, but is not limited to, preventing the disease condition from occurring in a mammal, particularly when the mammal is found to be predisposed to having the disease condition but has not yet been diagnosed as having it; modulating and/or inhibiting the disease condition; and/or alleviating the disease condition.

In order to use a compound of the Formula I, II or III, or a pharmaceutically acceptable salt or in vivo cleavable prodrug thereof, for the therapeutic treatment (including prophylactic treatment) of mammals including humans, it is normally formulated in accordance with standard pharmaceutical practice as a pharmaceutical composition. According to this aspect of the invention there is provided a pharmaceutical composition that comprises a compound of the Formula I, II or III, or a pharmaceutically acceptable salt or in vivo cleavable prodrug thereof, as defined hereinbefore in association with a pharmaceutically acceptable diluent or carrier.

To prepare the pharmaceutical compositions according to one embodiment of this invention, a therapeutically or prophylactically effective amount of a compound of Formula I, II or III, or a pharmaceutically acceptable salt, solvate, metabolite or prodrug thereof (alone or together with an additional therapeutic agent) is intimately admixed with a pharmaceutically acceptable carrier according to conventional pharmaceutical compounding techniques to produce a dose. A carrier may take a wide variety of forms depending on the form of preparation desired for administration, e.g., oral or parenteral. Examples of suitable carriers include any and all solvents, dispersion media, adjuvants, coatings, antibacterial and antifungal agents, isotonic and absorption delaying agents, sweeteners, stabilizers (to promote long term storage), emulsifiers, binding agents, thickening agents, salts, preservatives, solvents, dispersion media, coatings, antibacterial and antifungal agents, isotonic and absorption delaying agents, flavoring agents, and miscellaneous materials such as buffers and absorbents that may be needed in order to prepare a particular therapeutic composition. The use of such media and agents with pharmaceutically active substances is well known in the art. Except insofar as any conventional media or agent is incompatible with a compound of Formula I, II or III, its use in the therapeutic compositions and preparations is contemplated. Supplementary active ingredients can also be incorporated into the compositions and preparations as described herein.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing or as a suppository for rectal dosing). For example, compositions intended for oral use may contain, for example, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The amount of a compound of this invention that is combined with one or more excipients to produce a single dosage form will necessarily vary depending upon the subject treated, the severity of the disorder or condition, the rate of administration, the disposition of the compound and the discretion of the prescribing physician. However, an effective dosage is in the range of about 0.001 to about 100 mg per kg body weight per day, preferably about 1 mg/kg/day to about 35 mg/kg/day, in single or divided doses. For a 70 kg human, this would amount to about 0.07 to 2.45 g/day, preferably about 0.05 to about 1.0 g/day. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In one aspect of this invention, the compounds of this invention or pharmaceutical salts or prodrugs thereof may be formulated into pharmaceutical compositions for administration to animals or humans to treat or prevent a CETP-mediated condition. The term “CETP condition” as used herein means any disease or other deleterious condition in which CETP is known to play a role.

In another embodiment of the invention, an article of manufacture, or “kit”, containing materials useful for the treatment of the disorders described above is provided. In one embodiment, the kit comprises a container comprising a composition of Formula I, II or III or a pharmaceutically acceptable salt or prodrug thereof, or a pharmaceutical composition comprising said compound. The kit may further comprise a label or package insert on or associated with the container. Suitable containers include, for example, bottles, vials, syringes, blister pack, etc. The container may be formed from a variety of materials such as glass or plastic. The container holds a compound of Formula I, II or III or a formulation thereof which is effective for treating the condition and may have a sterile access port (for example, the container may be an intravenous solution bag or a vial having a stopper pierceable by a hypodermic injection needle). The label or package insert indicates that the composition is used for treating the condition of choice, such as cancer. In one embodiment, the label or package inserts indicates that the composition comprising a compound of Formula I, II or III can be used to treat a CETP-mediated condition. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of Formula I, II or III and, if present, the second pharmaceutical formulation. For example, if the kit comprises a first composition comprising a compound of Formula I, II or III and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a compound of Formula I, II or III, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, an article of manufacture may comprise (a) a first container with a compound of Formula I, II or III contained therein; and optionally (b) a second container with a second pharmaceutical formulation contained therein, wherein the second pharmaceutical formulation comprises a second CETP inhibitor. Alternatively, or additionally, the article of manufacture may further comprise a third container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises a composition of Formula I, II or III and a second therapeutic agent, the kit may comprise a container for containing the separate compositions such as a divided bottle or a divided foil packet; however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare a number of other CETP inhibitors of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

### EXAMPLES

In the examples described below, unless otherwise indicated all temperatures are set forth in degrees Celsius. Reagents were purchased from commercial suppliers such as Aldrich Chemical Company, Lancaster, TCI or Maybridge, and were used without further purification unless otherwise indicated. Tetrahydrofuran (THF), N,N-dimethylformamide (DMF), dichloromethane (DCM), toluene, dioxane and 1,2-difluoroethane were purchased from Aldrich in Sure seal bottles and used as received.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

¹H-NMR spectra were recorded on a Bruker instrument operating at 300 MHz or on a Varian instrument operating at 400 MHz. ¹H-NMR spectra were obtained as CDCl₃ solutions (reported in ppm), using chloroform as the reference standard (7.25 ppm). Other NMR solvents were used as needed. When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

### Example 1

### Synthesis of 4-[1-(3,5-Bis-trifluoromethylphenyl)-2-hydroxyethyl]-2-ethyl-3,4-dihydro-2H-quinoxaline-1-carboxylic acid ethyl ester (5)

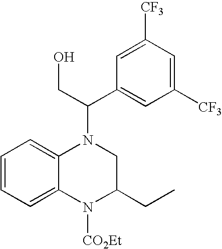

The synthesis of compound (5) according to Example 1 is illustrated in FIG. 1.

Step A: 2-Ethyl-quinoxaline (1): To a flame dried, nitrogen purged 500 mL flask was added 2-chloroquinoxaline (2.30 g, 14.0 mmol) and Fe(acac)₃ (0.25 g, 0.70 mmol). The solids were diluted with THF (100 mL) and NMP (8 mL). A solution of EtMgBr (2.23 g, 16.8 mmol) was added dropwise over 10 minutes. The red solution turned dark brown. After 20 minutes, the reaction was diluted with ether (100 mL). The flask was cooled to 0° C. in an ice bath and 1N HCl (30 mL) was added cautiously. After 10 minutes of stirring, water (100 mL) was added and the layers separated. The ether layer was washed with brine (100 mL), dried over Na₂SO₄, and concentrated. The crude oil was purified by column chromatography, (Biotage 40m, 10% EtOAc/hexanes) to give 2-ethylquinoxaline (1) as a light yellow oil (1.50 g, 68%).

Step B: 2-Ethyl-1,2,3,4-tetrahydroquinoxaline (2): Saturated NH₄Cl (3 mL) and Indium powder (9.8 g, 85 mmol) were added to a solution of 2-ethylquinoxaline (1.50 g, 9.48 mmol) in EtOH (48 mL). The reaction was heated to reflux for 14 hours. The cooled reaction mixture was diluted with water (50 mL) and filtered through celite 545. The aqueous layer was neutralized to pH 10 with 10% NaOH, then extracted twice with CH₂Cl₂ (200 mL). The combined organic layers were washed with brine (100 mL), dried over Na₂SO₄, and concentrated to give 2-ethyl-1,2,3,4-tetrahydroquinoxaline (2) as a yellow solid (1.54 g, 100%).

Step C: (3,5-Bis-trifluoromethylphenyl)-(3-ethyl-3,4-dihydro-2H-quinoxalin-1-yl)-acetic acid methyl ester (3): Potassium carbonate (0.432 g, 3.12 mmol), 2-ethyl-1,2,3,4-tetrahydroquinoxaline (0.507 g, 3.12 mmol), and Me₄NI (0.115 g, 0.312 mmol) were weighed into a 25 mL flask and placed under a nitrogen atmosphere. Dimethylformamide (25 mL) was added, followed by (3,5-bis-trifluoromethylphenyl)-bromoacetic acid methyl ester (1.140 g, 3.12 mmol). The reaction was stirred at room temperature for 2 hours. The reaction mixture was poured into water (150 mL) and extracted twice with EtOAc (100 mL). The combined organic layers were washed three times with brine (100 mL), dried over Na₂SO₄, and concentrated. The resulting oil was purified by column chromatography (Biotage 40m, 1:1 CH₂Cl₂/hexanes, then 100% CH₂Cl₂) to provide a mixture of diastereomers (1:1) of (3,5-bis-trifluoromethylphenyl)-(3-ethyl-3,4-dihydro-2H-quinoxalin-1-yl)-acetic acid methyl ester (3) as a yellow solid (0.618 g, 44%). ¹H NMR δ 0.73-0.87 (m, 3H), 1.29-1.58 (m, 2H), 2.80-2.83 (dd, 0.5H), 2.95-3.02 (m, 0.5H), 3.13-3.23 (m, 1.5H), 3.33-3.37 (dd, 0.5H), 3.83-3.85 (d, 3H), 5.66-5.67 (d, 1H), 6.53-6.74 (m, 4H), 7.73-7.94 (m, 3H).

Step D: 4-[(3,5-Bis-trifluoromethylphenyl)-methoxycarbonylmethyl]-2-ethyl-3,4-dihydro-2H-quinoxaline-1-carboxylic acid ethyl ester (4): To a solution of (3,5-bis-trifluoromethylphenyl)-(3-ethyl-3,4-dihydro-2H-quinoxalin-1-yl)-acetic acid methyl ester (0.400 g, 0.896 mmol) in CH₂Cl₂ (40 mL) was added pyridine (0.106 g, 1.34 mmol) and ethyl chloroformate (0.146 g, 1.34 mmol). The reaction was stirred at room temperature for 45 minutes. HPLC showed the reaction to be complete and the two product diastereomers were present (1:1 ratio). The reaction was washed with 1N HCl (30 mL), saturated NaHCO₃ (30 mL), dried over Na₂SO₄, and concentrated. The resulting oil was purified by column chromatography (Biotage 40s, 3:2 CH₂Cl₂/hexanes, then 4:1 CH₂Cl₂/hexanes) to provide two diastereomers of 4-[(3,5-bis-trifluoromethylphenyl)-methoxycarbonylmethyl]-2-ethyl-3,4-dihydro-2H-quinoxaline-1-carboxylic acid ethyl ester (4) as viscous yellow oils. (Higher R_{f} by TLC, 29.8 mg, 6%) and (Lower R_{f} by TLC, 53.3 mg, 11%). ¹H NMR (CDCl₃) (Higher R_{f} by TLC) δ 0.85-0.92 (t, 3H, J=7.04 Hz), 1.28-1.33 (t, 3H, J=7.04), 1.42-1.52 (m, 2H), 3.16-3.20 (dd, 1H, J=4.70 Hz), 3.26-3.29 (dd, 1H, J=1.56 Hz), 3.83 (s, 3H), 4.16-4.29 (m, 2H), 4.48-4.50 (m, 1H), 5.66 (s, 1H), 6.60-6.62 (d, 1H, J=8.61), 6.78-6.82 (dd, 1H, J=7.82 Hz), 7.01-7.07 (dd, 1H, J=8.61 Hz), 7.50-7.52 (br d, 1H), 7.77 (s, 2H), 7.88 (s, 1H). ¹H NMR (Lower R_{f} by TLC) δ 0.72-0.76 (t, 3H), 1.19-1.23 (m, 1H), 1.29-1.34 (t, 3H), 1.43-1.50 (m, 1H), 2.85-2.87 (dd, 1H), 3.44-3.48 (dd, 1H), 3.84 (s, 3H), 4.19-4.30 (m, 2H), 4.45 (m, 1H), 5.82 (s, 1H), 6.76-6.84 (m, 2H), 7.03 (t, 1H), 7.66 (br d, 1H), 7.77 (s, 2H), 7.90 (s, 1H).

Step E: 4-[1-(3,5-Bis-trifluoromethylphenyl)-2-hydroxyethyl]-2-ethyl-3,4-dihydro-2H-quinoxaline-1-carboxylic acid ethyl ester (5): A mixture of diastereomers of 4-[(3,5-bis-trifluoromethylphenyl)-methoxycarbonylmethyl]-2-ethyl-3,4-dihydro-2H-quinoxaline-1-carboxylic acid ethyl ester was stirred in THF (5 mL) under a nitrogen atmosphere. A solution of LiBH₄ (0.053 mL, 0.11 mmol, 2.0 M solution in THF) was added and the reaction stirred at room temperature for 2 hours. The reaction was poured into water (50 mL) and extracted twice with EtOAc (25 mL). The organic layers were washed with brine (50 mL), dried over Na₂SO₄, and concentrated. The resulting oil was purified by column chromatography (Biotage 12m (1:20 EtOAc/DCM) to obtain (3,5-bis-trifluoromethylphenyl)-(3-ethyl-3,4-dihydro-2H-quinoxalin-1-yl)-acetic acid methyl ester (5) as a colorless film (0.400 g, 0.896 mmol). ¹H NMR (CDCl₃) (Higher R_{f} by TLC): δ 0.93 (t, 3H), 1.34 (t, 3H), 1.45 (m, 2H), 1.78 (t, 1H), 3.16-3.29 (m, 2H), 4.24-4.30 (m, 4H), 4.55 (br m, 1H), 5.22 (t, 1H), 6.76 (dd, 2H), 7.02 (t, 1H), 7.52 (br d, 1H), 7.74 (s, 2H), 7.81 (s, 1H). ¹H NMR (Lower R_{f} by TLC): δ 0.80 (t, 3H), 1.22-1.33 (m, 4H), 1.43-1.52 (m, 1H), 1.80 (t, 1H), 3.18 (d, 1H), 3.50 (dd, 1H), 4.10-4.33 (m, 4H), 4.51 (br m, 1H), 5.16 (t, 1H), 6.73 (dd, 2H), 7.00 (t, 1H), 7.52 (br s, 1H), 7.83 (s, 3H).

### Example 2

### Synthesis of 4-[(3,5-bis-trifluoromethylphenyl)-(2-methyl-2H-tetrazol-5-yl)-methyl]-2-ethyl-3,4-dihydro-2H-quinoxaline-1-carboxylic acid ethyl ester (10)

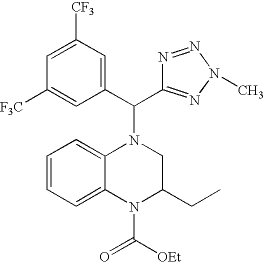

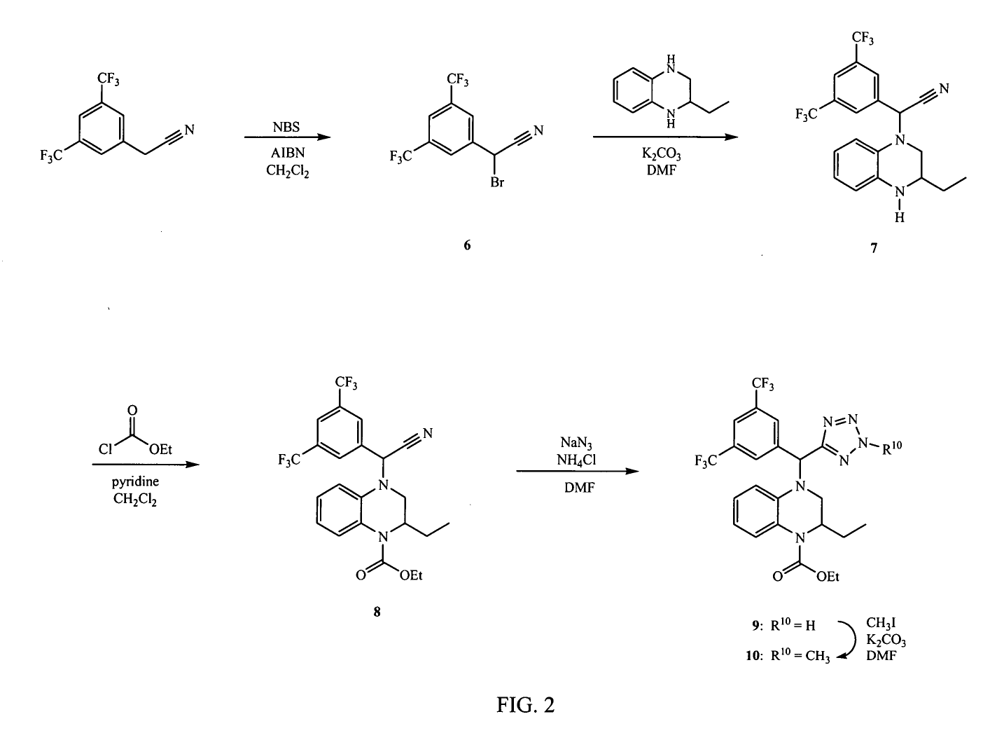

The synthesis of compound (10) according to Example 2 is illustrated in FIG. 2.

Step A: (3,5-Bis-trifluoromethylphenyl)-bromoacetonitrile (6): To a solution of (3,5-bis-trifluoromethylphenyl)-acetonitrile (4.66 g, 18.4 mmol) in CCl₄ (50 mL) under a nitrogen atmosphere was added NBS (3.93 g, 22.1 mmol) and AIBN (15.1 mg, 0.0930 mmol). The reaction was heated to reflux for 4 hours. The reaction was cooled to room temperature and diluted with CH₂Cl₂ (150 mL). The organic layer was washed with water (50 mL), then brine (50 mL), dried over Na₂SO₄, and concentrated. The resulting oil was purified by column chromatography (Biotage 60m (2:1 hexanes/CH₂Cl₂) to obtain (3,5-bis-trifluoromethylphenyl)-bromoacetonitrile (6) as a colorless film (1.4 g, 4.2 mmol, 23%).

Step B: (3,5-Bis-trifluoromethylphenyl)-(3-ethyl-3,4-dihydro-2H-quinoxalin-1-yl)-acetonitrile (7): To a solution of (3,5-bis-trifluoromethylphenyl)-bromoacetonitrile (0.449 g, 1.35 mmol) in DMF (4 mL) under a nitrogen atmosphere was added K₂CO₃ (0.280 g, 2.02 mmol) and 2-ethyl-1,2,3,4-tetrahydroquinoxaline (0.210 g, 1.350 mmol). The reaction was stirred at room temperature for 2 hours and then was poured into water (30 mL) and extracted twice with EtOAc (30 mL). The combined organic layers were washed twice with brine (50 mL), dried over Na₂SO₄, and concentrated. The resulting oil was purified by flash chromatography (1:2 hexanes/CH₂Cl₂) to obtain (3,5-bis-trifluoromethylphenyl)-(3-ethyl-3,4-dihydro-2H-quinoxalin-1-yl)-acetonitrile (7) as a colorless oil (0.277 g, 0.670 mmol, 50%).

Step C: 4-[(3,5-Bis-trifluoromethylphenyl)-cyanomethyl]-2-ethyl-3,4-dihydro-2H-quinoxaline-1-carboxylic acid ethyl ester (8): To a solution of (3,5-bis-trifluoromethylphenyl)-(3-ethyl-3,4-dihydro-2H-quinoxalin-1-yl)-acetonitrile (0.277 g, 0.670 mmol) in CH₂Cl₂ (10 mL) was added pyridine (0.0636 g, 0.804 mmol) and ethyl chloroformate (0.0873, 0.804 mmol). The reaction was stirred at room temperature for 45 minutes. The reaction was washed with 1N HCl (20 mL), saturated NaHCO₃ (20 mL), dried over Na₂SO₄, and concentrated. The resulting oil was purified by flash chromatography (1:1 CH₂Cl₂/hexanes, then 4:1 CH₂Cl₂/hexanes) to provide a mixture (1:1) of the two diastereomers of 4-[(3,5-bis-trifluoromethylphenyl)-cyanomethyl]-2-ethyl-3,4-dihydro-2H-quinoxaline-1-carboxylic acid ethyl ester (8) as a colorless oil (0.168 g, 0.346 mmol, 52%).

Step D: 4-[(3,5-Bis-trifluoromethylphenyl)-(2H-tetrazol-5-yl)-methyl]-2-ethyl-3,4-dihydro-2H-quinoxaline-1-carboxylic acid ethyl ester (9): To a solution of 4-[(3,5-bis-trifluoromethylphenyl)-cyanomethyl]-2-ethyl-3,4-dihydro-2H-quinoxaline-1-carboxylic acid ethyl ester (56 mg, 0.12 mmol) in DMF (5 mL) was added NaN₃ (37.5 mg, 0.577 mmol) and NH₄Cl (30.9 mg, 0.577 mmol). The reaction was heated to 75° C. for 10 hours. The reaction was poured into saturated NaHCO₃ (30 mL) and extracted three times with CH₂Cl₂ (15 mL). The combined organic layers were washed with brine (20 mL), dried over Na₂SO₄, and concentrated. The resulting oil was purified by column chromatography (Biotage 12m, 100% CH₂Cl₂, then 5:1 CH₂Cl₂/EtOAc) to provide a mixture (1:1) of the two diastereomers of 4-[(3,5-bis-trifluoromethylphenyl)-(2H-tetrazol-5-yl)-methyl]-2-ethyl-3,4-dihydro-2H-quinoxaline-1-carboxylic acid ethyl ester (9) as light yellow film (54.5 mg, 0.103 mmol, 89%). ¹H NMR (CDCl₃) δ 0.76 (m, 3H), 1.16-1.30 (m, 6H), 2.88-3.08 (m, 1.5H), 3.36 (br m, 0.5H), 4.02-4.48 (m, 3H), 6.40-6.80 (m, 4H), 7.41 (d, 1H), 7.71 (s, 1H), 7.95 (d, 2H).

Step E: 4-[(3,5-Bis-trifluoromethylphenyl)-(2-methyl-2H-tetrazol-5-yl)-methyl]-2-ethyl-3,4-dihydro-2H-quinoxaline-1-carboxylic acid ethyl ester (10): To a solution of 4-[(3,5-bis-trifluoromethylphenyl)-(2H-tetrazol-5-yl)-methyl]-2-ethyl-3,4-dihydro-2H-quinoxaline-1-carboxylic acid ethyl ester (10.2 mg, 0.0193 mmol) in acetone (7 mL) was added K₂CO₃ (13.3 mg, 0.0965 mmol). The flask was cooled to 0° C. under a nitrogen atmosphere and MeI (5.5 mg, 0.038 mmol) was added. After 5 minutes of stirring, the reaction was heated to reflux for 1 hour. The reaction was cooled to room temperature, filtered through a sintered glass funnel, and concentrated. The resulting oil was purified by flash chromatography (100% CH₂Cl₂) to provide a mixture (1:1) of the two diastereomers of 4-[(3,5-bis-trifluoromethylphenyl)-(2-methyl-2H-tetrazol-5-yl)-methyl]-2-ethyl-3,4-dihydro-2H-quinoxaline-1-carboxylic acid ethyl ester (10) as a white foam (10.5 mg, 0.00978 mmol, 51%). ¹H NMR (CDCl₃) δ 0.80 (m, 3H), 1.20-1.35 (m, 6H), 3.04 (d, 0.5H), 3.30 (m, 1H), 3.44 (d, 0.5H), 4.15-4.37 (m, 2H), 4.40 (s, 3H), 4.48 (m, 1H), 6.63-7.05 (m, 4H), 7.77 (s, 2H), 7.87 (s, 1H).

### Example 3

### Synthesis of 4-[(3,5-Bis-trifluoromethylphenyl)-methoxycarbonylmethyl]-6,7-dichloro-2-ethyl-3,4-dihydro-2H-quinoxaline-1-carboxylic acid ethyl ester (12)

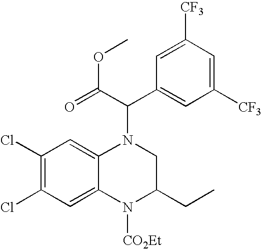

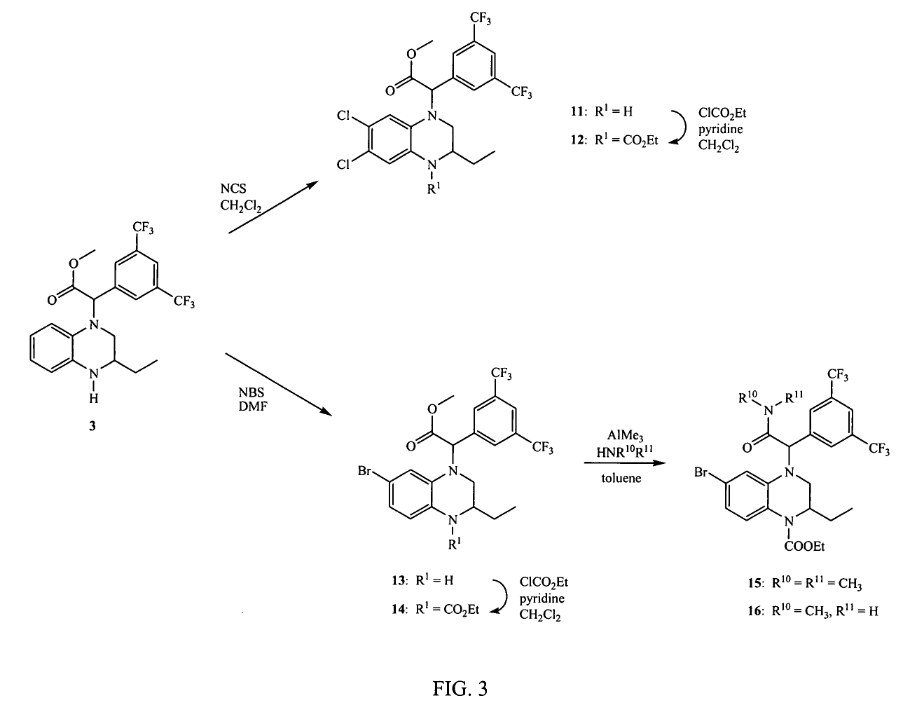

The synthesis of compound (12) according to Example 3 is illustrated in FIG. 3.

Step A: (3,5-Bis-trifluoromethylphenyl)-(6,7-dichloro-3-ethyl-3,4-dihydro-2H-quinoxalin-1-yl)-acetic acid methyl ester (11): To a solution of (3,5-bis-trifluoromethylphenyl)-(3-ethyl-3,4-dihydro-2H-quinoxalin-1-yl)-acetic acid methyl ester (30) prepared according to Example 1 (120 mg, 0.269 mmol) in CH₂Cl₂ (2 mL) under a nitrogen atmosphere was added NCS (35.9 mg, 0.269 mmol). The reaction was stirred at room temperature for 35 minutes. The reaction was diluted with water (30 mL) and extracted twice with EtOAc (2×20 mL). The combined organics were washed twice with brine (30 mL), dried over Na₂SO₄, and concentrated. The resulting oil was purified by flash chromatography (3:1 CH₂Cl₂/hexanes, then 2:1 CH₂C₂/hexanes, then with 100% CH₂Cl₂) to provide (3,5-bis-trifluoromethylphenyl)-(6,7-dichloro-3-ethyl-3,4-dihydro-2H-quinoxalin-1-yl)-acetic acid methyl ester (11) as a yellow film (5.2 mg, 0.011 mmol, 4%).

Step B: 4-[(3,5-Bis-trifluoromethylphenyl)-methoxycarbonylmethyl]-6,7-dichloro-2-ethyl-3,4-dihydro-2H-quinoxaline-1-carboxylic acid ethyl ester (12): To a solution of (3,5-bis-trifluoromethylphenyl)-(6,7-dichloro-3-ethyl-3,4-dihydro-2H-quinoxalin-1-yl)-acetic acid methyl ester (5.2 mg, 0.011 mmol) in CH₂Cl₂ (5 mL) was added pyridine (1.2 mg, 0.015 mmol) and ethyl chloroformate (1.6 mg, 0.015 mmol). The reaction was stirred at room temperature for 1 hour. The reaction was concentrated to dryness. The resulting oil was purified by flash chromatography (100% CH₂Cl₂) to provide two diastereomers of 4-[(3,5-bis-trifluoromethylphenyl)-methoxycarbonylmethyl]-6,7-dichloro-2-ethyl-3,4-dihydro-2H-quinoxaline-1-carboxylic acid ethyl ester (12) as yellow films. (Higher R_{f} by TLC, 2.6 mg, 22%) and (Lower R_{f} by TLC, 2.6 mg, 22%). ¹H NMR (CDCl₃) (Higher R_{f} by TLC) δ 0.87 (t, 3H), 1.28 (t, 3H), 1.44 (m, 2H), 3.05 (dd, 1H), 3.26 (d, 1H), 3.87 (s, 3H), 4.28 (m, 2H), 4.49 (m, 1H), 5.66 (s, 1H), 6.63 (d, 1H), 6.78 (dd, 1H), 7.83 (s, 2H), 7.90 (s, 1H). ¹H NMR (Lower R_{f} by TLC) δ 0.70 (t, 3H), 1.30 (m, 1H), 1.30 (t, 3H), 1.42 (m, 1H), 2.81 (d, 1H), 3.42 (dd, 1H), 386 (s, 3H), 4.23 (m, 2H), 4.42 (m, 1H), 5.75 (s, 1H), 6.76 (s, 1H), 6.79 (d, 1H), 7.75 (s, 2H), 7.91 (s, 1H).

### Example 4

### Synthesis of 4-[(3,5-Bis-trifluoromethylphenyl)-dimethylcarbamoylmethyl]-6-bromo-2-ethyl-3,4-dihydro-2H-quinoxaline-1-carboxylic acid ethyl ester (15)

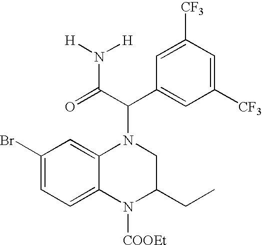

The synthesis of compound (15) according to Example 4 is illustrated in FIG. 3.

Step A: (3,5-Bis-trifluoromethylphenyl)-(7-bromo-3-ethyl-3,4-dihydro-2H-quinoxalin-1-yl)-acetic acid methyl ester (13): To a solution of (3,5-bis-trifluoromethylphenyl)-(3-ethyl-3,4-dihydro-2H-quinoxalin-1-yl)-acetic acid methyl ester (3) prepared according to Example 1 (0.707 g, 1.58 mmol) in DMF (10 mL) under a nitrogen atmosphere was added NBS (846 mg, 4.74 mmol) in 3 portions over 1 hour. The reaction was diluted with saturated Na₂S₂O₃ and extracted twice with CH₂Cl₂ (20 mL). The combined organics were washed twice with brine (30 mL), dried over Na₂SO₄, and concentrated. The resulting oil was purified by column chromatography (Biotage 12m, 1:1 CH₂Cl₂/hexanes, then 3:1 CH₂Cl₂/hexanes) to provide (3,5-bis-trifluoromethylphenyl)-(7-bromo-3-ethyl-3,4-dihydro-2H-quinoxalin-1-yl)-acetic acid methyl ester (13) as a yellow oil (183 mg, 0.349 mmol, 22%).

Step B: 4-[(3,5-Bis-trifluoromethylphenyl)-methoxycarbonylmethyl]-6-bromo-2-ethyl-3,4-dihydro-2H-quinoxaline-1-carboxylic acid ethyl ester (14): To a solution of (3,5-bis-trifluoromethylphenyl)-(7-bromo-3-ethyl-3,4-dihydro-2H-quinoxalin-1-yl)-acetic acid methyl ester (12.0 mg, 0.023 mmol) in CH₂Cl₂ (2 mL) was added pyridine (2.7 mg, 0.034 mmol) followed by ethyl chloroformate (3.7 mg, 0.034 mmol). The reaction was stirred at room temperature for 5 minutes. The reaction was diluted with CH₂Cl₂ (20 mL) and washed with 1N HCl (10 mL), saturated NaHCO₃ (10 mL), dried over Na₂SO₄, and concentrated. The resulting oil was purified by flash chromatography (1:1 CH₂C₂/hexanes, then 4:1 CH₂Cl₂/hexanes) to provide a mixture (1:1) of the two diastereomers of 4-[(3,5-bis-trifluoromethylphenyl)-methoxycarbonylmethyl]-6-bromo-2-ethyl-3,4-dihydro-2H-quinoxaline-1-carboxylic acid ethyl ester (14) as a yellow film (8.5 mg, 63%). ¹H NMR (CDCl₃) (Lower R_{f} by TLC) δ 0.72 (t, 3H), 1.15-1.54 (m, 5H), 2.91 (d, 1H), 3.42 (d, 1H), 3.88 (s, 3H), 4.22 (m, 2H), 4.43 (m, 1H), 5.92 (s, 1H), 6.90 (d, 1H), 6.99 (s, 1H), 7.65 (br d, 1H), 7.88 (s, 2H), 8.01 (s, 1H).

Step C: 4-[(3,5-Bis-trifluoromethylphenyl)-dimethylcarbamoylmethyl]-6-bromo-2-ethyl-3,4-dihydro-2H-quinoxaline-1-carboxylic acid ethyl ester (15): Under a nitrogen atmosphere, AlMe₃ (0.075 mL, 0.15 mmol, 2.0 M in hexanes) was added dropwise to dimethylamine (6.7 mg, 0.15 mmol, dissolved in 0.3 mL of toluene). This mixture was stirred for 5 minutes at room temperature, then added to a solution of 4-[(3,5-bis-trifluoromethylphenyl)-methoxycarbonylmethyl]-6-bromo-2-ethyl-3,4-dihydro-2H-quinoxaline-1-carboxylic acid ethyl ester (8.9 mg, 0.015 mmol) in toluene (3 mL) under a nitrogen atmosphere. The reaction was heated to 95° C. for 14 hours. The reaction was cooled to room temperature and diluted with 10% Rochelle's salt solution (10 mL). The aqueous layer was extracted with EtOAc (20 mL). The organic layer was dried over Na₂SO₄, and concentrated. The resulting oil was purified by flash chromatography (100% CH₂Cl₂, then 2:1 CH₂Cl₂/EtOAc) to provide a mixture (1:1) of the two diastereomers of 4-[(3,5-bis-trifluoromethylphenyl)-dimethylcarbamoylmethyl]-6-bromo-2-ethyl-3,4-dihydro-2H-quinoxaline-1-carboxylic acid ethyl ester (15) as a yellow solid (3.2 mg, 35% yield). ¹H NMR (CDCl₃) (Lower R_{f} by TLC) δ 0.83 (t, 3H), 1.22-1.55 (m, 6H), 2.95 (s, 3H), 3.08 (s, 3H), 3.17 (d, 1H), 3.41 (dd, 1H), 4.23 (m, 2H), 4.52 (m, 1H), 5.68 (s, 1H), 6.58 (s, 1H), 6.89 (dd, 1H), 7.71 (s, 2H), 7.88 (s, 1H).

### Example 5

### Synthesis of 4-[(3,5-Bis-trifluoromethylphenyl)-methylcarbamoylmethyl]-6-bromo-2-ethyl-3,4-dihydro-2H-quinoxaline-1-carboxylic acid ethyl ester (16)

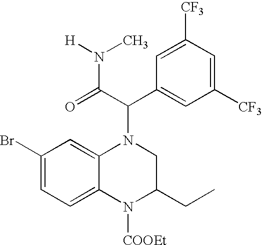

The synthesis of compound (16) according to Example 5 is illustrated in FIG. 3.

Under a nitrogen atmosphere, AlMe₃ (0.167 mL, 0.335 mmol, 2.0 M in hexanes) was added dropwise to methylamine (10.4 mg, 0.335 mmol, dissolved in 0.3 mL of toluene). This mixture was stirred for 5 minutes at room temperature, then added to a solution of 4-[(3,5-bis-trifluoromethylphenyl)-methoxycarbonyl-methyl]-6-bromo-2-ethyl-3,4-dihydro-2H-quinoxaline-1-carboxylic acid ethyl ester (14) prepared according to Example 4 (40.0 mg, 0.067 mmol) in toluene (3 mL) under a nitrogen atmosphere. The reaction was heated to 80° C. for 40 minutes. The reaction was cooled to room temperature and diluted with 10% Rochelle's salt solution (10 mL). The aqueous layer was extracted with EtOAc (20 mL). The organic layer was dried over Na₂SO₄, and concentrated. The resulting oil was purified by flash chromatography (100% CH₂Cl₂, then 9:1 CH₂Cl₂₁EtOAc) to provide the two diastereomers of 4-[(3,5-bis-trifluoromethylphenyl)-methylcarbamoyl-methyl]-6-bromo-2-ethyl-3,4-dihydro-2H-quinoxaline-1-carboxylic acid ethyl ester (16) as colorless oils. (Higher R_{f} by TLC, 5.7 mg, 14%) and (Lower R_{f} by TLC, 6.2 mg, 16%). ¹H NMR (CDCl₃) (Higher R_{f} by TLC) δ 0.93 (t, 3H), 1.22-1.53 (m, 5H), 2.97 (d, 3H), 3.08 (d, 1H), 4.21 (m, 2H), 4.52 (m, 1H), 5.50 (s, 1H), 6.32 (m, 1H), 6.83 (s, 1H), 6.96 (dd, 1H), 7.73 (br d, 1H), 7.73 (s, 2H), 7.88 (s, 1H). ¹H NMR (Lower R_{f} by TLC) δ 0.73 (t, 3H), 1.13 (m, 1H), 1.24-1.44 (m, 5H), 2.95 (d, 3H), 3.38 (dd, 1H), 4.25 (m, 2H), 4.44 (br s, 1H), 5.50 (s, 1H), 6.02 (m, 1H), 6.82 (s, 1H), 6.94 (dd, 1H), 7.51 (br d, 1H), 7.75 (s, 2H), 7.88 (s, 1H).

### Example 6

### Synthesis of 4-[1-(3,5-Bis-trifluoromethylphenyl)-2-oxo-propyl]-2-ethyl-6-trifluoromethyl-3,4-dihydro-2H-quinoxaline-1-carboxylic acid ethyl ester (22)

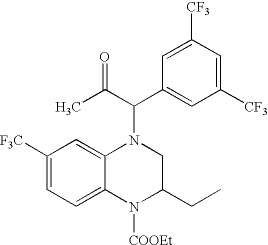

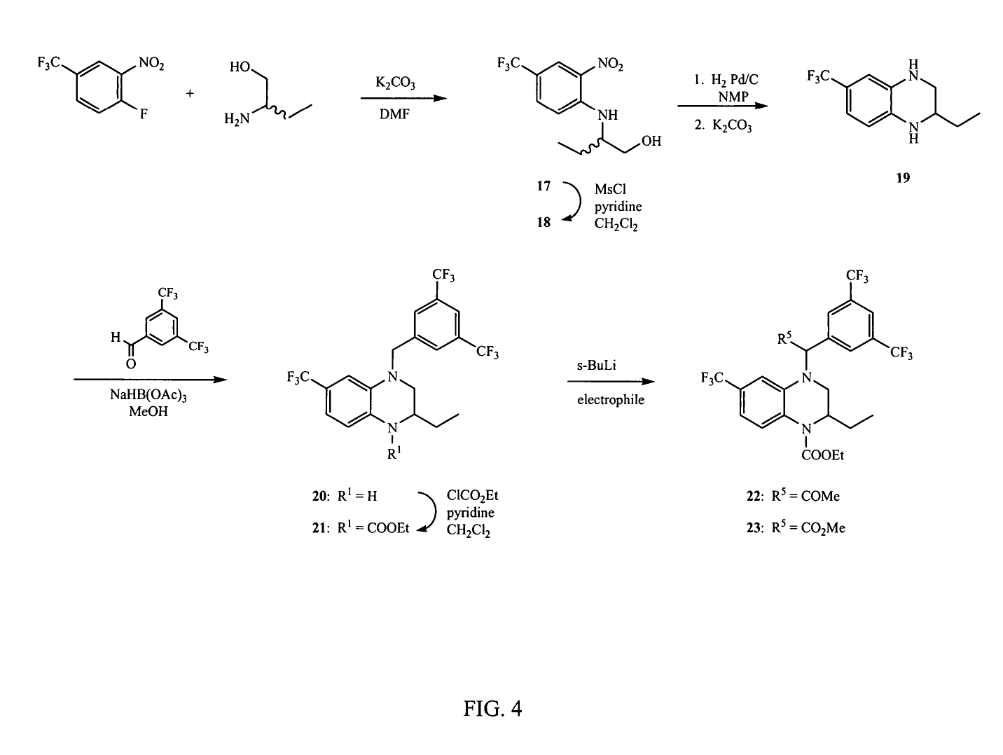

The synthesis of compound (22) according to Example 6 is illustrated in FIG. 4.

Step A: 2-(2-Nitro-4-trifluoromethylphenylamino)-butan-1-ol (17): To a solution of 4-fluoro-3-nitrobenzotrifluoride (11.80 g, 56.43 mmol) in DMF (175 mL) under a nitrogen atmosphere was added K₂CO₃ (7.80 g, 56.43 mmol) and (+/−) 2-amino-1-butanol. The reaction mixture was heated to 80° C. for 18 hours and cooled to room temperature. The solids were filtered and washed with Et₂O (500 mL). The combined organics were washed with water (5×300 mL), brine (300 mL), dried over MgSO₄, passed through a silica gel plug (EtOAc elution) and concentrated to yield 2-(2-nitro-4-trifluoromethylphenylamino)-butan-1-ol (17) as a bright yellow solid (15.37 g, 55.24 mmol, 98%).

Step B: Methanesulfonic acid 2-(2-nitro-4-trifluoromethylphenylamino)-butyl ester (18): To a solution of 2-(2-nitro-4-trifluoromethylphenylamino)-butan-1-ol (12.0 g, 43.1 mmol) in CH₂Cl₂ (20 mL) under a nitrogen atmosphere was added pyridine (5.12 g, 64.7 mmol) followed by MsCl (5.19 g, 45.3 mmol). The reaction was stirred at room temperature for 3 hours. The yellow slurry was diluted with CH₂Cl₂ (250 mL) and washed with 1N HCl (150 mL), saturated NaHCO₃ (150 mL), brine (150 mL), dried over Na₂SO₄, filtered to yield methanesulfonic acid 2-(2-nitro-4-trifluoromethylphenylamino)-butyl ester (18) as a yellow oil (15.0 g, 42.1 mmol, 98%), which was used without further purification.

Step C: 2-Ethyl-6-trifluoromethyl-1,2,3,4-tetrahydroquinoxaline (19): Methanesulfonic acid 2-(2-nitro-4-trifluoromethylphenylamino)-butyl ester (9.04 g, 25.4 mmol) was dissolved in NMP (150 mL) and hydrogenated at 40 psi over 10% Pd/C (degussa, 1.0 g) for 4 days. The reaction mixture was filtered through celite, K₂CO₃ (10.5 g, 76.1 mmol) was added, followed by tetrabutylammonium iodide (catalytic), and heated to 100° C. for 18 hours. The reaction mixture was cooled to room temperature. The solids were removed by filtration and washed with EtOAc (500 mL). The combined organics were washed with brine (3×300 mL), dried over MgSO₄, filtered and concentrated to yield a yellow oil. The crude material was purified by flash chromatography (100% hexanes to 10% EtOAc) to yield 2-ethyl-6-trifluoromethyl-1,2,3,4-tetrahydroquinoxaline (19) as a yellow solid (2.0 g, 34%).

Step D: 1-(3,5-Bis-trifluoromethylbenzyl)-3-ethyl-7-trifluoromethyl-1,2,3,4-tetrahydroguinoxaline (20): To a solution of 2-ethyl-6-trifluoromethyl-1,2,3,4-tetrahydroquinoxaline (2.00 g, 8.69 mmol) in DCE (100 mL) under a nitrogen atmosphere was added molecular sieves (3 Å), 3,5-bis(trifluoromethyl)benzaldehyde (2.10 g, 8.69 mmol) and acetic acid (0.13 g, 2.17 mmol). The mixture was stirred at room temperature for 1.5 hours and then NaHB(OAc)₃ (2.21 g, 10.4 mmol) was added in small portions every 15 minutes over 3 hours. The reaction was stirred for 6 hours and judged to be approximately 50% complete by LC-MS. Additional 3,5-bis(trifluoromethyl)benzaldehyde (2.10 g, 8.69 mmol), NaHB(OAc)₃ (2.21 g, 10.4 mmol) and molecular sieves were added and the reaction mixture was stirred for 16 hours at which point it was judged to be complete by LC-MS. The reaction mixture was filtered, diluted with EtOAc (400 mL), washed with saturated NaHCO₃ (100 mL), brine (100 mL), dried over Na₂SO₄, and filtered to yield dark yellow oil. The crude product was purified by flash chromatography (100% hexanes to 5% EtOAc) to yield 1-(3,5-bis-trifluoromethylbenzyl)-3-ethyl-7-trifluoromethyl-1,2,3,4-tetrahydroquinoxaline (20) as a yellow oil (1.76 g, 3.86 mmol, 44%).

Step E: 4-(3,5-Bis-trifluoromethyl-benzyl)-2-ethyl-6-trifluoromethyl-3,4-dihydro-2H-quinoxaline-1-carboxylic acid ethyl ester (21): To a solution of 1-(3,5-bis-trifluoromethylbenzyl)-3-ethyl-7-trifluoromethyl-1,2,3,4-tetrahydroquinoxaline (1.76 g, 3.86 mmol) in CH₂Cl₂ (50 mL) under a nitrogen atmosphere at 0° C. was added pyridine (0.91 g, 11.57 mmol) and ethyl chloroformate (0.84 g, 7.71 mmol) over 30 minutes. The reaction mixture was stirred at 0° C. for 2 hours at which point it was judged to be complete by LC-MS. The reaction was diluted with EtOAc (200 mL), washed with half saturated NH₄Cl (2×100 mL), saturated NaHCO₃ (100 mL), brine (100 mL), dried over Na₂SO₄, filtered and concentrated. The crude yellow oil was purified by flash chromatography (100% hexane to 10% EtOAc) to yield 4-(3,5-bis-trifluoromethylbenzyl)-2-ethyl-6-trifluoromethyl-3,4-dihydro-2H-quinoxaline-1-carboxylic acid ethyl ester (21) as a pale yellow oil (0.55 g, 1.04 mmol, 27%).

Step F: 4-[i-(3,5-Bis-trifluoromethylphenyl)-2-oxo-propyl]-2-ethyl-6-trifluoromethyl-3,4-dihydro-2H-quinoxaline-1-carboxylic acid ethyl ester (22): To a flame-dried flask under nitrogen was added 4-(3,5-bis-trifluoromethylbenzyl)-2-ethyl-6-trifluoromethyl-3,4-dihydro-2H-quinoxaline-1-carboxylic acid ethyl ester (0.0540 g, 0.102 mmol), THF (1.5 mL) and HMPA (0.5 mL). The resulting solution was cooled to −78° C. and s-BuLi (0.234 mL, 0.281 mmol, 1.20 M in cyclohexane) was added slowly over 5 minutes. The purple reaction mixture was stirred at −78° C. for 1 hour during which time it turned green. The reaction was cooled to −78° C. and acetyl chloride (0.0401 g, 0.511 mmol) was added dropwise over 5 minutes. The reaction was stirred at −78° C. for 1.5 hours, 0° C. for 4 hours and room temperature for 16 hours. The reaction mixture was quenched with water (1 mL), diluted with EtOAc (40 mL), washed with water (3×40 mL), brine (2×40 mL), dried over MgSO₄, filtered and concentrated. The crude product was purified by preparative TLC (20% EtOAc/hexanes) and further purified by a second preparative TLC (100% DCM) to yield 4-[1-(3,5-bis-trifluoromethylphenyl)-2-oxo-propyl]-2-ethyl-6-trifluoromethyl-3,4-dihydro-2H-quinoxaline-1-carboxylic acid ethyl ester (22) as a yellow oil (0.0013 g, 0.0023 mmol, 2.2% yield). ¹H NMR (CDCl₃) δ 0.75 (t, 3H), 1.13-1.22 (m, 1H), 1.32 (t, 3H), 1.38-1.46 (m, 1H), 2.04 (s, 3H), 2.98 (d, 1H), 3.35 (dd, 1H), 4.25 (q, 2H), 4.45-4.50 (m, 1H), 5.85 (s, 1H), 7.03 (d, 1H), 7.17 (s, 1H), 7.71-7.78 (m, 1H), 7.83 (s, 2H), 7.90 (s, 1H).

### Example 7

### Synthesis of 4-[(3,5-Bis-trifluoromethylphenyl)-methoxycarbonyl-methyl]-2-ethyl-6-trifluoromethyl-3,4-dihydro-2H-quinoxaline-1-carboxylic acid ethyl ester (23)

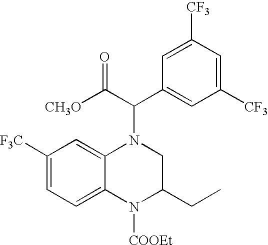

The synthesis of compound 23 according to Example 7 is illustrated in FIG. 4.

A solution of s-BuLi (0.23 mL, 0.281 mmol, 1.20 M in cyclohexane) was slowly added to a solution of 4-(3,5-bis-trifluoromethylbenzyl)-2-ethyl-6-trifluoromethyl-3,4-dihydro-2H-quinoxaline-1-carboxylic acid ethyl ester (21); prepared as described in Example 6 (54 mg, 0.102 mmol) in THF/HMPA (2:1, 1.5 mL) at −78° C. under a nitrogen atmosphere. After 45 minutes, methyl chloroformate (50 mg, 0.500 mmol) was added and the reaction was held at −78° C. After 4 hours, the reaction was warmed to 0° C. for 2 hours, and then stored at 7° C. After 14 hours the reaction mixture was partitioned between saturated NaHCO₃ and EtOAc (1:1, 40 mL). The aqueous layer was removed and the organic layer was further diluted with hexane (10 mL). The organic layer was washed with brine (4×50 mL), dried (MgSO₄), and concentrated. The concentrate was purified via preparative TLC (0.5 mm plate, 100% CH₂Cl₂) to give 4-[(3,5-bis-trifluoromethylphenyl)-methoxycarbonylmethyl]-2-ethyl-6-trifluoromethyl-3,4-dihydro-2H-quinoxaline-1-carboxylic acid ethyl ester (23) (6 mg, 0.010 mmol, 10% as a 1.4:1 mixture of diastereomers). ¹H NMR (CDCl₃) (higher R_{f} diastereomer): δ 7.93 (s, 1H), 7.82 (d, 1H), 7.77 (s, 2H), 7.08 (d, 1H), 6.99 (s, 1H), 5.82 (s, 1H), 4.46 (s, 1H), 4.30-4.21 (m, 2H), 3.86 (s, 3H), 3.45 (dd, 1H), 2.83 (d, 1H), 1.47-1.38 (m, 2H), 1.31 (t, 3H), 0.70 (t, 3H). (lower R_{f} diastereomer): δ 7.91 (s, 1H), 7.75 (s, 2H), 7.64 (d, 1H), 7.05 (d, 1H), 6.84 (s, 1H), 5.69 (s, 1H), 4.53 (s, 1H), 4.31-4.17 (m, 2H), 3.86 (s, 3H), 3.30 (d, 1H), 3.13 (dd, 1H), 1.49-1.40 (m, 2H), 1.30 (t, 3H), 0.86 (t, 3H).

### Example 8

### Synthesis of 4-[(3,5-bis-trifluoromethylphenyl)-(2-methyl-2H-tetrazol-5-yl)-methyl]-2-ethyl-6-trifluoromethyl-3,4-dihydro-2H-quinoxaline-1-carboxylic acid ethyl ester (27)

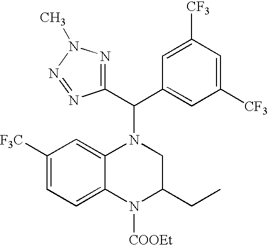

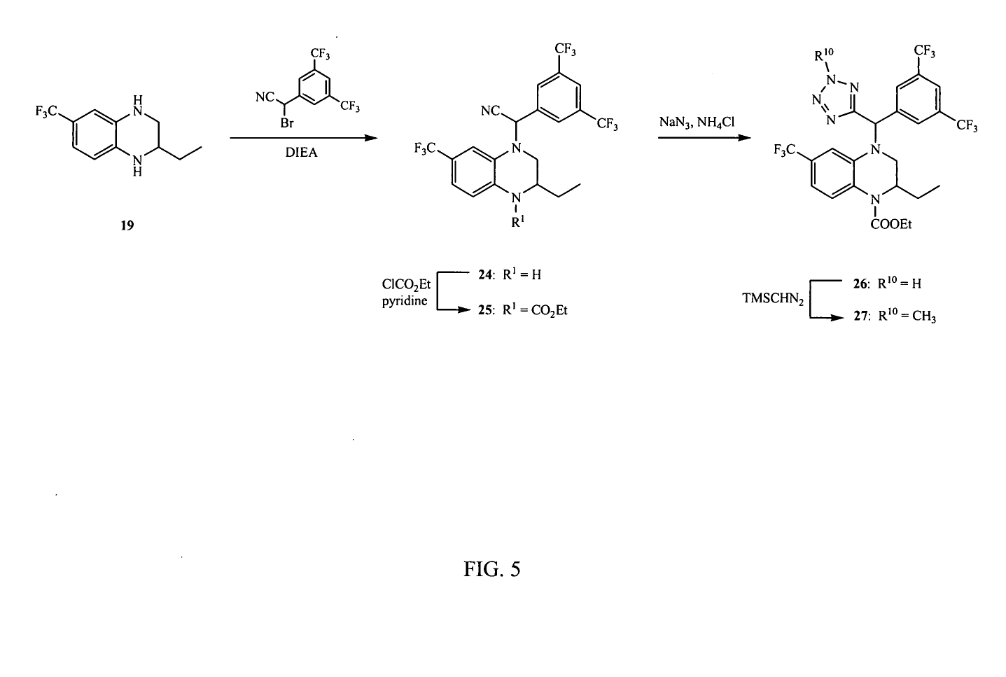

The synthesis of compound (27) according to Example 8 is illustrated in FIG. 5.

Step A: (3,5-Bis-trifluoromethylphenyl)-(3-ethyl-7-trifluoromethyl-3,4-dihydro-2H-quinoxalin-1-yl)-acetonitrile (24): To a solution of 2-ethyl-6-trifluoromethyl-1,2,3,4-tetrahydroquinoxaline (389 mg, 1.69 mmol) and DIEA (440 mg, 3.40 mmol) in DMF under N₂ was added (3,5-bistrifluoromethylphenyl)-bromoacetonitrile (670 mg, 2.0 mmol) dropwise. After 1 hour additional (3,5-bis-trifluoromethylphenyl)-bromoacetonitrile (670 mg, 2.0 mmol) was added. After stirring for 14 hours additional (3,5-bis-trifluoromethylphenyl)-bromoacetonitrile (670 mg, 2.0 mmol) was added and the mixture then stirred for 2 hours. The reaction mixture was partitioned between EtOAc (100 mL) and water (100 mL). The aqueous layer was removed and the organic layer was diluted with hexanes (50 mL) and washed with brine (3×150 mL). The organic layer was dried (MgSO₄) and concentrated. The concentrate was purified via flash chromatography (100% hexanes to 5% EtOAc/hexanes) to yield 579 mg (57%) of (3,5-bis-trifluoromethylphenyl)-(3-ethyl-7-trifluoromethyl-3,4-dihydro-2H-quinoxalin-1-yl)-acetonitrile (24).

Step B: 4-[(3,5-Bis-trifluoromethylphenyl)-cyanomethyl]-2-ethyl-6-trifluoromethyl-3,4-dihydro-2H-quinoxaline-1-carboxylic acid ethyl ester (25): To a solution of (3,5-bis-trifluoromethylphenyl)-(3-ethyl-7-trifluoromethyl-3,4-dihydro-2H-quinoxalin-1-yl)-acetonitrile (245 mg, 0.509 mmol) in CH₂Cl₂ (20 mL) with pyridine (2 mL) as co-solvent was added ethyl chloroformate (170 mg, 1.50 mmol). After 1 hour, additional ethyl chloroformate (170 mg, 1.5 mmol) was added. After an additional hour the mixture was partitioned between saturated NaHCO₃ (100 mL) and CH₂Cl₂ (100 mL). The organic layer was removed, dried (MgSO₄) and concentrated. The concentrate was purified via flash chromatography (100% hexanes to 5% EtOAc/hexanes) to yield 200 mg (71%) of 4-[(3,5-bis-trifluoromethylphenyl)-cyanomethyl]-2-ethyl-6-trifluoromethyl-3,4-dihydro-2H-quinoxaline-1-carboxylic acid ethyl ester (25).

Step C: 4-[(3,5-Bis-trifluoromethylphenyl)-(2H-tetrazol-5-yl)-methyl]-2-ethyl-6-trifluoromethyl-3,4-dihydro-2H-quinoxaline-1-carboxylic acid ethyl ester (26): To a solution of 4-[(3,5-bis-trifluoromethylphenyl)-cyanomethyl]-2-ethyl-6-trifluoromethyl-3,4-dihydro-2H-quinoxaline-1-carboxylic acid ethyl ester (200 mg, 0.361 mmol) in DMF (20 mL) was added NH₄Cl (97 mg, 1.8 mmol) and NaN₃ (120 mg, 1.8 mmol). The mixture was heated to 50° C. for 6 hours. Upon cooling the reaction mixture was partitioned between EtOAc (100 mL) and saturated NH₄Cl (100 mL). The aqueous layer was removed and the organic was washed with brine (2×100 mL). The organic layer was dried (MgSO₄) and concentrated. The concentrate was purified via flash chromatography (100% hexanes to 100% EtOAc) to yield 30 mg (14%) of 4-[(3,5-bis-trifluoromethylphenyl)-(2H-tetrazol-5-yl)-methyl]-2-ethyl-6-trifluoromethyl-3,4-dihydro-2H-quinoxaline-1-carboxylic acid ethyl ester (26).

Step D: 4-[(3,5-Bis-trifluoromethylphenyl)-(2-methyl-2H-tetrazol-5-yl)-methyl]-2-ethyl-6-trifluoromethyl-3,4-dihydro-2H-quinoxaline-1-carboxylic acid ethyl ester (27): To a solution of 4-[(3,5-bis-trifluoromethylphenyl)-(2H-tetrazol-5-yl)-methyl]-2-ethyl-6-trifluoromethyl-3,4-dihydro-2H-quinoxaline-1-carboxylic acid ethyl ester (8 mg, 0.013 mmol) in THF/MeOH (5:1, 4 mL) was added a solution of TMSCHN₂ (0.020 mL, 2.0 M in hexanes). The reaction mixture was concentrated and the crude mixture was purified via preparative TLC (0.5 mm, 100% CH₂Cl₂) to yield 4.5 mg (55%) of 4-[(3,5-bis-trifluoromethylphenyl)-(2-methyl-2H-tetrazol-5-yl)-methyl]-2-ethyl-6-trifluoromethyl-3,4-dihydro-2H-quinoxaline-1-carboxylic acid ethyl ester (27) as an approximately 2:1 mixture of diastereomers. ¹H NMR (CDCl₃) δ 7.94-7.88 (m, 1.5H), 7.80-7.74 (m, 3.5H), 7.63-7.61 (m, 0.5H) 7.19 (s, 1H), 7.10-7.01 (m, 2.5H), 6.75 (s, 1H), 6.65 (s, 1H), 4.53-4.46 (m, 1.5H), 4.41 (s, 4.5H), 4.29-4.18 (m, 3H), 3.52 (d, 0.5H), 3.35 (dd, 1H), 3.29 (dd, 0.5H), 3.07 (d, 1H), 1.33-1.22 (m, 7H), 0.83-0.76 (m, 4.5H).

### Example 9

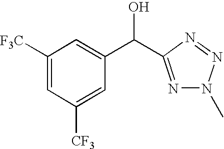

### Synthesis of (3,5-bis(trifluoromethyl)phenyl)(2-methyl-2H-tetrazol-5-yl)methanol (30)

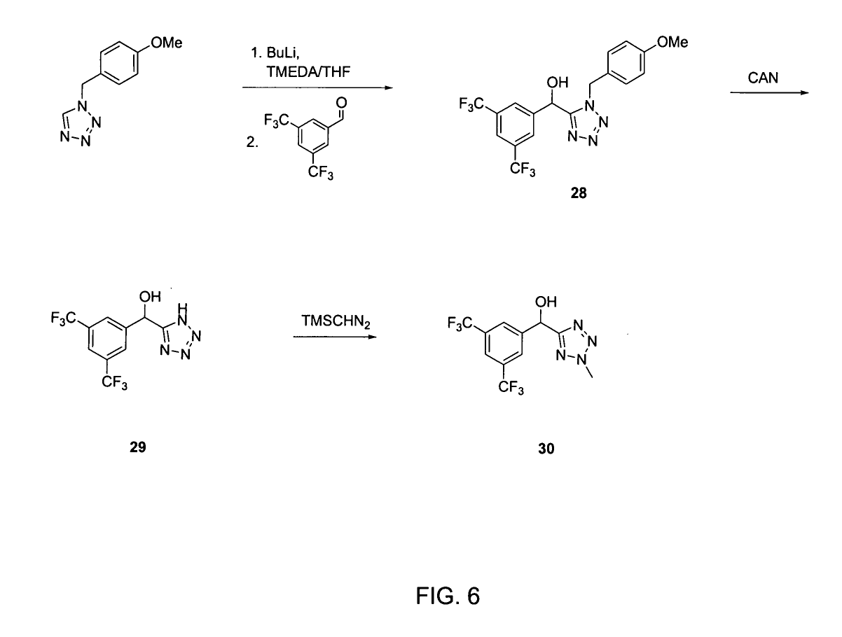

The synthetic scheme for the preparation of compound (30) according to Example 9 is shown in FIG. 6.

Step A: (3,5-bis(trifluoromethyl)phenyl)(1-(4-methoxybenzyl)-1H-tetrazol-5-yl)methanol (28): A solution of of BuLi (0.49 mL, 2.33 M) was added to 1-(4-methoxybenzyl)-1H-tetrazole (Tet. Lett. 1995, 36, 1759-1762) in THF/TMEDA (10:1, 11 mL) at −88° C. (liquid N₂, MeOH). After ˜10 minutes the 3,5-bis(trifluoromethyl)benzaldehyde was added and the mixture was stirred at −78 C for ˜30 minutes. The mixture was removed from the ice bath and allowed to warm to room temperature and stir for 90 minutes. The reaction was quenched with the addition of sat. NH₄Cl (5 mL). The mixture was partitioned between EtOAc (50 mL) and brine (50 mL). The phases were separated and the aqueous phase was extracted with EtOAc (2×50 mL), dried (MgSO₄), filtered and concentrated. The concentrate was purified via flash chromatography (100% hexanes to 10% EtOAc/hexanes) to yield (3,5-bis(trifluoromethyl)phenyl)(1-(4-methoxybenzyl)-1H-tetrazol-5-yl)methanol (28) (186 mg, 38%). ¹H NMR (CDCl₃) δ 7.71 (s, 1H), 7.62 (s, 2H), 6.87 (d, 2H), 2.64 (d, 2H), 6.34 (br, s, 1H), 5.60-5.51 (m, 2H), 5.09 (br s, 1H), 3.72 (s, 3H).

Step B: (3,5-bis(trifluoromethyl)phenyl)(1H-tetrazol-5-yl)methanol (29): To a solution of (3,5-bis(trifluoromethyl)phenyl)(1-(4-methoxybenzyl)-1H-tetrazol-5-yl)methanol (19.4 g, 44.9 mmol) in MeCN/water (3:1, 400 mL) was added ammonium cerium nitrate (98.4 g, 179 mmol). After 1 hour the reaction was judged complete by TLC and HPLC. The mixture was poured into sat. NaHCO₃ (400 mL) and then 1.0 M HCl/brine (1:2, 600 mL) was added and the mixture was extracted with EtOAc (4×300 mL). The combined organics were dried and concentrated. A mixture of DCM/hexane (2:1, 300 mL) was added and (3,5-bis(trifluoromethyl)phenyl)(1H-tetrazol-5-yl)methanol (29) precipitated out of solution (8.3 g, 59%). ¹H NMR (CD₃OD) δ 8.12 (s, 2H), 7.92 (s, 1H), 6.37 (s, 1H).

Step C: (3,5-bis(trifluoromethyl)phenyl)(2-methyl-2H-tetrazol-5-yl)methanol (30): A solution of TMS-diazomethane (15 mL, 2.0 M in hexanes) was added to (3,5-bis(trifluoromethyl)phenyl)(1H-tetrazol-5-yl)methanol (8.30 g, 27 mmol) in THF/MeOH (4:1, 200 mL) at room temperature. After gas evolution had ceased the reaction mixture was concentrated and purified via flash chromatography (100% hexanes to 30% EtOAc) to give (3,5-bis(trifluoromethyl)phenyl)(2-methyl-2H-tetrazol-5-yl)methanol (30) (5.50 g 63%) and 2.62 g of the N1 regioisomer. ¹H NMR (CDCl₃) δ 8.00 (s, 2H), 7.85 (s, 1H), 6.27 (d, 1H), 4.36 (s, 3H), 3.56 (d, 1H).

### Example 10

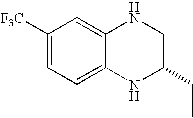

### Synthesis of (S)-2-ethyl-6-(trifluoromethyl)-1,2,3,4-tetrahydroquinoxaline (35)

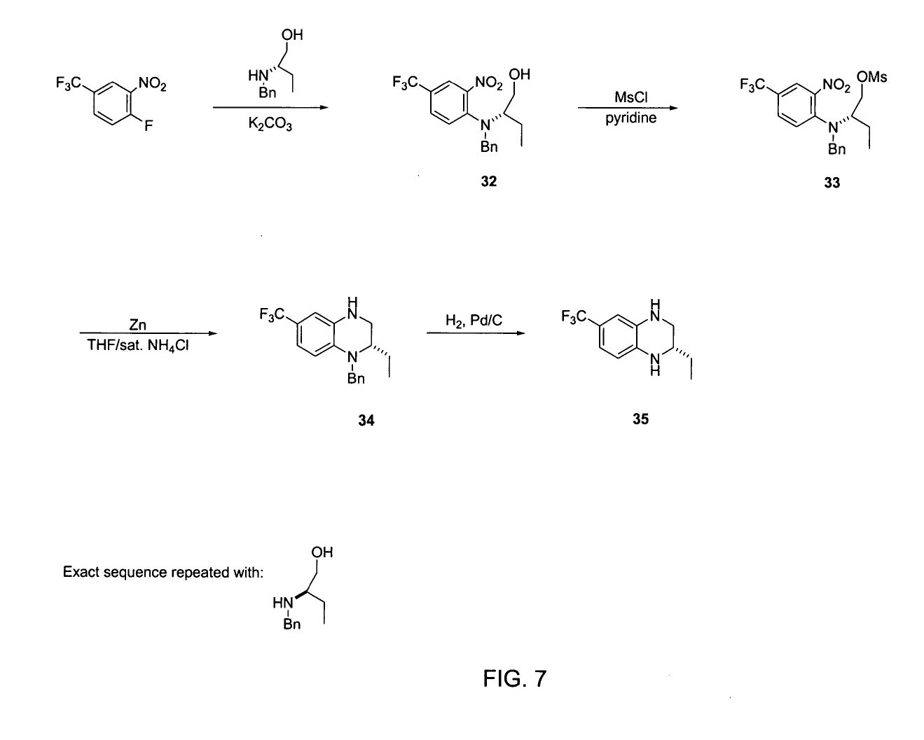

The synthetic scheme for the preparation of compound (35) according to Example 10 is shown in FIG. 7.

Step A: (S)-2-(benzyl(2-nitro-4-(trifluoromethyl)phenyl)amino)butan-1-ol (32): To a solution of (S)-2-(benzylamino)butan-1-ol (4.5 g, 25 mmol, Bioorg. Med. Chem. Lett. 2004, 14, 313-316) and 1-fluoro-2-nitro-4-(trifluoromethyl)benzenefluoride (5.2 g, 25 mmol) in DMF was added K₂CO₃ (3.40 g, 25 mmol) and the mixture was heated to 80° C. for 14 hours. Upon cooling the solid was filtered and the cake was washed with EtOAc (200 mL). The filtrate was concentrated and then purified via flash chromatography (100% hexanes to 20% EtOAc/hexanes) to give (S)-2-(benzyl(2-nitro-4-(trifluoromethyl)phenyl)amino)butan-1-ol (32) (2.48 g, 27%). ¹H NMR (CDCl₃) δ 7.37-7.21 (m, 8H), 4.80 (d, 1H), 4.06 (d, 1H), 3.88-3.77 (m, 1H), 3.61-3.54 (m, 2H), 1.8-1.4 (m, 2H), 0.70 (t, 3H).

Step B: (S)-2-(benzyl(2-nitro-4-(trifluoromethyl)phenyl)amino)butyl methanesulfonate (33): Methanesulfonyl chloride (0.771 g, 6.73 mmol) was added to a solution of (S)-2-(benzyl(2-nitro-4-(trifluoromethyl)phenyl)amino)butan-1-ol (2.48 g, 6.73 mmol) and pyridine (0.533 g, 6.73 mmol) in DCM (50 mL). After 14 hours the mixture was partitioned between DCM and sat. NaHCO3. The organic layer was removed and the aqueous was extracted with DCM (2×100 mL). The combined organics were dried and concentrated. The concentrate was purified via flash chromatography (100% hexanes to 20% EtOAc) to yield (S)-2-(benzyl(2-nitro-4-(trifluoromethyl)phenyl)amino)butyl methanesulfonate (33) (2.27 g, 76%). ¹H NMR (CDCl₃) δ 7.35-7.19 (m, 8H), 4.80 (d, 1H), 4.06 (d, 1H), 3.68 (s, 3H), 3.61-3.51 (m, 2H), 1.80-1.50 (m, 2H), 0.845 (t, 3H).

Step C: (S)-1-benzyl-2-ethyl-6-(trifluoromethyl)-1,2,3,4-tetrahydroquinoxaline (34): To a solution of (S)-2-(benzyl(2-nitro-4-(trifluoromethyl)phenyl)amino)butyl methanesulfonate (2.2 g, 4.9 mmol) in THF (6 mL) and sat. NH₄Cl (5 mL) was added zinc dust (2.6 g, 39 mmol). After 3 hours the reaction was judged complete by LCMS. The excess zinc was filtered and the filtrate was partitioned between sat. NaHCO₃ and EtOAc. The aqueous layer was removed and the organics were washed with brine. The organic layer was dried, filtered and concentrated. The concentrate was purified via flash chromatography (100% hexanes to 10% EtOAc) to give (S)-1-benzyl-2-ethyl-6-(trifluoromethyl)-1,2,3,4-tetrahydroquinoxaline (34) (865 mg, 55%). ¹H NMR (CDCl₃) δ 7.34-7.23 (m, 5H), 6.80 (d, 1H), 6.72 (s, 1H), 6.38 (d, 1H), 4.52 (dd, 2H), 3.41-3.29 (m, 3H), 1.73-1.65 (m, 2H), 0.92 (t, 3H).

Step D: (S)-2-ethyl-6-(trifluoromethyl)-1,2,3,4-tetrahydroquinoxaline (35): To a solution of (S)-1-benzyl-2-ethyl-6-(trifluoromethyl)-1,2,3,4-tetrahydroquinoxaline (270 mg, 0.843 mmol) in MeOH/EtOAc (1:1, 100 mL) was added Pd/C (10 mg, 10% wt, Degussa type). The suspension was placed under a H₂ atmosphere and hydrogenated (50 PSI) utilizing a Parr hydrogenator. After 1 hour the reaction appeared complete by TLC. The mixture was filtered (GF/F) and concentrated to yield (S)-2-ethyl-6-(trifluoromethyl)-1,2,3,4-tetrahydroquinoxaline (35) (190 mg, 99%). ¹H NMR (CDCl₃) δ 6.82 (d, 1H), 6.69 (s, 1H), 6.47 (d, 1H), 3.39 (dd, 1H), 3.35-3.29 (m, 1H), 3.08-3.04 (dd, 1H), 1.58-1.50 (m, 2H), 1.01 (t, 3H).

### Example 11

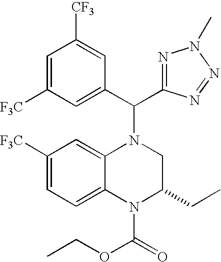

### Synthesis of (2S)-ethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (38-D 1)

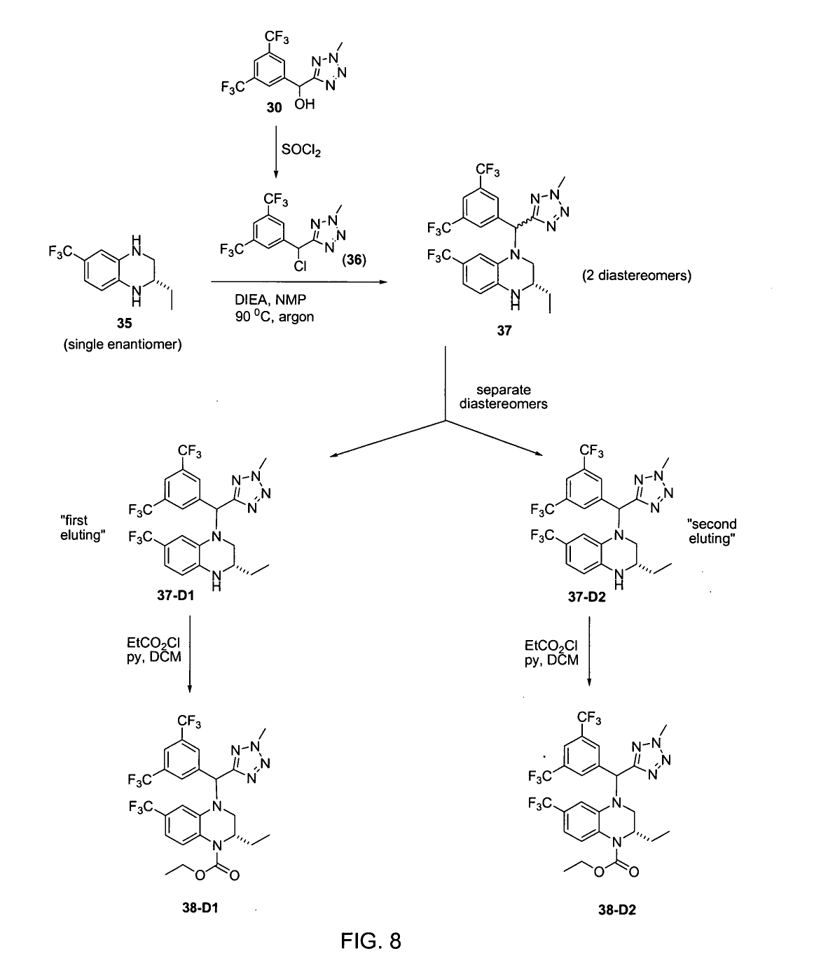

The synthetic scheme for the preparation of compounds (38-D1) according to Example 11 is shown in FIG. 8.

Step A: (3S)-1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline (37-D1) and (37-D2): Thionyl chloride 200 mg, 1.7 mmol) was added to a solution of (3,5-bis(trifluoromethyl)phenyl)(2-methyl-2H-tetrazol-5-yl)methanol (30) (prepared as in Example 9) (550 mg, 1.7 mmol) in DCM (20 mL) under argon. After 18 hours the mixture was concentrated and then rediluted in DMF (3 mL) to provide 5-((3,5-bis(trifluoromethyl)phenyl)chloromethyl)-2-methyl-2H-tetrazole (36). The DMF solution containing the chloro derivative (36) was added to (S)-2-ethyl-6-(trifluoromethyl)-1,2,3,4-tetrahydroquinoxaline (35) (prepared as in Example 10) (190 mg, 0.83 mmol) and DIEA (110 mg, 0.83 mmol) in DMF (3 mL). The mixture was stirred at 90° C. for 14 hours. Upon cooling the vessel contents were partitioned between EtOAc (100 mL) and water (100 mL). The phases were separated and the organic layer was washed with water (3×100 mL). The organic layer was dried (MgSO₄) and concentrated. The concentrate was purified via flash chromatography (100% hexanes to 10% EtOAc) to yield ˜180 mg of alkylated product as a ˜1:1 mixture of diastereomers (37-D1) and (37-D2). A portion (35 mg) of the diastereomer mixture was separated via preparative TLC (˜45% ether/hexanes, 21 mm plates) to give (37-D1) (“first eluting” diastereomer) and (37-D2) (“second eluting” diastereomer). 37-D1: ¹H NMR (CDCl₃) δ 7.88 (s, 1H), 7.82 (s, 2H), 7.04 (s, 1H), 6.92 (d, 1H), 6.61 (s, 1H), 6.50 (d, 1H), 4.41 (s, 3H), 4.30 (br s, 1H), 3.35 (dd, 1H), 3.13-3.11 (m, 1H), 2.96 (dd, 1H), 1.56-1.30 (m, 2H), 0.71 (t, 3H). 37-D2: ¹H NMR (CDCl₃) δ 7.88 (s, 1H), 7.73 (s, 2H), 7.07 (s, 1H), 6.93 (d, 1H), 6.62 (s, 1H), 6.51 (d, 1H), 4.42 (s, 3H), 4.12 (br s, 1H), 3.40-3.30 (m, 1H), 3.23 (dd, 1H), 2.87 (dd, 1H), 1.44-1.37 (m, 2H), 0.88 (t, 3H).

Step B: (2S)-ethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (38-D 1): Ethyl chloroformate (47 mg, 0.44 mmol) was added to a solution of (S)-1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline (37-D1) (47 mg, 0.087 mmol) and pyridine (35 mg, 0.44 mmol) in DCM (5 ml) at room temperature. After 2 hours the mixture was partitioned between DCM (50 mL) and sat. NaHCO₃ (50 mL). The organic layer was removed and the aqueous was extracted with DCM (1×25 mL). The combined organics were dried (MgSO₄) and concentrated. The concentrate was purified via preparative TLC to give (2S)-ethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (38-D1) (12 mg, 23%). ¹H NMR (CDCl₃) δ 7.88 (s, 1H), 7.76 (s, 2H), 7.62 (d, 1H), 7.10 (s, 1H), 7.04 (d, 1H), 6.65 (s, 1H), 4.56-4.45 (m, 1H), 4.41 (s, 3H), 4.32-4.18 (m, 2H), 3.51 (dd, 1H), 3.29 (dd, 1H), 1.33-1.20 (m, 5H), 0.81 (t, 3H). C₂₅H₂₃F₉N₆O₂ MW=610.475, observed LCMS 611.0.

### Example 12

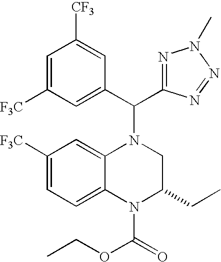

### Synthesis of (2S)-ethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (38-D2)

The diastereomer (37-D2) of (S)-1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline was converted to the carbamate derivative (38-D2) (12 mg, 22%) according to the method described in Example 11, Step B. ¹H NMR (CDCl₃) δ 7.90 (s, 1H), 7.77-7.73 (m, 3H), 7.19 (s, 1H), 7.04 (d, 1H), 6.75 (s, 1H), 4.50-4.45 (m, 1H), 4.41 (s, 3H), 4.27-4.18 (m, 2H), 3.35 (dd, 1H), 3.07 (dd, 1H), 1.58-1.43 (m, 2H), 1.30 (t, 3H) 0.78 (t, 3H). C₂₅H₂₃F₉N₆O₂ MW=610.475, observed LCMS 611.0.

### Example 13

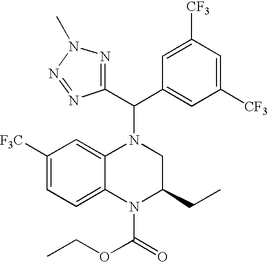

### Synthesis of (2R)-ethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (38-D3) and (38-D4)

Step A: (R)-1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline (37-D3) and (37-D4): The pair of diastereomers 37-D3 and 37-D4 for the R-ethyl series were prepared according to Example 10 and Example 11, Step A, substituting (R)-2-aminobutan-1-ol for (S)-2aminobutan-1-ol. Compounds (37-D3) (“first eluting” diastereomer) and (37-D4) (“second eluting” diastereomer) displayed the same ¹H NMR as their enantiomers 37-D1 and 37-D2.

Step B: (2R)-ethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (38-D3) and (38-D4): The pair of diastereomers (38-D3) and (38-D4) for the R-ethyl series were prepared as described in Example 11, Step B starting with (R)-1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline (37-D3) and (37-D4). These compounds displayed the same

¹H NMR as their enantiomers (38-D1) and (38-D2) above. Diastereomer (38-D3) was prepared from (37-D3) in 45% yield. C₂₅H₂₃F₉N₆O₂ MW=610.475, observed LCMS 611.0. Diastereomer (38-D4) was prepared from (37-D4) in 79% yield. C₂₅H₂₃F₉N₆O₂ MW=610.475, observed LCMS 611.0.

### Example 14

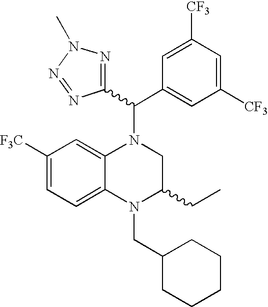

### Synthesis of 4-((3,5-bis(trifluoromethyl)phenyl)(2-methyl-2H-tetrazol-5-yl)methyl)-1-(cyclohexylmethyl)-2-ethyl-6-(trifluoromethyl)-1,2,3,4-tetrahydroquinoxaline (39-D1) and (39-D2)

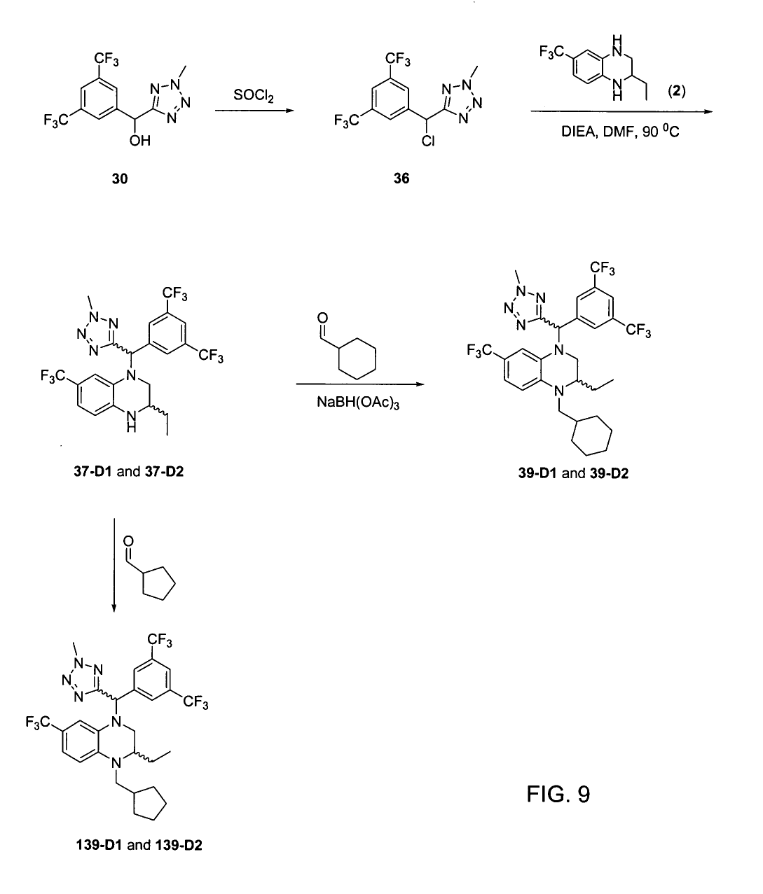

The synthetic scheme for the preparation of compounds (39-D1) and (39-D2) is shown in FIG. 9.

Step A: 1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline (36): Thionyl chloride (2.0, g, 17 mmol) was added to a solution of (3,5-bis(trifluoromethyl)phenyl)(2-methyl-2H-tetrazol-5-yl)methanol (30) (prepared as in Example 9) (5.50 g, 17 mmol) in DCM (120 mL) under argon. After 18 hours the mixture was concentrated and then rediluted in DMF (10 mL). The DMF solution was added to 2 prepared as in Example 1, Step B (1.55 g, 6.73 mmol) and DIEA (870 mg, 6.73 mmol) in DMF (30 mL). The mixture was stirred at 90° C. for 28 hours. Upon cooling the vessel contents were partitioned between EtOAc (200 mL) and water (200 mL). The phases were separated and the organic layer was washed with water (3×200 mL). The organic layer was dried and concentrated. The concentrate was purified via flash chromatography (100% hexanes to 10% EtOAc) to yield (37-D1) and (37-D2) (1.91 g, 53%) as a ˜1:1 mixture of diastereomers.

Step B: 4-((3,5-bis(trifluoromethyl)phenyl)(2-methyl-2H-tetrazol-5-yl)methyl)-1-(cyclohexylmethyl)-2-ethyl-6-(trifluoromethyl)-1,2,3,4-tetrahydroquinoxaline (39-D1 and 39-D2): To a solution of cyclohexanecarbaldehyde (32 mg, 0.29 mmol), 1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline (37-D1) and (37-D2) (31 mg, 0.058 mmol), and acetic acid (0.004, mg, 0.060 mmol) in DCE (5 mL) with 3 Å sieves present was added NaBH(OAc)₃ (61 mg, 0.29 mmol). After 16 hours the mixture was partitioned between DCM (50 mL) and water (50 mL). The organic layer was removed and the aqueous was extracted with DCM (2×50 mL). The combined organics were dried (MgSO₄) and concentrated. The concentrate was purified via preparative TLC to yield 37-D1 (2 mg, 5%, first eluting) and 37-D2 (2 mg, 5%, second eluting). 37-D1: ¹H NMR (CDCl₃) δ 7.88-7.84 (m, 3H), 7.09 (s, 1H), 7.00 (d, 1H), 6.68 (s, 1H), 7.04 (d, 1H), 6.42 (d, 1H), 4.42 (s, 3H), 3.36 (d, 1H), 3.21 (dd, 1H), 3.04-3.11 (m, 2H), 2.78 (dd, 1H), 1.78-1.48 (m, 7H), 1.35-1.08 (m, 6H), 0.94-0.84 (m, 2H), 0.31 (t, 3H). 37-D2: ¹H NMR (CDCl₃) δ 7.87 (s, 2H), 7.82 (s, 1H), 7.09 (s, 1H), 6.95 (d, 1H), 6.67 (s, 1H), 6.56 (d, 1H), 4.41 (s, 3H), 3.37 (d, 1H), 3.24 (dd, 1H), 3.15-3.10 (m, 2H), 2.97 (dd, 1H), 2.23-1.86 (m, 6H), 1.41-0.84 (m, 4H) 0.26 (t, 3H).

### Example 15

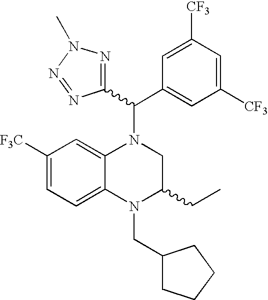

### Synthesis of 4-((3,5-bis(trifluoromethyl)phenyl)(2-methyl-2H-tetrazol-5-yl)methyl)-1-(cyclopentylmethyl)-2-ethyl-6-(trifluoromethyl)-1,2,3,4-tetrahydroquinoxaline (139-D1 and 139-D2)

The pair of diastereomers, 37-D1 and 37-D2, for the cyclopentylmethyl compounds were prepared as in Example 14 from bis(trifluoromethyl)phenyl)(2-methyl-2H-tetrazol-5-yl)methyl)-3-ethyl-7-(trifluoromethyl)-1,2,3,4-tetrahydroquinoxaline and cyclopentanecarbaldehyde to give 139-D1 (8 mg, 15%, first eluting) and 139-D2 (6, mg, 11%, second eluting). 39-D1: ¹H NMR (CDCl₃) δ 7.88 (s, 1H), 7.85 (s, 2H), 7.09 (s, 1H), 6.99 (d, 1H), 6.68 (s, 1H), 6.50 (d, 1H), 4.42 (s, 3H), 3.56 (d, 1H), 3.22-3.15 (m, 2H), 3.06 (d, 1H), 2.87 (dd, 1H), 2.32-2.26 (m, 1H), 1.80-1.10 (m, 10H), 0.33 (t, 3H). 39-D2: ¹H NMR (CDCl₃) δ 7.83 (s, 1H), 7.82 (s, 2H), 6.95 (d, 1H), 6.86 (s, 1H), 6.57 (s, 1H), 6.55 (d, 1H), 4.39 (s, 3H), 3.53 (d, 1H), 3.39-3.28 (m, 2H), 2.91 (dd, 2H), 2.37-2.27 (m, 1H), 1.75-1.45 (m, 10H) 0.67 (t, 3H).

### Example 16

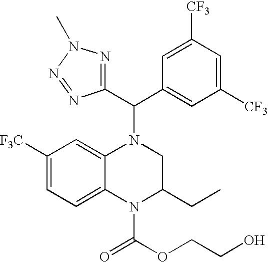

### Synthesis of 2-hydroxyethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (41)

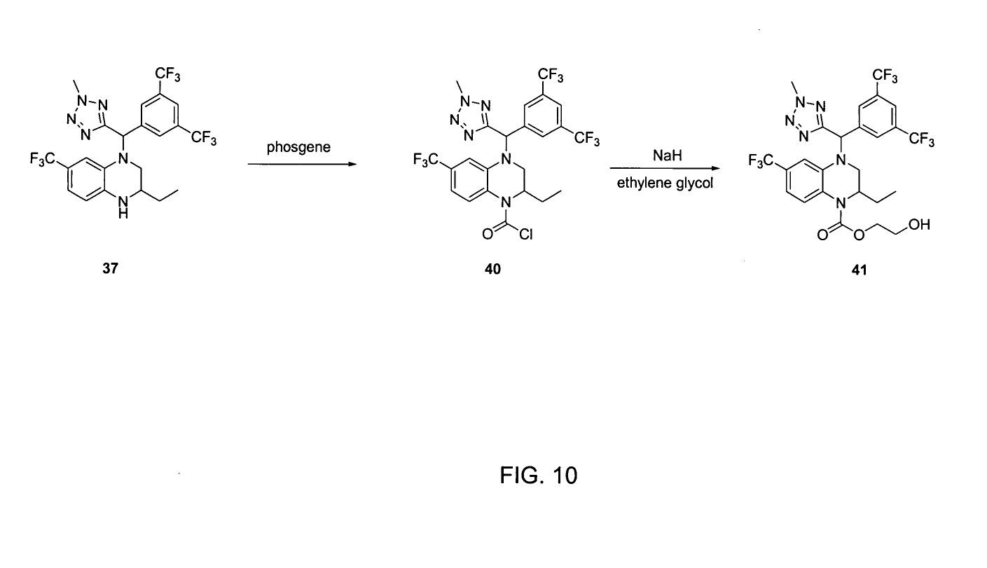

The synthetic scheme for the synthesis of compound (41) according to Example 16 is shown in FIG. 10.

Step A: 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carbonyl chloride (40): Triphosgene (9.2 mg, 0.031 mmol) was added to a 0° C. solution of 1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline (37), prepared as in Example 11, Step A (50 mg, 0.093 mmol) and DIEA (12 mg, 0.093 mmol) in CH₂Cl₂ (1 mL) to provide 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carbonyl chloride (40). The solution was stirred 4.5 hours at 0° C. at which time a 0.5 mL aliquot was removed and used without isolation in Step B.

Step B: 2-hydroxyethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (41): The aliquot from Step A containing intermediate (40) was treated with sodium hydride (60% in oil, 5 mg) and ethylene glycol (28 mg, 0.45 mmol). After being stirred overnight, the reaction was diluted with Et₂O and quenched with a saturated NaHCO₃ solution. The phases were separated and the aqueous layer was extracted once with Et₂O. The organics were combined, dried over MgSO₄, and concentrated in vacuo. The crude product was purified by column chromatography (Biotage 12M, with 4:1 Et₂O/hexanes) to afford a 9:1 diastereomeric mixture of 2-hydroxyethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (41) as a colorless film (10 mg, 36%). ¹H NMR (CDCl₃) for the major diastereomer δ 0.77-0.84 (m, 3H), 1.14-1.38 (m, 2H), 2.04 (t, 1H), 3.06-3.10 (m, 1H), 3.37-3.41 (dd, 1H), 3.84-3.87 (m, 2H), 4.32-4.34 (m, 2H), 4.41 (s, 3H), 4.47-4.51 (m, 1H), 6.75 (s, 1H), 7.05 (d, 1H), 7.19 (s, 1H), 7.72 (m, 1H), 7.76 (s, 2H), 7.91 (s, 1H).

### Example 17

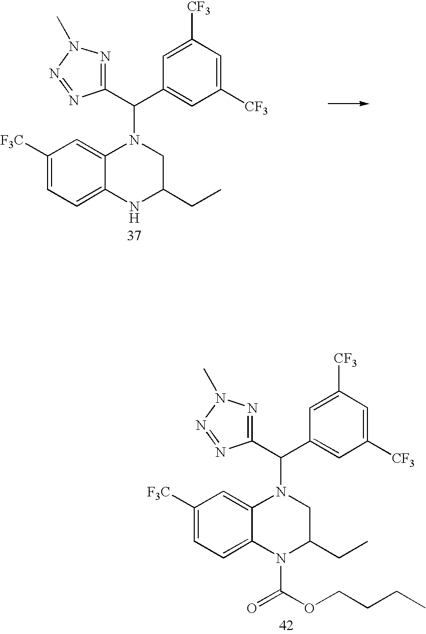

### Synthesis of butyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (42)

Butyl chloroformate (32 mg, 0.23 mmol) was added to a 0° C. solution of 1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline (25 mg, 0.046 mmol) and pyridine (18 mg, 0.023 mmol) in CH₂Cl₂ (1 mL). The solution was stirred for 30 minutes at room temperature. The reaction was diluted with 25 mL of saturated NaHCO₃ and extracted twice with CHCl₃ (25 mL each). The combined organics were dried over MgSO₄ and concentrated in vacuo. The crude product was purified by column chromatography (Biotage 12M, with 2:1 CH₂Cl₂/hexanes) to afford butyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (42) as a colorless film (17 mg, 57%). ¹H NMR (CDCl₃) δ 0.78 (t, 3H), 0.94 (t, 3H), 1.26-1.43 (m, 4H), 1.49-1.54 (m, 1H), 1.61-1.68 (m, 2H), 3.08 (d, 1H), 3.36 (dd, 1H), 4.11-4.22 (m, 1H), 4.41 (s, 3H), 4.44-4.49 (m, 1H), 6.75 (s, 1H), 7.03-7.05 (d, 1H), 7.19 (s, 1H), 7.74-7.77 (m, 3H), 7.90 (s, 1H).

### Example 18

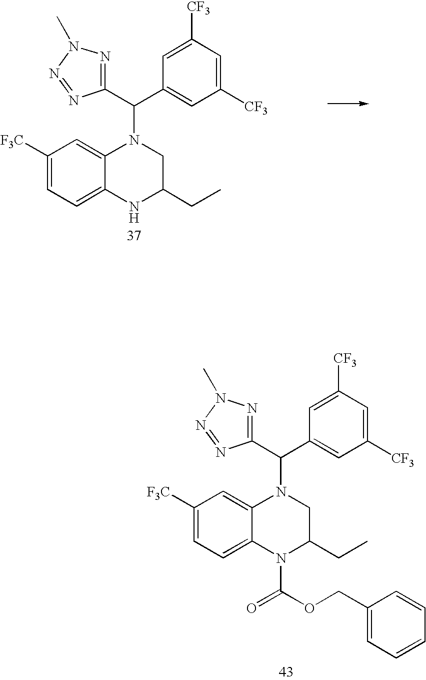

### Synthesis of benzyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (43)

Compound (43) was prepared according to the procedure for Example 17, substituting benzyl chloroformate for butyl chloroformate. The product was obtained in 58% yield. ¹H NMR (CDCl₃) δ 0.77 (t, 3H), 1.25-1.35 (m, 1H), 1.47-1.57 (m, 1H), 3.06 (dd, 1H), 3.36 (dd, 1H), 4.40 (s, 3H), 4.48-4.51 (m, 1H), 5.17 (d, 1H), 5.25 (d, 1H), 6.75 (s, 1H), 7.03 (d, 1H), 7.19 (s, 1H), 7.32-7.38 (m, 5H), 7.76 (m, 3H), 7.89 (s, 1H).

### Example 19

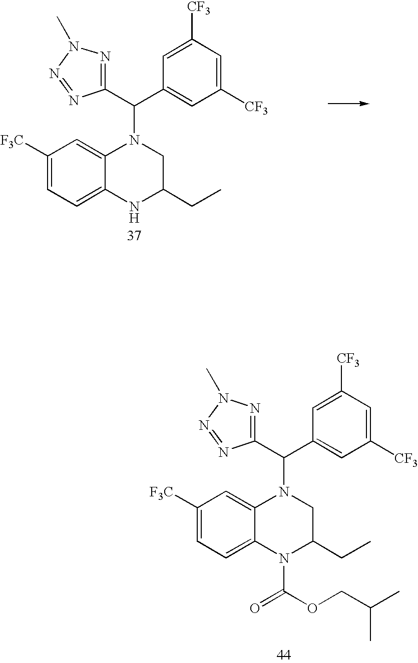

### Synthesis of isobutyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate (44)

Compound (44) was prepared according to the procedure for Example 17, substituting isobutyl chloroformate for butyl chloroformate. The product was obtained in 61% yield. ¹H NMR (CDCl₃) δ 0.79 (t, 3H), 0.94 (d, 6H), 1.25-1.34 (m, 2H), 1.47-1.57 (m, 1H), 1.91-2.01 (m, 1H), 3.08 (d, 1H) 3.37 (dd, 1H), 3.91-4.00 (m, 2H), 4.41 (s, 3H), 4.46-4.49 (m, 1H), 6.75 (s, 1H), 7.04 (d, 1H), 7.19 (s, 1H), 7.77 (s, 2H), 7.90 (s, 1H).

### Example 20

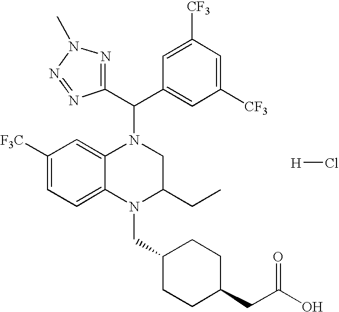

### Synthesis of 2-(trans-4-((4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)methyl)cyclohexyl)acetic acid hydrochloride (53)

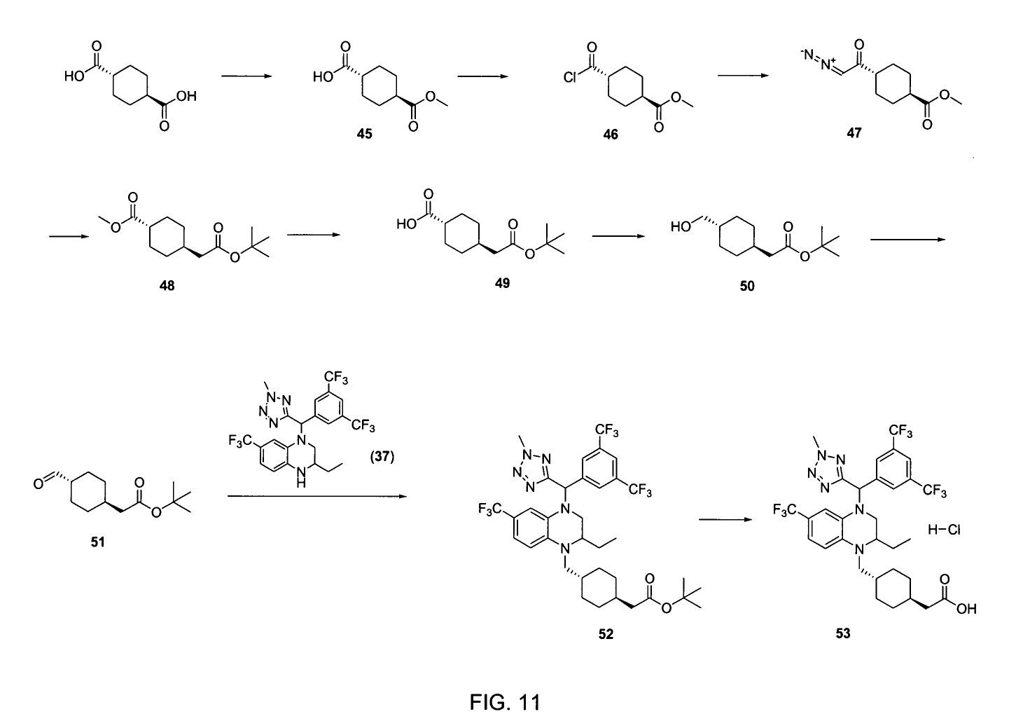

The synthetic scheme for the synthesis of compound (53) according to Example 20 is shown in FIG. 11.

Step A: trans-4-(methoxycarbonyl)cyclohexanecarboxylic acid (45): Trimethylsilyldiazomethane (2.0 M hexanes, 11.4 mL) was added to a solution of trans-1,4-cyclohexane dicarboxylic acid (3.9 g, 22.8 mmol) in 4:1 THF/MeOH (240 mL). The volatiles were removed in vacuo after the reaction was stirred overnight. The resulting crude solid was stirred in 1:1 EtOAc/hexanes (50 mL), then filtered. The filter cake was washed twice with 1:1 EtOAc/hexanes (25 mL each) and the filtrate was concentrated in vacuo. The crude product was purified by column chromatography (Biotage 40 M, with 1:2 EtOAc/hexanes containing 0.1% AcOH) to afford trans-4-(methoxycarbonyl)cyclohexanecarboxylic acid (45) as a colorless oil (1.27 g, 30%).

Step B: trans-methyl 4-(chlorocarbonyl)cyclohexanecarboxylate (46): Two drops of DMF were added to a solution of trans-4-(methoxycarbonyl)cyclohexanecarboxylic acid (200 mg, 1.07 mmol) and thionyl chloride (166 mg, 1.40 mmol) in CH₂Cl₂. The solution was stirred at room temperature overnight and the volatiles were removed in vacuo to afford trans-methyl 4-(chlorocarbonyl)cyclohexanecarboxylate (46) which was carried on to the next step.

Step C: trans-methyl 4-(diazocarbonyl)cyclohexanecarboxylate (47): Trimethylsilyldiazomethane (2.0 M hexanes, 1.6 mL) was added dropwise to a 0° C. solution of trans-methyl 4-(chlorocarbonyl)cyclohexanecarboxylate (220 mg, 1.08 mmol) in 1:1 CH₃CN/THF (10 mL). The reaction was stored at 4° C. overnight. The volatiles were removed in vacuo to afford trans-methyl 4-(diazocarbonyl)cyclohexanecarboxylate (47) which was carried on to the next step.

Step D: trans-methyl 4-(2-tert-butoxy-2-oxoethyl)cyclohexanecarboxylate (48): Silver benzoate (20 mg, 0.088 mmol) was added to a solution of crude diazoketone (190 mg, 0.88 mmol) in 1:1 dioxane/tBuOH (1 mL). The reaction was sealed and sonicated for 30 minutes. The volatiles were removed in vacuo and the crude residue was dissolved in EtOAc and washed with a saturated NaHCO₃ solution. The aqueous phase was extracted once again with EtOAc. The organics were combined, dried over MgSO₄, and concentrated in vacuo. The crude product was purified by column chromatography (Biotage 12M, with 1:4 Et₂O/hexanes) to afford trans-methyl 4-(2-tert-butoxy-2-oxoethyl)cyclohexanecarboxylate (48) as a colorless oil (67 mg, 30%, 3 steps).

Step E: trans-4-(2-tert-butoxy-2-oxoethyl)cyclohexanecarboxylic acid (49): 2.5 M KOH (1 mL) was added to a solution of the trans-methyl 4-(2-tert-butoxy-2-oxoethyl)cyclohexanecarboxylate (67 mg, 0.26 mmol) in 4:1 MeOH/H₂O (5 mL). The reaction was stirred for 30 minutes and then adjusted to pH=1 via the addition of 3% HCl. The mixture was extracted twice with 20 mL EtOAc each. The combined organics were then washed once with brine, then dried over MgSO₄ and concentrated in vacuo to afford trans-4-(2-tert-butoxy-2-oxoethyl)cyclohexanecarboxylic acid (49) as a slowly solidifying oil (65 mg, >95%).

Step F: tert-butyl 2-(trans-4-(hydroxymethyl)cyclohexyl)acetate (50): BH₃ (1 M THF, 0.30 mL) was added dropwise to a 0° C. solution of trans-4-(2-tert-butoxy-2-oxoethyl)cyclohexanecarboxylic acid (63 mg, 0.26 mmol) in THF. The reaction was stirred at 0° C. to room temperature for 15 hours, then quenched via the addition of 2:1 saturated NaHCO₃/H₂O. The mixture was partitioned between 20 mL brine and 20 mL Et₂O and the aqueous phase was extracted once with 20 mL Et₂O. The organics were dried over MgSO₄ and concentrated in vacuo to afford tert-butyl 2-(trans-4-(hydroxymethyl)cyclohexyl)acetate (50) as a colorless oil (56 mg, 89%).

Step G: tert-butyl 2-(trans-4-formylcyclohexyl)acetate (51): Dess-Martin reagent (100 mg, 0.25 mmol) was added to a 0° C. solution of tert-butyl 2-(trans-4-(hydroxymethyl)cyclohexyl)acetate (56 mg, 0.25 mmol) in CH₂Cl₂ (2.4 mL). The mixture was warmed to room temperature and stirred for 2 hours. Additional Dess-Martin reagent (24 mg, 0.065 mmol) was added and the mixture was stirred at room temperature for an additional 30 minutes. The reaction was then diluted with CH₂Cl₂ and washed with a saturated NaHCO₃ solution. The aqueous phase was extracted once with CH₂Cl₂ and the combined organics were dried over MgSO₄ and concentrated in vacuo. The crude product was purified by column chromatography (Biotage 12M, with 1:2 Et₂O/hexanes) to afford tert-butyl 2-(trans-4-formylcyclohexyl)acetate (51) as a colorless oil (29 mg, 52%).

Step H: tert-butyl 2-(trans-4-((4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)methyl)cyclohexyl)acetate (52): NaBH(OAc)₃ (44 mg, 0.21 mmol) was added to a mixture of 1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline (37), prepared as in Example 11, Step A (45 mg, 0.084 mmol), tert-butyl 2-(trans-4-formylcyclohexyl)acetate (28 mg, 0.13 mmol), and AcOH (2 drops, pipette) in DCE (1 mL). The reaction was stirred for 16 hours at room temperature and then was filtered with the filter cake being washed with CH₂Cl₂. The reaction was then washed with a saturated NaHCO₃ solution. The layers were separated and the aqueous phase was extracted once more with EtOAc. The combined organics were dried over MgSO₄ and concentrated. The crude product was purified by column chromatography (Biotage 12M, with 1:1 CH₂Cl)/hexanes) to afford tert-butyl 2-(trans-4-((4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)methyl)cyclohexyl)acetate (52) mixed with starting material. The mixture was carried on as is to Step I.

Step I: 2-(trans-4-((4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)methyl)cyclohexyl)acetic acid hydrochloride (53): 1 mL of HCl in dioxane (4 M) was added to a 0° C. solution of crude tert-butyl 2-(trans-4-((4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)methyl)cyclohexyl)acetate in THF (1.5 mL). The solution was stirred at 0° C. for 60 minutes, then 2 days at room temperature. The volatiles were removed in vacuo and the crude product was purified by column chromatography (Biotage 12M, with 1% MeOH and 0.2% AcOH in CH₂Cl₂) to afford 2-(trans-4-((4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)methyl)cyclohexyl)acetic acid hydrochloride (53) (10 mg, 33%). ¹H NMR (CDCl₃) δ 0.31 (t, 3H), 0.85-1.05 (m, 4H), 1.26-1.37 (m, 1H), 1.50-1.56 (m, 1H), 1.70-1.92 (m, 6H), 2.21 (d, 2H), 2.78 (dd, 1H), 3.05-3.10 (m, 2H), 3.22 (d, 1H), 3.40 (d, 1H), 4.41 (s, 3H), 6.42 (d, 1H), 6.68 (s, 1H), 6.99 (d, 1H), 7.09 (s, 1H), 7.85 (s, 2H), 7.88 (s, 1H).

### Example 21

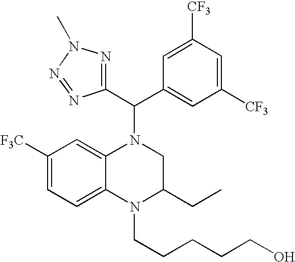

### Synthesis of 5-(4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)pentan-1-ol (58)

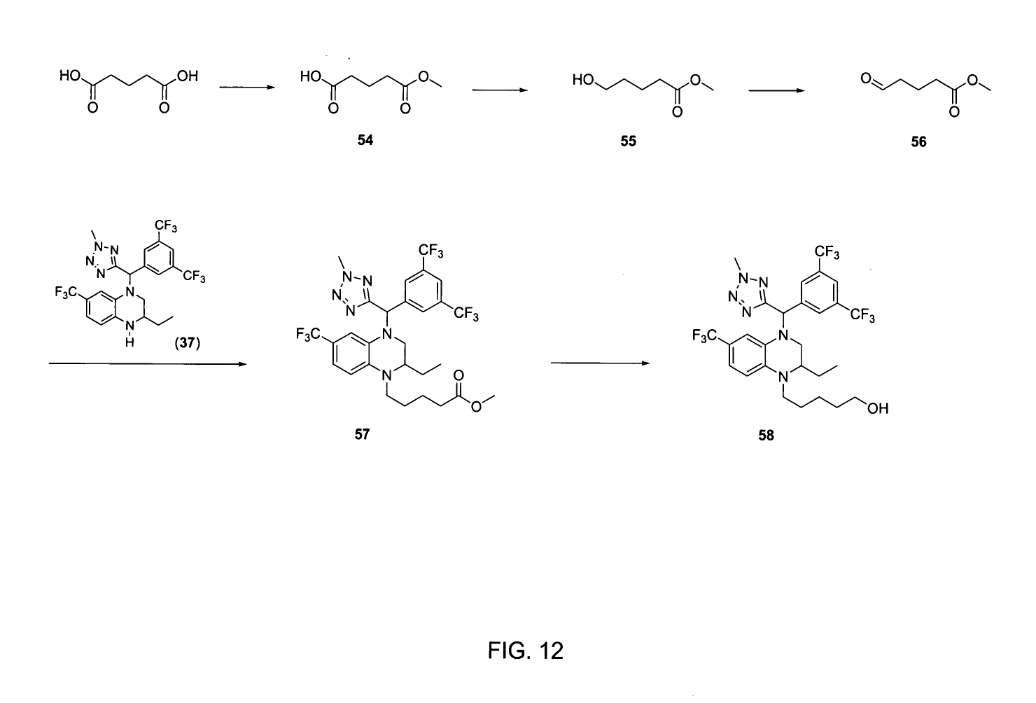

The synthetic scheme for the synthesis of compound (58) according to Example 21 is shown in FIG. 12.

Step A: 5-Methoxy-5-oxopentanoic acid (54): TMSCHN₂ (2.0 M hexanes, 3.8 mL) was added to a solution of glutaric acid (1.0 g, 7.6 mmol) in 4:1 THF/MeOH (75 mL). The solution was stirred and then the volatiles were removed in vacuo. The crude product was purified was purified by column chromatography (Biotage 40M, with 5:9 EtOAc/hexanes) to afford 5-methoxy-5-oxopentanoic acid (54) as a colorless oil (430 mg, 39%).

Step B: Methyl 5-hydroxypentanoate (55): BH₃ (1 M THF, 38 mL) was added to a 0° C. solution of 5-methoxy-5-oxopentanoic acid (5.0 g, 34 mmol) in THF (280 mL). The solution was stirred at 0° C. to room temperature overnight. The reaction was quenched via the addition of 2:1 saturated NaHCO₃/H₂O (300 mL). The mixture was diluted with 300 mL Et₂O and the phases were separated. The aqueous phase was extracted twice more with Et₂O (200 mL each). The organics were then combined and washed with brine (300 mL). The organics were dried over MgSO₄ and concentrated in vacuo to afford methyl 5-hydroxypentanoate (55) as a colorless oil (4.15 g, 92%).

Step C: Methyl 5-oxopentanoate (56): Dess-Martin reagent (3.3 g, 7.7 mmol) was added to a solution of methyl 5-hydroxypentanoate (1.0 g, 7.7 mmol) in CH₂Cl₂ (75 mL). The resulting mixture was stirred at room temperature for 1 hour, then was filtered, diluted with CH₂Cl₂, and washed with a saturated NaHCO₃ solution. The aqueous phase was extracted once with CH₂Cl₂. The organics were dried over MgSO₄ and concentrated in vacuo. The crude product was purified was purified by column chromatography (Biotage 40M, with 1:2 Et₂O/pentane) to afford methyl 5-oxopentanoate (56) as a colorless oil (0.81 g, 81%).

Step D: Methyl 5-(4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)pentanoate (57): NaBH(OAc)₃ (59 mg, 0.28 mmol) was added to a mixture of 1-((3,5-bis(trifluoromethyl)phenyl)(2-methyl-2H-tetrazol-5-yl)methyl)-3-ethyl-7-(trifluoromethyl)-1,2,3,4-tetrahydroquinoxaline (37), prepared as in Example 11, Step A (50 mg, 0.093 mmol), methyl 5-oxopentanoate (48 mg, 0.37 mmol), and AcOH (2 drops, pipette) in DCE (1 mL). The reaction was stirred for 2 hours at room temperature and then was filtered with the filter cake being washed with CH₂Cl₂. The reaction was then washed with a saturated NaHCO₃ solution. The layers were separated and the aqueous phase was extracted once more with CH₂Cl₂. The combined organics were dried over MgSO₄ and concentrated. The crude product was purified by column chromatography (Biotage 12M, with 1:1 CH₂Cl₂/hexanes) to afford methyl 5-(4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)pentanoate (57) as a colorless oil (38 mg, 63%).

Step E: 5-(4-((3,5-Bis(trifluoromethyl)phenyl)(2-methyl-2H-tetrazol-5-yl)methyl)-2-ethyl-6-(trifluoromethyl)-3, 4-dihydroquinoxalin-1(2H)-yl)pentan-1-ol (58): LiBH₄ (2 M THF, 0.036 mL) was added to a solution of methyl 5-(4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)pentanoate (24 mg, 0.037 mmol) in THF (0.8 mL). The solution refluxed for 2.5 hours, then was cooled to room temperature and quenched via the addition of a saturated NaHCO₃ solution. The mixture was extracted twice with EtOAc. The organics were dried over MgSO₄ and concentrated in vacuo. The crude product was purified by column chromatography (Biotage 12M, with 1:1 CH₂Cl₂/hexanes) to afford 5-(4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)pentan-1-ol (58) as a colorless oil (16 mg, 70%). ¹H NMR (CDCl₃) δ 0.31 (t, 3H), 1.24-1.42 (m, 6H), 1.51-1.63 (m, 2H), 3.08-3.16 (m, 4H), 3.39-3.46 (m, 1H), 3.62-3.66 (m, 2H), 4.41 (s, 3H), 6.48 (d, 1H), 6.66 (s, 1H), 7.01 (d, 1H), 7.09 (s, 1H), 7.85 (s, 2H), 7.88 (s, 1H).

### Example 22

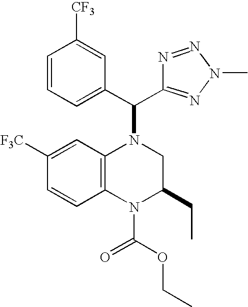

### Synthesis of ethyl 2-ethyl-4-((2-methyl-2H-tetrazol-5-yl)(3-(trifluoromethyl)phenyl)methyl)-6-(trifluoromethyl)-3,4-dihydroquinoxaline-1(2H)-carboxylate (61)

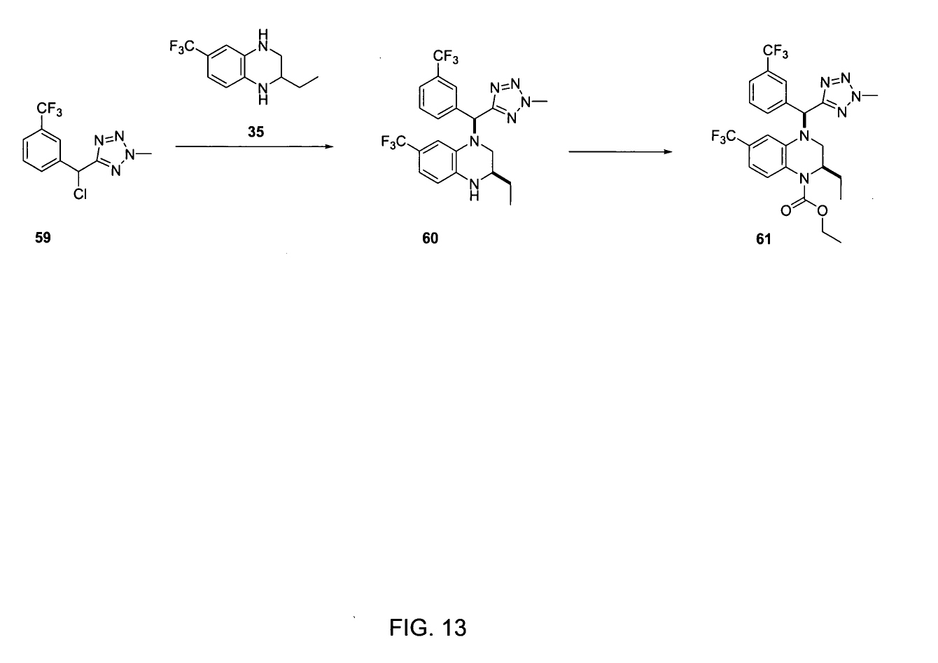

The synthetic scheme for the synthesis of compound (61) according to Example 22 is shown in FIG. 13.

Step A: 3-ethyl-1-((2-methyl-2H-tetrazol-5-yl)(3-(trifluoromethyl)phenyl)methyl)-7-(trifluoromethyl)-1,2,3,4-tetrahydroquinoxaline (60): 5-(chloro(3-(trifluoromethyl)phenyl)methyl)-2-methyl-2H-tetrazole (59) (prepared as in Example 11, Step A, substituting (3-(trifluoromethyl)phenyl)(2-methyl-2H-tetrazol-5-yl)methanol for (3,5-bis(trifluoromethyl)phenyl)(2-methyl-2H-tetrazol-5-yl)methanol) (110 mg, 0.398 mmol) and 2-ethyl-6-(trifluoromethyl)-1,2,3,4-tetrahydroquinoxaline (35), prepared as in Example 10 (275 mg, 1.19 mmol) were weighed into a 10 mL flask and diluted with anhydrous NMP (5 mL). The solution was purged with argon for 10 minutes. DIEA (69.3 μL, 0.0.398 mmol) was added and the reaction was heated to 95° C. for 18 hours. The reaction was cooled to room temperature and diluted with water (20 mL). The aqueous phase was extracted with ether (3×10 mL). The combined organics layers were washed with brine (10 mL), dried over Na₂SO₄, filtered, and concentrated. The resulting oil was purified by preparative LC plate (20% EtOAc in hexanes) to provide the desired lower R_{f} diastereomer (by TLC) of 3-ethyl-1-((2-methyl-2H-tetrazol-5-yl)(3-(trifluoromethyl)phenyl)methyl)-7-(trifluoromethyl)-1,2,3,4-tetrahydroquinoxaline (60) as a colorless film (3.6 mg, 0.0077 mmol, 1.9%).

Step B: Ethyl 2-ethyl-4-((2-methyl-2H-tetrazol-5-yl)(3-(trifluoromethyl)phenyl)methyl)-6-(trifluoromethyl)-3, 4-dihydroquinoxaline-1(2H)-carboxylate (61): To a solution of 3-ethyl-1-((2-methyl-2H-tetrazol-5-yl)(3-(trifluoromethyl)phenyl)methyl)-7-(trifluoromethyl)-1,2,3,4-tetrahydroquinoxaline (60) (3.6 mg, 0.0077 mmol) in DCM (3 mL) was added pyridine (3.1 μL, 0.038 mmol) and ethyl chloroformate (4.2 mg, 0.038 mmol). The reaction was stirred at room temperature for 1 hour, then diluted with DCM (10 mL). The organic phase was washed with 1N HCl (10 mL), then brine (10 mL), dried over Na₂SO₄, filtered, and concentrated to dryness. The resulting oil was purified by purified by Sep-pak cartridge (500 mg cartridge, 20% EtOAc in hexanes) to give ethyl 2-ethyl-4-((2-methyl-2H-tetrazol-5-yl)(3-(trifluoromethyl)phenyl)methyl)-6-(trifluoromethyl)-3,4-dihydroquinoxaline-1(2H)-carboxylate (61) as a colorless film (3.1 mg, 0.0057 mmol, 75%). ¹H NMR (400 MHz, CDCl₃) δ 0.74 (t, 3H), 1.28 (t, 3H), 1.40-1.52 (m, 2H), 3.15 (d, 1H), 3.40 (dd, 1H), 4.16-4.28 (m, 2H), 4.39 (s, 3H), 4.41-4.50 (m, 2H), 6.71 (s, 1H), 7.01 (d, 1H), 7.19 (s, 1H), 7.42-7.76 (m, 4H).

### Example 23

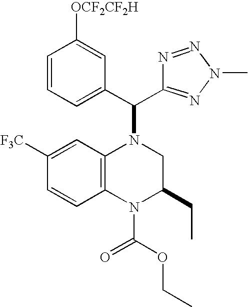

### Synthesis of Ethyl 2-ethyl-4-((2-methyl-2H-tetrazol-5-yl)(3-(111,2,2-tetrafluoroethoxy)phenyl)methyl)-6-(trifluoromethyl)-3, 4-dihydroquinoxaline-1(2H)-carboxylate (64)

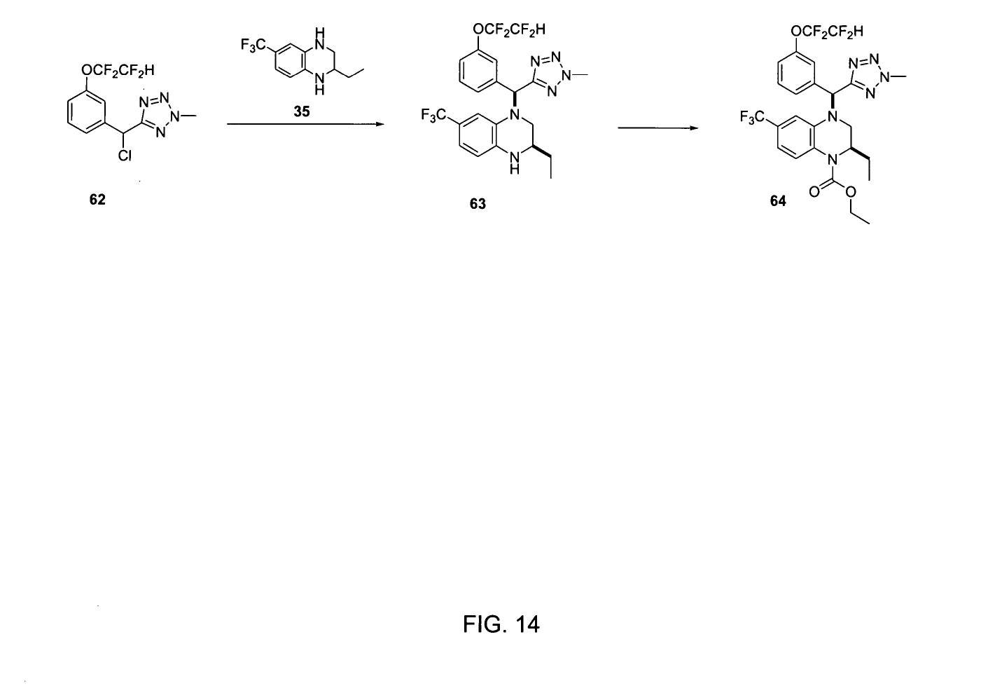

The synthetic scheme for the synthesis of compound (64) according to Example 23 is shown in FIG. 14.

3-ethyl-1-((3-(1,1,2,2-tetrafluoroethoxy)phenyl)(2H-tetrazol-5-yl)methyl)-7-(trifluoromethyl)-1.2,3,4-tetrahydroquinoxaline (63): 5-(chloro(3-(1,1,2,2-tetrafluoroethoxy)phenyl)methyl)-2-methyl-2H-tetrazole (62) (prepared as in Example 11, Step A, substituting (3-(1,1,2,2-tetrafluoroethoxy)phenyl)(2-methyl-2H-tetrazol-5-yl)methanol for (3,5-bis(trifluoromethyl)phenyl)(2-methyl-2H-tetrazol-5-yl)methanol) (276 mg, 0.850 mmol) and 2-ethyl-6-(trifluoromethyl)-1,2,3,4-tetrahydroquinoxaline (35), prepared as in Example 10, (196 mg, 0.850 mmol) were weighed into a 10 mL flask and diluted with anhydrous NMP (4 mL). The solution was purged with argon for 20 minutes. Pyridine (67.2 mg, 0.850 mmol) was added and the reaction was heated to 95° C. for 18 hours. The reaction was cooled to room temperature and diluted with water (20 mL). The aqueous phase was extracted with ether (3×10 mL). The combined organics layers were washed with brine (10 mL), dried over Na₂SO₄, filtered, and concentrated. The resulting brown oil was purified by column chromatography (Biotage 12m, gradient 5% EtOAc in hexanes to 15% EtOAc in hexanes) to provide two diastereomers of 3-ethyl-1-((3-(1,1,2,2-tetrafluoroethoxy)phenyl)(2H-tetrazol-5-yl)methyl)-7-(trifluoromethyl)-1,2,3,4-tetrahydroquinoxaline (63) as colorless films (28.6 mg, 0.055 mmol, 6.4%).

Step B: Ethyl 2-ethyl-4-((2-methyl-2H-tetrazol-5-yl)(3-(111,2,2-tetrafluoroethoxy)phenyl)methyl)-6-(trifluoromethyl)-3,4-dihydroquinoxaline-1(2H)-carboxylate (64): To a solution of 3-ethyl-1-((3-(1,1,2,2-tetrafluoroethoxy)phenyl)(2H-tetrazol-5-yl)methyl)-7-(trifluoromethyl)-1,2,3,4-tetrahydroquinoxaline (63) (28.6 mg, 0.0552 mmol) in DCM (2 mL) was added DIEA (48 μL, 0.276 mmol) and ethyl chloroformate (29.9 mg, 0.276 mmol). The reaction was stirred at room temperature for 1 hour, then diluted with DCM (10 mL). The organic phase was washed with 1N HCl (10 mL), then brine (10 mL), dried over Na₂SO₄, filtered, and concentrated to dryness. The resulting oil was purified by Sep-pak cartridge (20% EtOAc in hexanes, 500 mg cartridge) to provide a single diastereomer of ethyl 2-ethyl-4-((2-methyl-2H-tetrazol-5-yl)(3-(1,1,2,2-tetrafluoroethoxy)phenyl)methyl)-6-(trifluoromethyl)-3,4-dihydroquinoxaline-1(2H)-carboxylate (64) as colorless oil (12 mg 0.020 mmol, 37%). ¹H NMR (400 MHz, CDCl₃) δ 0.75 (t, 3H), 0.23-1.33 (m, 5H), 1.42-1.50 (m, 1H), 3.18 (d, 1H), 3.42 (dd, 1H), 4.15-4.28 (m, 2H), 4.38 (s, 3H), 4.44-4.49 (m, 1H), 6.65 (s, 1H), 6.98 (d, 1H), 7.16 (br s, 1H), 7.22 (d, 1H), 7.40 (t, 1H), 7.72 (br d, 1H).

### Example 24

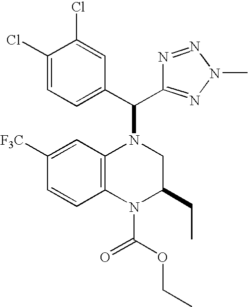

### Synthesis of Ethyl-4-((3,4-dichlorophenyl)(2-methyl-2H-tetrazol-5-yl)methyl)-2-ethyl-6-(trifluoromethyl)-3,4-dihydroquinoxaline-1(2H)-carboxylate (68)

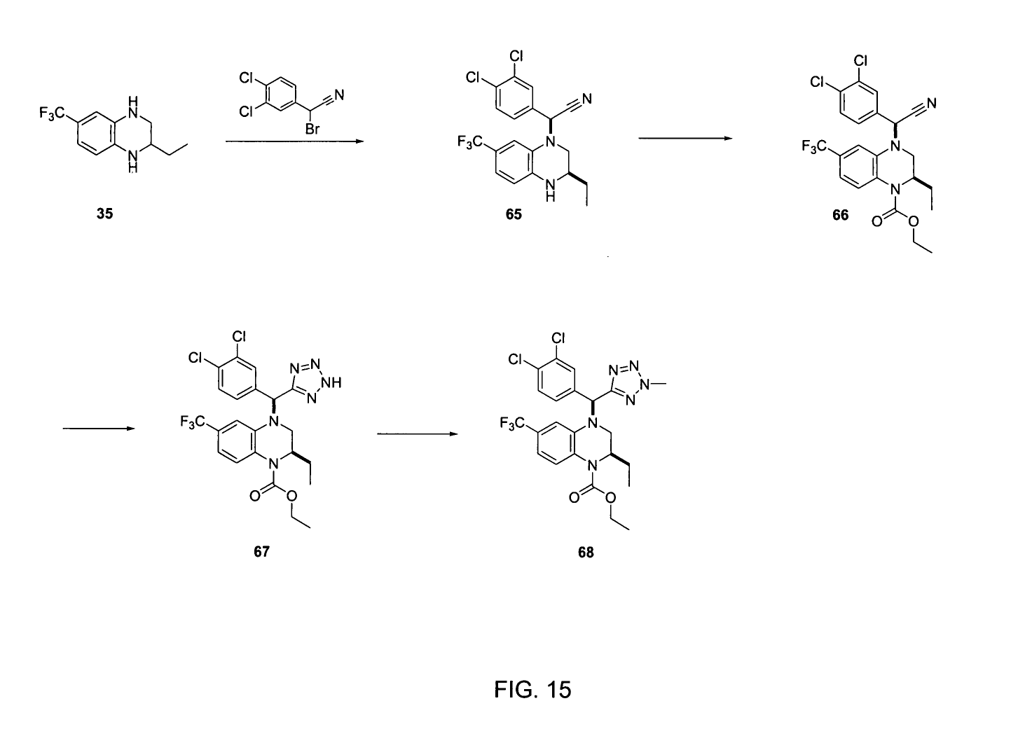

The synthetic scheme for the synthesis of compound (68) according to Example 24 is shown in FIG. 15.

Step A: 2-(3,4-dichlorophenyl)-2-(3-ethyl-7-(trifluoromethyl)-3,4-dihydroquinoxalin-1(2H)-yl)acetonitrile (65): 2-Ethyl-6-(trifluoromethyl)-1,2,3,4-tetrahydroquinoxaline (35), prepared as in Example 10 (105 mg, 0.457 mmol) and DIEA (99.6 μL, 0.572 mmol) were weighed into a 10 mL round bottom flask. Anhydrous DMF (2 mL) was added and the resulting solution was purged with argon for 5 minutes. 2-bromo-2-(3,4-dichlorophenyl)acetonitrile (202 mg, 0.762 mmol) was added as a solid and argon bubbling was continued for another 5 minutes. The reaction was stirred at room temperature overnight, then partitioned between water (30 mL) and Et₂O (30 mL). The organic phase was washed with water (30 mL), then dried over MgSO₄, filtered, and concentrated. The resulting oil was purified by column chromatography (Biotage 12m, 1:2 CH₂Cl₂/hexanes) to give 2-(3,4-dichlorophenyl)-2-(3-ethyl-7-(trifluoromethyl)-3,4-dihydroquinoxalin-1(2H)-yl)acetonitrile (65) as a yellow oil.

Step B: Ethyl 4-(cyano(3,4-dichlorophenyl)methyl)-2-ethyl-6-(trifluoromethyl)-3,4-dihydroquinoxaline-1(2H)-carboxylate (66): The crude yellow oil (65) from Step A was dissolved in a DCM solution (2 mL) containing pyridine (71 mg, 0.90 mmol) and 1 mL of ethyl chloroformate (98 mg, 0.90 mmol). The reaction was stirred at room temperature overnight, then diluted with DCM (20 mL), and washed with 1N HCl (2×10 mL). The organic phase was dried over Na₂SO₄, and concentrated. The resulting oil was purfied by column chromatography (Biotage 12m, 1:1 CH₂Cl₂/hexanes) to yield ethyl 4-(cyano(3,4-dichlorophenyl)methyl)-2-ethyl-6-(trifluoromethyl)-3,4-dihydroquinoxaline-1 (2H)-carboxylate (66) as a colorless oil (62 mg, 0.128 mmol, 20%).

Step C: Ethyl 4-((3,4-dichlorophenyl)(2H-tetrazol-5-yl)methyl)-2-ethyl-6-(trifluoromethyl)-3,4-dihydroquinoxaline-1(2H)-carboxylate (67): To a solution of ethyl 4-(cyano(3,4-dichlorophenyl)methyl)-2-ethyl-6-(trifluoromethyl)-3,4-dihydroquinoxaline-1 (2H)-carboxylate (66) (61 mg, 0.13 mmol) in anhydrous DMF (3 mL) was added NaN₃ (41 mg, 0.63 mmol) and NH₄Cl (34 mg, 0.63 mmol). The reaction was heated to 75° C. for 3 hours, then cooled to room temperature and diluted with saturated NaHCO₃ (10 mL). The aqueous phase was extracted with DCM (3×10 mL). The combined organic layers were washed with brine (10 mL), dried over Na₂SO₄, and concentrated to dryness. The resulting oil was purified by reverse phase LC (Horizon system, column 12L, gradient 30% acetonitrile in water to 100% acetonitrile) to provide two diastereomers of ethyl 4-((3,4-dichlorophenyl)(2H-tetrazol-5-yl)methyl)-2-ethyl-6-(trifluoromethyl)-3,4-dihydroquinoxaline-1(2H)-carboxylate (67) as colorless oils. The desired lower R_{f} fraction (by TLC) provided 6.8 mg (0.013 mmol, 10% yield).

Step D: Ethyl-4-((3,4-dichlorophenyl)(2-methyl-2H-tetrazol-5-yl)methyl)-2-ethyl-6-(trifluoromethyl)-3,4-dihydroquinoxaline-1(2H)-carboxylate (68): To a solution of ethyl-4-((3,4-dichlorophenyl)(2H-tetrazol-5-yl)methyl)-2-ethyl-6-(trifluoromethyl)-3,4-dihydroquinoxaline-1(2H)-carboxylate (66; lower Rf fraction) (6.8 mg, 0.013 mmol) in THF/MeOH (4:1, 5 mL) was added TMS-diazomethane (13 μL, 0.026 mmol, 2.0 M in DCM). The reaction was stirred at room temperature for 15 minutes, then concentrated to dryness. The resulting oil was purified by Sep-pak cartridge (20% EtOAc in hexanes, 500 mg cartridge) to provide 1.7 mg (0.003 mmol, 24% yield) of ethyl 4-((3,4-dichlorophenyl)(2-methyl-2H-tetrazol-5-yl)methyl)-2-ethyl-6-(trifluoromethyl)-3,4-dihydroquinoxaline-1(2H)-carboxylate (68) as a colorless oil (1.7 mg, 0.0031 mmol, 24%). ¹H NMR (400 MHz, CDCl₃) δ 0.80 (t, 3H), 1.30-1.31 (m, 5H), 1.45-1.50 (m, 1H), 3.16 (d, 1H), 3.39 (dd, 1H), 4.23-4.28 (m, 2H), 4.38 (s, 3H), 4.42-4.49 (m, 1H), 6.60 (s, 1H), 6.99 (d, 1H), 7.11-7.14 (m, 1H), 7.37 (s, 1H), 7.45 (d, 1H), 7.47 (br d, 1H).

### Example 25

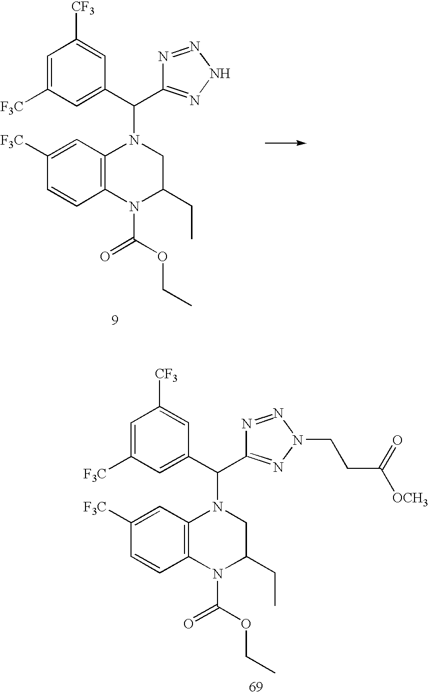

### Synthesis of Ethyl 4-((3,5-bis(trifluoromethyl)phenyl)(2-(3-methoxy-3-oxopropyl)-2H-tetrazol-5-yl)methyl)-2-ethyl-6-(trifluoromethyl)-3, 4-dihydroquinoxaline-1(2H)-carboxylate

To a solution of ethyl 4-((3,5-bis(trifluoromethyl)phenyl)(2H-tetrazol-5-yl)methyl)-2-ethyl-6-(trifluoromethyl)-3,4-dihydroquinoxaline-1(2H)-carboxylate (9), prepared as in Example 2 (50 mg, 0.084 mmol) in dichloroethane (1 mL) was added DIEA (16 mg, 0.13 mmol) and methyl 3-bromopropanoate (21 mg, 0.13 mmol). The reaction was heated to 60° C. for 14 hours, then cooled and concentrated to dryness. The resulting oil was purified by preparative LC plate (20% EtOAc in hexanes) to provide ethyl 4-((3,5-bis(trifluoromethyl)phenyl)(2-(3-methoxy-3-oxopropyl)-2H-tetrazol-5-yl)methyl)-2-ethyl-6-(trifluoromethyl)-3,4-dihydroquinoxaline-1(2H)-carboxylate (69) as a mixture of diastereomers. (9.0 mg, 0.084 mmol, 16%) ¹H NMR (400 MHz, CDCl₃) δ 0.74-0.84 (m, 3H), 1.22-1.36 (m, 5H), 1.47-1.53 (m, 1H), 3.02-3.14 (m, 3H), 3.27-3.52 (m, 1H), 3.65 (d, 3H), 4.18-4.30 (m, 2H), 4.43-4.57 (m, 1H), 4.92-5.01 (m, 2H), 6.65 (s, 0.6H), 6.74 (s, 0.4H), 6.98-7.10 (m, 1H), 7.17 (s, 1H), 7.75 (s, 3H), 7.89 (br d, 1H).

### Example 26

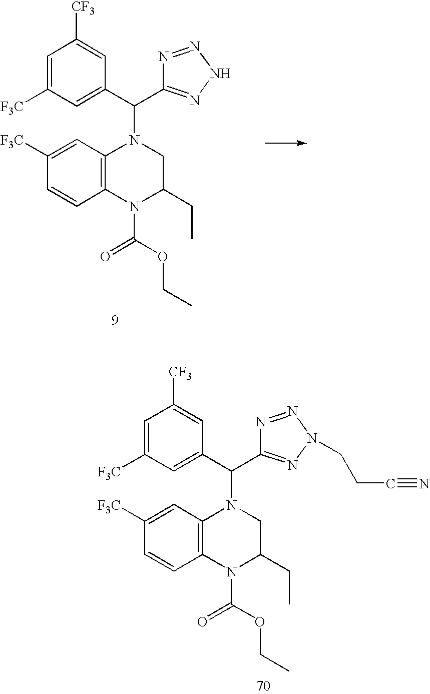

### Synthesis of Ethyl 4-((3,5-bis(trifluoromethylphenyl)(2-(2-cyanoethyl)-2H-tetrazol-5-yl)methyl)-2-ethyl-6-(trifluoromethyl)-3,4-dihydroquinoxaline-1(2H)-carboxylate (70)

To a solution of ethyl 4-((3,5-bis(trifluoromethyl)phenyl)(2H-tetrazol-5-yl)methyl)-2-ethyl-6-(trifluoromethyl)-3,4-dihydroquinoxaline-1(2H)-carboxylate (9) prepared as in Example 2 (50 mg, 0.084 mmol) in dichloroethane (1 mL) was added DIEA (16 mg, 0.13 mmol) and 3-bromopropanenitrile (17 mg, 0.13 mmol). The reaction was heated to 60° C. for 14 hours, then the reaction was cooled to room temperature, and concentrated to dryness. The resulting oil was purified by preparative LC plate (20% EtOAc in hexanes) to provide ethyl 4-((3,5-bis(trifluoromethyl)phenyl)(2-(2-cyanoethyl)-2H-tetrazol-5-yl)methyl)-2-ethyl-6-(trifluoromethyl)-3,4-dihydroquinoxaline-1(2H)-carboxylate (70) as a mixture of diastereomers (6.0 mg, 0.084 mmol, 11%). ¹H NMR (400 MHz, CDCl₃) δ 0.74-0.86 (m, 3H), 1.52-1.72 (m, 6H), 3.07 (d, 0.5H), 3.15 (t, 2H), 3.34-3.51 (m, 1.5H), 4.19-4.29 (m, 2H), 4.48-4.55 (m, 1H), 4.95-5.00 (m, 2H), 6.67 (s, 0.5H), 6.78 (s, 0.5H), 7.04-7.06 (m, 1.7H), 7.18 (s, 0.5H), 7.75 (s, 3H), 7.90 (d, 1H).

### Example 27

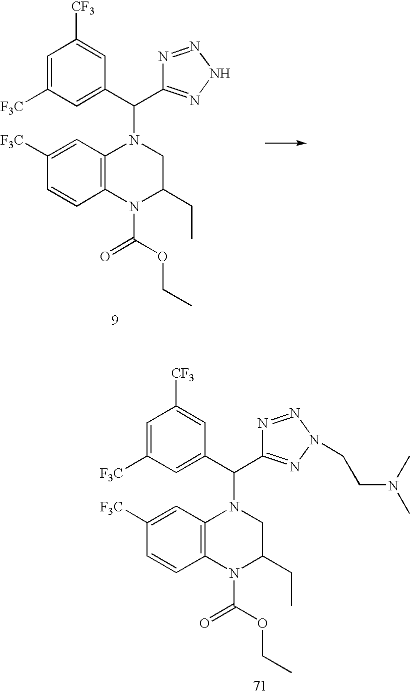

### Synthesis of Ethyl 4-((3,5-bis(trifluoromethyl)phenyl)(2-(2-(dimethylamino)ethyl)-2H-tetrazol-5-yl)methyl)-2-ethyl-6-(trifluoromethyl)-3,4-dihydroquinoxaline-1(2H)-carboxylate (71)

To a solution of ethyl 4-((3,5-bis(trifluoromethyl)phenyl)(2H-tetrazol-5-yl)methyl)-2-ethyl-6-(trifluoromethyl)-3,4-dihydroquinoxaline-1(2H)-carboxylate (9) prepared as in Example 2 (50 mg, 0.084 mmol) in dichloroethane (1 mL) was added DIEA (16 mg, 0.13 mmol) and 2-chloro-N,N-dimethylethanamine (9 mg, 0.08 mmol). The reaction was heated to 80° C. for 14 hours. The reaction was concentrated to dryness. The resulting oil was purified by preparative LC plate (50% EtOAc in hexanes) to provide ethyl 4-((3,5-bis(trifluoromethyl)phenyl)(2-(2-(dimethylamino)ethyl)-2H-tetrazol-5-yl)methyl)-2-ethyl-6-(trifluoromethyl)-3,4-dihydroquinoxaline-1(2H)-carboxylate (71) as a mixture of diastereomers (18 mg, 0.027 mmol, 32%). ¹H NMR (400 MHz, CDCl₃) δ 0.78 (dt, 3H), 1.21-1.34 (m, 5H), 1.46-1.55 (m, 1H), 2.23 (d, 6H), 2.89-2.92 (m, 2H), 3.06 (dd, 0.7H), 3.28-3.53 (m, 1.3H), 4.19-4.28 (m, 2H), 4.46-4.51 (m, 1H), 4.68-4.80 (m, 2H), 6.65 (s, 0.3H), 6.74 (s, 0.6H), 7.02 (t, 1H), 7.10 (s, 0.3H), 7.18 (s, 0.6H), 7.75 (s, 2H), 7.89 (d, 1H).

### Example 28

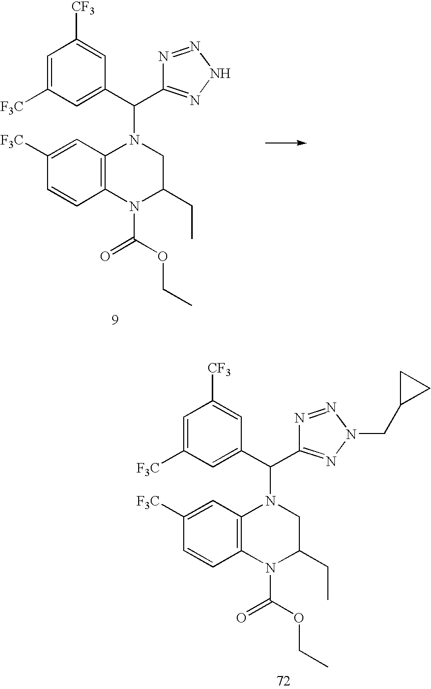

### Synthesis of Ethyl 4-((3,5-bis(trifluoromethyl)phenyl)(2-(cyclopropylmethyl)-2H-tetrazol-5-yl)methyl)-2-ethyl-6-(trifluoromethyl)-3,4-dihydroquinoxaline-1(2H)-carboxylate (72)

To a solution of ethyl 4-((3,5-bis(trifluoromethyl)phenyl)(2H-tetrazol-5-yl)methyl)-2-ethyl-6-(trifluoromethyl)-3,4-dihydroquinoxaline-1(2H)-carboxylate (9) prepared as in Example 2 (50 mg, 0.084 mmol) in dichloroethane (3 mL) was added DIEA (16 mg, 0.13 mmol) and 2-bromoacetamide (17 mg, 0.13 mmol). The reaction was heated to 60° C. for 14 hours. The reaction was concentrated to dryness. The resulting oil was purified by preparative LC plate (20% EtOAc in hexanes) to provide ethyl 4-((3,5-bis(trifluoromethyl)phenyl)(2-(cyclopropylmethyl)-2H-tetrazol-5-yl)methyl)-2-ethyl-6-(trifluoromethyl)-3,4-dihydroquinoxaline-1(2H)-carboxylate (72) as a mixture of diastereomers. (12 mg, 0.018 mmol, 22%). ¹H NMR (400 MHz, CDCl₃) δ 0.48-0.52 (m, 2H), 0.66-0.70 (d, 2H), 0.78 (dt, 3H), 1.19-1.56 (m, 7H), 3.09 (d, 0.6H), 3.28-3.40 (m, 1H), 3.52 (d, 0.4H), 4.18-4.29 (m, 2H), 4.42-4.58 (m, 3H), 6.67 (s, 0.4H), 6.75 (s, 0.6H), 7.03 (t, 1H), 7.12 (s, 0.4H), 7.20 (s, 0.6H), 7.78 (s, 2H), 7.89 (d, 1H).

### Example 29

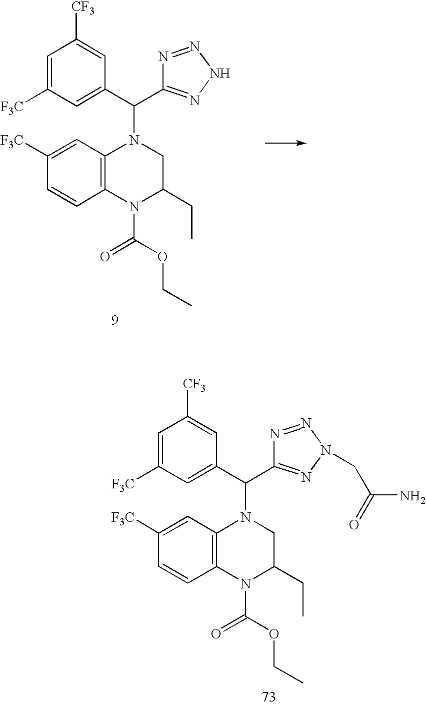

### Synthesis of Ethyl 4-((3,5-bis(trifluoromethyl)phenyl)(2-(2-amino-2-oxoethyl)-2H-tetrazol-5-yl)methyl)-2-ethyl-6-(trifluoromethyl)-3,4-dihydroquinoxaline-1(2H)-carboxylate (73)

To a solution of ethyl 4-((3,5-bis(trifluoromethyl)phenyl)(2H-tetrazol-5-yl)methyl)-2-ethyl-6-(trifluoromethyl)-3,4-dihydroquinoxaline-1(2H)-carboxylate (9) prepared as in Example 2 (50 mg, 0.084 mmol) in dichloroethane (3 mL) was added DIEA (16 mg, 0.13 mmol) and 2-bromoacetamide (17 mg, 0.13 mmol). The reaction was heated to 60° C. for 14 hours. The reaction was concentrated to dryness. The resulting oil was purified by preparative LC plate (20% ether in hexanes) to provide a single diastereomer of ethyl 4-((3,5-bis(trifluoromethyl)phenyl)(2-(2-amino-2-oxoethyl)-2H-tetrazol-5-yl)methyl)-2-ethyl-6-(trifluoromethyl)-3,4-dihydroquinoxaline-1(2H)-carboxylate (73) as a colorless oil (24 mg, 0.037 mmol, 44%). ¹H NMR (400 MHz, CDCl₃) δ 0.77 (t, 2H), 1.24-1.34 (dt, 3H), 1.48-1.55 (m, 1H), 3.05 (d, 1H), 3.38 (dd, 1H), 4.08-4.27 (m, 2H), 4.45-4.51 (m, 1H), 5.39-5.41 (d, 2H), 5.50-5.75 (br d, 2H) 6.80 (s, 1H), 7.05 (d, 1H), 7.18 (s, 1H), 7.79 (s, 2H), 7.92 (s, 1H).

### Example 30

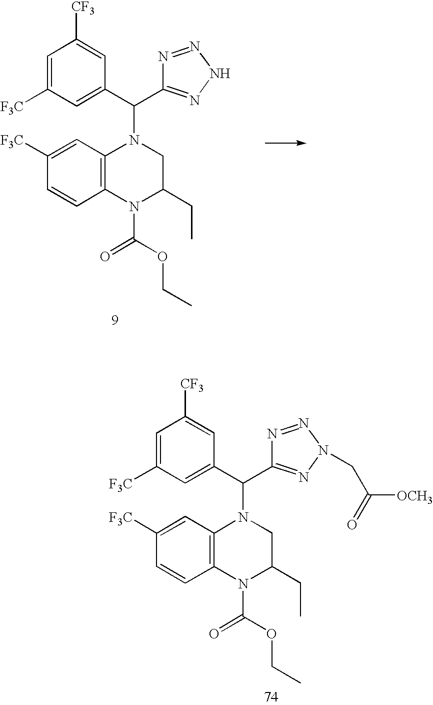

### Synthesis of Ethyl-4-((3,5-bis(trifluoromethyl)phenyl)(2-(2-hydroxyethyl)-2H-tetrazol-5-yl)methyl)-2-ethyl-6-(trifluoromethyl)-3,4-dihydroquinoxaline-1(2H)-carboxylate (74)

To a solution of ethyl 4-((3,5-bis(trifluoromethyl)phenyl)(2H-tetrazol-5-yl)methyl)-2-ethyl-6-(trifluoromethyl)-3,4-dihydroquinoxaline-1(2H)-carboxylate (9) prepared as in Example 2 (48 mg, 0.080 mmol) in dichloroethane (3 mL) was added DIEA (16 mg, 0.12 mmol) and methyl bromoacetate (18 mg, 0.12 mmol). The reaction was heated to 60° C. for 1.5 hours, then cooled to room temperature, and concentrated to dryness. The resulting oil was purified by flash chromatography (Biotage 12m, 40% ether in hexanes) to provide ethyl 4-((3,5-bis(trifluoromethyl)phenyl)(2-(2-methoxy-2-oxoethyl)-2H-tetrazol-5-yl)methyl)-2-ethyl-6-(trifluoromethyl)-3,4-dihydroquinoxaline-1(2H)-carboxylate (74) as a mixture of diastereomers (28.2 mg, 0.042, 52%).

### Example 31

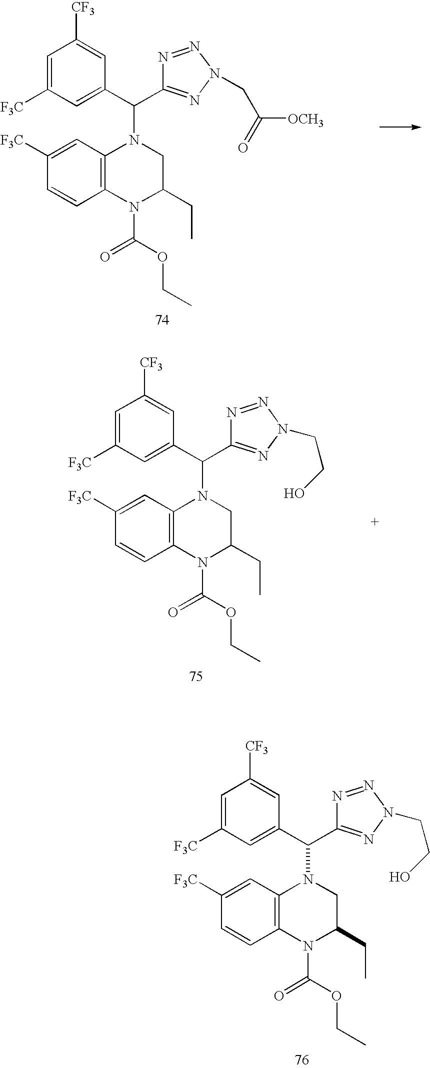

### Synthesis of Ethyl-4-((3, 5-bis(trifluoromethyl)phenyl)(2-(2-hydroxyethyl)-2H-tetrazol-5-yl)methyl)-2-ethyl-6-(trifluoromethyl)-3,4-dihydroquinoxaline-1(2H)-carboxylate (75) and (76)

To a flame dried, nitrogen purged 25 mL flask, was added ethyl 4-((3,5-bis(trifluoromethyl)phenyl)(2-(2-methoxy-2-oxoethyl)-2H-tetrazol-5-yl)methyl)-2-ethyl-6-(trifluoromethyl)-3,4-dihydroquinoxaline-1(2H)-carboxylate (74) prepared as in Example 30 (8.9 mg, 0.013 mmol) and anhydrous THF (2 mL). A solution of LiBH₄ (6.7 μL, 0.013 mmol, 2M in THF) was added and the reaction was heated to reflux for 1 hour. The reaction was cooled to room temperature and quenched with 1N HCl (20 mL). After 10 minutes of stirring, the aqueous phase was extracted with EtOAc (2×10 mL). The EtOAc layer was washed with brine (20 mL), dried over Na₂SO₄, filtered, and concentrated. The crude oil was purified by C-18 column chromatography, (Horizon 12L, gradient: 5% acetonitrile/water to 95% acetonitrile/water) to give two diastereomers of ethyl 4-((3,5-bis(trifluoromethyl)phenyl)(2-(2-hydroxyethyl)-2H-tetrazol-5-yl)methyl)-2-ethyl-6-(trifluoromethyl)-3,4-dihydroquinoxaline-1(2H)-carboxylate (75) and (76) as colorless oils. Higher R_{f}diastereomer (by TLC) (3.1 mg, 0.0047 mmol, 36%): ¹H NMR (400 MHz, CDCl₃) δ 0.81 (t, 3H), 1.22-1.33 (m, 7H), 1.65 (br s, 1H), 3.31 (dd, 1H), 3.52 (d, 1H), 4.20-4.31 (m, 3H), 4.50-4.56 (m, 1H), 4.80-4.83 (m, 2H), 6.66 (s, 1H), 7.02 (d, 1H), 7.09 (s, 1H), 7.78 (s, 2H), 7.88 (s, 1H); Lower R_{f} diastereomer (by TLC) (2.7 mg, 0.0041 mmol, 31%): ¹H NMR (400 MHz, CDCl₃) δ 0.0.78 (t, 3H), 1.26-1.34 (m, 6H), 1.48-1.56 (m, 1H), 1.70 (br s, 1H), 3.07 (d, 1H), 3.36 (dd, 1H), 4.18-4.26 (m, 3H), 4.47-4.49 (m, 1H), 4.79-4.83 (m, 2H), 6.76 (s, 1H), 7.04 (d, 1H), 7.20 (s, 1H), 7.80 (s, 2H), 7.91 (s, 1H).

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A compound having the Formula: 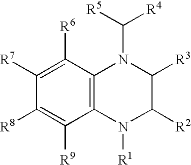
and metabolites, solvates, tautomers, resolved enantiomers, diastereomers, pharmaceutically acceptable salts and pharmaceutically acceptable prodrugs thereof, wherein:
R¹ is Zₙ-(C═O)OR¹⁰, Zₙ-(C═O)R¹⁰, Zₙ(C═O)Zₙ(C═O)OR¹⁰, Zₙ-NR¹OR¹, Zₙ-(C═O)NR¹⁰R¹¹, ZₙSOR¹⁰, Zₙ-SO₂R¹⁰, alkyl, allyl, alkenyl, alkynyl, heteroalkyl, heteroallyl, heteroalkenyl, heteroalkynyl, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, Zₙ-cycloalkyl, Zₙ-heterocycloalkyl and Zₙ-Ar are optionally substituted with one or more groups independently selected from F, Zₙ-COOR¹⁰, ZₙOR¹⁰, Zₙ-NR¹⁰R¹¹, Zₙ-(C═O)NR¹⁰R¹¹, oxo and alkyl;
R² and R³ are independently H, OH, F, Cl, Br, I, CF₃, Zₙ-NR¹⁰R¹¹, Zₙ-NR¹⁰(C═O)R¹¹, Zₙ-SO₂R¹⁰, Zₙ-SOR¹⁰, Zₘ-SR¹⁰, Zₙ-OR¹¹, Zₙ-(C═O)R¹⁰, Zₙ-(C═O)OR¹⁰, Zₙ-O(C═O)R¹⁰, alkyl, allyl, alkenyl, alkynyl, heteroalkyl, heteroallyl, heteroalkenyl, heteroalkynyl, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar are optionally substituted with one or more groups independently selected from OR¹⁰ and SR¹⁰;
or R¹ and R² together with the atoms to which they are attached form a substituted or unsubstituted, saturated or partially unsaturated 5 or 6-membered heterocyclic ring;
R⁴ is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, OR′, SR′F, Cl, Br, I, CN and NO₂, wherein said alkyl is optionally substituted with one or more groups independently selected from C(═O)OR′, CN, NR′R″, C(═O)NR′R″, cycloalkyl, OH, F and alkyl;
R⁵ is heteroaryl optionally substituted with one or more groups independently selected from H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₙ-NR′R″, Zₙ-C(═O)NR′R″, Zₙ-OR′, F, Cl, Br or I;
R⁶ and R⁷, R⁷ and R⁸ and R⁹ are independently H, OH, F, Cl, Br, I, CF₃, OCF₃, OCF₂H, Zₙ-NR¹⁰R¹¹, Zₙ-(C═O)NR¹⁰R¹¹, Zₙ-SO₂R¹⁰, Zₙ-SOR¹⁰, Zₙ-SR¹⁰, Zₙ-OR¹⁰, Zₙ-(C═O)R¹⁰, Zₙ-(C═O)OR¹⁰, Zₙ-O—(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar,
or R⁶ and R⁷, R⁷ and R⁸, and/or R⁸ and R⁹ together with the atoms to which they are attached form a carbocyclic or heterocyclic ring, wherein said carbocyclic and heterocyclic rings are optionally substituted with one or more groups independently selected from alkyl and F;
R¹⁰ and R¹¹ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, cycloalkyl, heterocycloalkyl and Ar are optionally substituted with one or more groups independently selected from alkyl, OR′ and Ar,
or R¹⁰ and R¹¹ together with the atoms to which they are attached form a substituted or unsubstituted, saturated or partially unsaturated 5 or 6-membered heterocyclic ring;
Z is alkylene having from 1 to 4 carbons, or alkenylene or alkynylene each having from 2 to 4 carbons, wherein said alkylene, alkenylene and alkynylene are optionally substituted;
Ar is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, OR′, SR′F, Cl, Br, I, CN and NO₂, wherein said alkyl is optionally substituted with one or more groups independently selected from C(═O)OR′, CN, NR′R″, C(═O)NR′R″, cycloalkyl, OR′, F and alkyl;
R′ and R″ are independently H or C₁-C₁₀ alkyl, wherein said alkyl is optionally substituted with one or more F;
m is 1 or 2; and
n is 0, 1, or 2.

2. The compound of claim 1, wherein R¹ is Zₙ-(C═O)OR¹⁰, Zₙ-cycloalkyl, Zₙ-(C═O)OCH₂Ar, Zₙ-OR¹⁰,

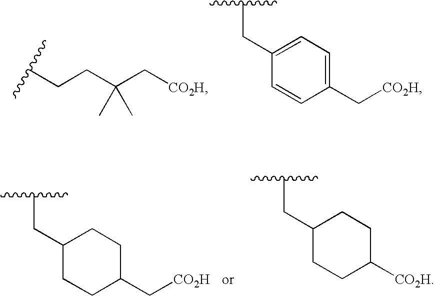

3. The compound of claim 1, wherein R² is alkyl.

4. The compound of claim 3, wherein R² is ethyl.

5. The compound of claim 1, wherein R⁴ is aryl optionally substituted with one or more alkyl groups.

6. The compound of claim 5, wherein R⁴ is 3,5-ditrifluoromethylphenyl.

7. The compound of claim 1, wherein R⁵ is 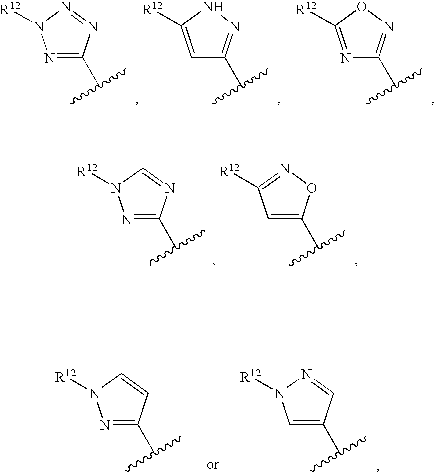
wherein R¹² is H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₘ-NR′R″, Zₙ-C(═O)NR′R″ or Zₘ-OR′.

8. The compound of claim 2, wherein R⁵ is

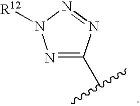

9. The compound of claim 8, wherein R¹² is H, alkyl, Zₙ-C(═O)OR′, Zₙ-CN, Zₘ-NR′R″, Zₙ-C(═O)NR′R″, Zₙ-cycloalkyl or Zₘ-OR′.

10. The compound of claim 1, wherein R⁷ is F, Cl, Br, I, CF₃, OCF₂H or alkyl.

11. A compound having the Formula: 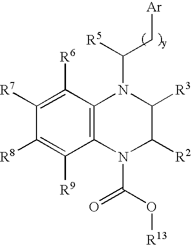
and metabolites, solvates, tautomers, resolved enantiomers, diastereomers, pharmaceutically acceptable salts and pharmaceutically acceptable prodrugs thereof, wherein:
R² is C₁-C₄ alkyl, C₂-C₄ allyl, C₂-C₄ alkenyl, C₂-C₄ alkynyl, C₁-C₄ heteroalkyl, C₂-C₄ heteroalkenyl, or C₂-C₄ heteroalkynyl, wherein said alkyl, alkenyl, alkynyl, heteroalkyl, heteroalkenyl and heteroalkynyl are optionally substituted with one or more groups independently selected from OR¹⁰ and SR¹⁰;
R³ is H, CF₃, Zₙ-NR¹¹R¹², Zₙ-NR¹⁰(C═O)R¹⁰R¹¹, Zₙ-SO₂R¹⁰, Zₙ-SOR¹¹, Zₙ-SR¹⁰, Zₙ-OR¹⁰, Zₙ-(C═O)R¹⁰, Zₙ-(C═O)OR¹⁰, Zₙ-O(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar are optionally substituted with one or more groups independently selected from OR¹⁰ and SR¹⁰; 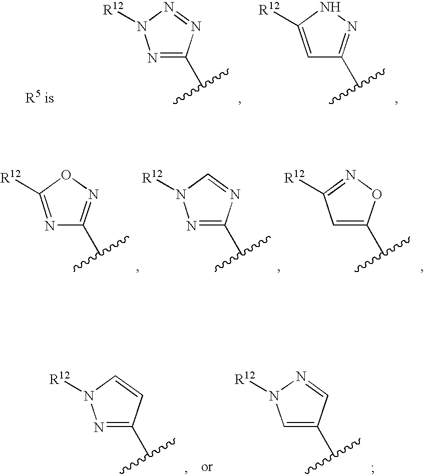
R⁶, R⁷, R⁸ and R⁹ are independently H, OH, F, Cl, Br, I, CF₃, OCF₃, OCF₂H, Zn-NR¹⁰R¹¹, Zₙ-(C═O)NR¹⁰R¹¹, Zₙ-SO₂R¹⁰, Zₙ-SOR¹⁰, Zₙ-SR¹⁰, Zₙ-OR¹⁰, Zₙ-(C═O)R¹⁰, Zₙ-(C═O)OR¹⁰, Zₙ-O—(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar,
or R⁶ and R⁷, R⁷ and R⁸, and/or R⁸ and R⁹ together with the atoms to which they are attached form a carbocyclic or heterocyclic ring, wherein said carbocyclic and heterocyclic rings are optionally substituted with one or more groups independently selected from alkyl and F;
R¹⁰ and R¹¹ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, cycloalkyl, heterocycloalkyl and Ar are optionally substituted with one or more groups independently selected from alkyl, OR′ and Ar,
or R¹⁰ and R¹¹ together with the atoms to which they are attached form a substituted or unsubstituted, saturated or partially unsaturated 5 or 6-membered heterocyclic ring;
R¹² is H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₘ-NR′R″, Zₙ-C(═O)NR′R″ or Zₘ-OR′;
R¹³ is C₁-C₄ alkyl, C₂-C₄ allyl, C₂-C₄ alkenyl, C₂-C₄ alkynyl, Zₙ-cycloalkyl, or Zₙ-Ar, wherein said alkyl, allyl, alkenyl, alkynyl, cycloalkyl and Ar are optionally substituted with one or more groups independently selected from OR′ and alkyl;
Ar is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, —OR′, SR′F, Cl, Br, I, CN and NO₂, wherein said alkyl is optionally substituted with one or more groups independently selected from C(═O)OR′, CN, NR′R″, C(═O)NR′R″, cycloalkyl, OR′, F and alkyl;
R′ and R″ are independently H or C₁-C₁₀ alkyl, wherein said alkyl is optionally substituted with one or more F;
Z is alkylene having from 1 to 4 carbons, or alkenylene or alkynylene each having from 2 to 4 carbons, wherein said alkylene, alkenylene and alkynylene are optionally substituted;
m is 1 or 2;
n is 0, 1, or 2;
and y is 0 or 1.

12. The compound of claim 11, wherein R⁵ is

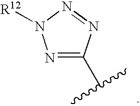

13. The compound of claim 12, wherein R¹² is H, alkyl, Zₙ-C(═O)OR′, Zₙ-CN, Zₘ-NR′R″, Zₙ-C(═O)NR′R″, Zₙ-cycloalkyl or Zₘ-OR′.

14. The compound of claim 11, wherein R² is alkyl.

15. The compound of claim 14, wherein R² is ethyl.

16. The compound of claim 11, wherein Ar is aryl optionally substituted with one or more alkyl groups.

17. The compound of claim 11, wherein y is 0.

18. The compound of claim 11, wherein R¹³ is alkyl or Zₙ-Ar.

19. A compound having the Formula: 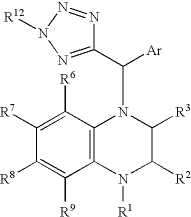
and metabolites, solvates, tautomers, resolved enantiomers, diastereomers, pharmaceutically acceptable salts and pharmaceutically acceptable prodrugs thereof, wherein:
R¹ is Zₙ-(C═O)OR¹⁰, Zₙ-(C═O)R¹⁰, Zₙ(C—O)Zₙ(C═O)OR¹⁰, Zₙ-NR¹⁰R¹¹, Zₙ-(C═O)NR¹⁰R¹¹, ZₙSOR¹⁰, Zₙ-SO₂R¹⁰, alkyl, allyl, alkenyl, alkynyl, heteroalkyl, heteroallyl, heteroalkenyl, heteroalkynyl, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, Zₙ-cycloalkyl, Zₙ-heterocycloalkyl and Zₙ-Ar are optionally substituted with one or more groups independently selected from F, Zₙ-COOR¹⁰, ZNOR¹⁰, Zₙ-NR¹⁰R¹¹, Zₙ-(C═O)NR¹⁰R¹¹, oxo and alkyl;
R² and R³ are independently H, OH, F, Cl, Br, I, CF₃, Zₙ-NR¹⁰R¹¹, Zₙ-NR⁰(C═O)R¹¹, Zₙ-SO₂R¹⁰, Zₙ-SOR¹⁰, Zₘ-SR¹⁰, Zₙ-OR¹¹, Zₙ-(C═O)R¹⁰, Zₙ-(C═O)OR¹⁰, Zₙ-O(C═O)R¹⁰, alkyl, allyl, alkenyl, alkynyl, heteroalkyl, heteroallyl, heteroalkenyl, heteroalkynyl, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar are optionally substituted with one or more groups independently selected from OR¹⁰ and SR¹⁰;
or R¹ and R² together with the atoms to which they are attached form a substituted or unsubstituted, saturated or partially unsaturated 5 or 6-membered heterocyclic ring;
R⁶, R⁷, R⁸ and R⁹ are independently H, OH, F, Cl, Br, I, CF₃, OCF₃, OCF₂H, Zn-NR¹⁰R¹¹, Zₙ-(C═O)NR¹⁰R¹¹, Zₙ-SO₂R¹¹, Zₙ-SOR¹⁰, Zₙ-SR¹¹, Zₙ-OR¹⁰, Zₙ-(C═O)R¹⁰, Zₙ-(C═O)OR¹⁰, Zₙ-O—(C═O)R¹⁰, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar,
or R⁶ and R⁷, R⁷ and R⁸, and/or R⁸ and R⁹ together with the atoms to which they are attached form a carbocyclic or heterocyclic ring, wherein said carbocyclic and heterocyclic rings are optionally substituted with one or more groups independently selected from alkyl and F;
R¹⁰ and R¹¹ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, cycloalkyl, heterocycloalkyl and Ar are optionally substituted with one or more groups independently selected from alkyl, OR′ and Ar,
or R¹⁰ and R¹¹ together with the atoms to which they are attached form a substituted or unsubstituted, saturated or partially unsaturated 5 or 6-membered heterocyclic ring;
R¹² is H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, Zₙ-C(═O)OR′, Zₙ-CN, Zₘ-NR′R″, Zₙ-C(═O)NR′R″ or Zₘ-OR′;
Z is alkylene having from 1 to 4 carbons, or alkenylene or alkynylene each having from 2 to 4 carbons, wherein said alkylene, alkenylene and alkynylene are optionally substituted;
Ar is aryl or heteroaryl, wherein said aryl and heteroaryl are optionally fused to a saturated, partially unsaturated or fully unsaturated carbocyclic or heterocyclic ring, wherein said aryl and heteroaryl are further optionally substituted with one or more groups independently selected from alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, Zₙ-Ar, CF₃, —OR′, SR′F, Cl, Br, I, CN and NO₂, wherein said alkyl is optionally substituted with one or more groups independently selected from C(═O)OR′, CN, —NR′R″, C(═O)NR′R″, cycloalkyl, OR′, F and alkyl;
R′ and R″ are independently H or C₁-C₁₀ alkyl, wherein said alkyl is optionally substituted with one or more F;
m is 1 or 2; and
n is 0, 1, or 2.

20. The compound of claim 19, wherein R¹² is H, alkyl, Zₙ-C(═O)OR′, Zₙ-CN, Zₘ-NR′R″, Zₙ-C(═O)NR′R″, Zₙ-cycloalkyl or Zₘ-OR′.

21. The compound of claim 19, wherein R² is alkyl.

22. The compound of claim 21, wherein R² is ethyl.

23. The compound of claim 19, wherein Ar is aryl optionally substituted with one or more alkyl groups.

24. The compound of claim 1 selected from the group consisting of:
4-[(3,5-bis-trifluoromethylphenyl)-(2-methyl-2H-tetrazol-5-yl)-methyl]-2-ethyl-3,4-dihydro-2H-quinoxaline-1-carboxylic acid ethyl ester;
4-[(3,5-bis-trifluoromethylphenyl)-(2-methyl-2H-tetrazol-5-yl)-methyl]-2-ethyl-6-trifluoromethyl-3,4-dihydro-2H-quinoxaline-1-carboxylic acid ethyl ester;
4-((3,5-bis(trifluoromethyl)phenyl)(2-methyl-2H-tetrazol-5-yl)methyl)-1-(cyclohexylmethyl)-2-ethyl-6-(trifluoromethyl)-1,2,3,4-tetrahydroquinoxaline;
4-((3,5-bis(trifluoromethyl)phenyl)(2-methyl-2H-tetrazol-5-yl)methyl)-1-(cyclopentylmethyl)-2-ethyl-6-(trifluoromethyl)-1,2,3,4-tetrahydroquinoxaline;
2-hydroxyethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate;
butyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate;
benzyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate;
isobutyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate;
and resolved enantiomers and diastereomers thereof.

25. The compound of claim 1 selected from the group consisting of:
2-(trans-4-((4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)methyl)cyclohexyl)acetic acid hydrochloride;
5-(4-((3,5-bis(trifluoromethyl)phenyl)(2-methyl-2H-tetrazol-5-yl)methyl)-2-ethyl-6-(trifluoromethyl)-3,4-dihydroquinoxalin-1(2H)-yl)pentan-1-ol;
and resolved enantiomers and diastereomers thereof.

26. The compound of claim 1 selected from the group consisting of:
ethyl 2-ethyl-4-((2-methyl-2H-tetrazol-5-yl)(3-(trifluoromethyl)phenyl)methyl)-6-(trifluoromethyl)-3,4-dihydroquinoxaline-1(2H)-carboxylate;
ethyl 2-ethyl-4-((2-methyl-2H-tetrazol-5-yl)(3-(1,1,2,2-tetrafluoroethoxy)phenyl)methyl)-6-(trifluoromethyl)-3,4-dihydroquinoxaline-1(2H)-carboxylate;
ethyl-4-((3,4-dichlorophenyl)(2-methyl-2H-tetrazol-5-yl)methyl)-2-ethyl-6-(trifluoromethyl)-3,4-dihydroquinoxaline-1(2H)-carboxylate;
and resolved enantiomers and diastereomers thereof.

27. The compound of claim 1 selected from the group consisting of:
ethyl 4-((3,5-bis(trifluoromethyl)phenyl)(2-(3-methoxy-3-oxopropyl)-2H-tetrazol-5-yl)methyl)-2-ethyl-6-(trifluoromethyl)-3,4-dihydroquinoxaline-1(2H)-carboxylate;
ethyl 4-((3,5-bis(trifluoromethyl)phenyl)(2-(2-cyanoethyl)-2H-tetrazol-5-yl)methyl)-2-ethyl-6-(trifluoromethyl)-3,4-dihydroquinoxaline-1(2H)-carboxylate;
ethyl 4-((3,5-bis(trifluoromethyl)phenyl)(2-(2-(dimethylamino)ethyl)-2H-tetrazol-5-yl)methyl)-2-ethyl-6-(trifluoromethyl)-3,4-dihydroquinoxaline-1(2H)-carboxylate;
ethyl 4-((3,5-bis(trifluoromethyl)phenyl)(2-(cyclopropylmethyl)-2H-tetrazol-5-yl)methyl)-2-ethyl-6-(trifluoromethyl)-3,4-dihydroquinoxaline-1(2H)-carboxylate;
ethyl 4-((3,5-bis(trifluoromethyl)phenyl)(2-(2-amino-2-oxoethyl)-2H-tetrazol-5-yl)methyl)-2-ethyl-6-(trifluoromethyl)-3,4-dihydroquinoxaline-1(2H)-carboxylate;
ethyl-4-((3,5-bis(trifluoromethyl)phenyl)(2-(2-hydroxyethyl)-2H-tetrazol-5-yl)methyl)-2-ethyl-6-(trifluoromethyl)-3,4-dihydroquinoxaline-1(2H)-carboxylate;
ethyl-4-((3,5-bis(trifluoromethyl)phenyl)(2-(2-hydroxyethyl)-2H-tetrazol-5-yl)methyl)-2-ethyl-6-(trifluoromethyl)-3,4-dihydroquinoxaline-1(2H)-carboxylate;
and resolved enantiomers and diastereomers thereof.

28. A pharmaceutical composition comprising a compound of claim 1 and a pharmaceutically acceptable carrier or diluent.

29. A kit for treating a CETP-mediated condition, wherein said kit comprises:
a) a first pharmaceutical composition comprising a compound of claim 1 or a pharmaceutically acceptable salt or prodrug thereof; and
b) optionally instructions for use.

30. The kit of claim 29, further comprising a second pharmaceutical composition comprising a second CETP inhibitor.

31. The kit of claim 30, further comprising instructions for the simultaneous, sequential or separate administration of said first and second pharmaceutical compositions to a patient in need thereof.

31. A compound according to any one of claims 1-27 for use as a medicament for the treatment of an abnormal cell growth condition in a human or animal.

32. The use of a compound according to any one of claims 1-27 in the manufacture of a medicament for the treatment of a CETP-mediated condition in a human or animal.

33. A method of treating a disorder or condition selected from cerebrovascular disease, coronary artery disease, ventricular dysfunction, cardiac arrhythmia, pulmonary vascular disease, reno-vascular disease, renal disease, splanchnic vascular disease, vascular hemostatic disease, diabetes, inflammatory disease, autoimmune disorders, immune function modulation, osteoporosis, pulmonary disease, anti-oxidant disease, sexual dysfunction, cognitive dysfunction, schistosomiasis and cancer in a mammal, comprising administering to said mammal a therapeutically effective amount of a compound of claim 1.

34. A method of decreasing small dense LDL, oxidized LDL, VLDL, apo(a) or Lp(a) levels and/or increasing the level of pre-beta HDL, HDL-1,-2 and 3 particles in plasma, comprising administering to a patient in need there of an effective amount of a compound of claim 1.
